SEQUENCE LISTING (294 sequences)

SEQ ID NO: 1 - DNA, 334 bp, Artificial Sequence
  Probe  1415802_at
ttctcttggtttttgatagttccatttataccactactagtttagagctgtatgtgggatgattcagtaccgactgaatggatgtgcttttgttttttttacattgtttttcagtatttgcaaaaccacgagggttagagtttggcctcagggaagccaataaagataaaatgggaggaagtttgctgagaacttgtatcatgctttacgattatttgacatagtcttacctccacaccttaactttcatgaccctttcactncacctganaatgtagaaaaatgggttcagtgtaaggataagaggaaagatggaccagattggaactacagt

SEQ ID NO: 2 - DNA, 183 bp, Artificial Sequence
  Probe  1415807_s_at
gctacagccgctccaagtctcggtcccgcanctcgnctcgcgctccagatcaacctcncaagtccagatctgcccgaagatccaagtccaagtccntcctcngtctccagatcccgctcgaggtcaaggtccaggtcgcgatcgagaagccctccgcccgtgcgaagcgagagtccaagtcta

SEQ ID NO: 3 - DNA, 313 bp, Artificial Sequence
  Probe  1415834_at
atttgcagcatgcttgaccttgatgtaccaattctgacggcattttcgtggatattattatcactaagactttgttatgatgaggtcttcagtctctttcatatatcttccttgtaactttttttttcctcttaatgtagttttgactctgccttacctttgtaaatatttggcttacagtgtctcaaggggtattttggaaagacaccaaaattgtgggttcactttttttttttttttnnnnnaaataacttcagctgtgctaaacagcatattacctctgtacaaaattcttcagggagtgtcacctcaa

SEQ ID NO: 4 - DNA, 448 bp, Artificial Sequence
  Probe  1419030_at
aaatctgagctgaactacttactataagccttaaaataaggagacatcccacatcccagccacctgacgtttgaatttggttgcctttttcgacctgtgtagtcacaggctatacaactcatgtttttacgcatgaaactctgtttttctccacaggatggagctgtaacgtagataggatgggttgttagtagaagctctacactgagagcagtatttgatgtggcttaagaatttacatcacatggcaattataaatggcatatacctcaaaattatgcccccttgaggtcacatctaatgtcttgagcagttaggactggttgagatgtcacataagaaaaagcaggacttacgtatgtaatggctaggaatgtcactttacactgttgtgtattttctctgtccctaagacttggtctagtgccaggcatacagttggtgtcta

SEQ ID NO: 5 - DNA, 196 bp, Artificial Sequence
  Probe  1419058_at
gcttgacctacaacaacggcaggttacagaacctcatttcaagttctatgagggccaagatcacttcgtatgcgattatacttgccctgcacataaataacttccaggttgacctgacagcgttgcagaaggacttgaagctgagtgagaaaaggatgattgagatagcgaaagccatgaggctgaagatctccaa

SEQ ID NO: 6 - DNA, 183 bp, Artificial Sequence
  Probe  1415910_s_at
gggtgctgctgtgtcttgaaagaccaaggattgaggggacctgtctcttagtgttaggggaagagggtggatgcgcctaaggaactctgctcctggtgttacagtngctntccctctccacccttgagtatttatgctgagtaagctccaggctccngctcagtgatgctgccacctgctgga

SEQ ID NO: 7 - DNA, 472 bp, Artificial Sequence
  Probe  1416022_at
ggcgcctgatggaaagccacggctttgaggagtacatgaaagagctaggagtaggactggctcttaggaagatggctgccatggccaagccagactgtatcattacgtgtgatggcaacaacatcacggtcaaaaccgagagcacagtgaagacgacngtgttctcttgtaacctgggagagaagtttgatgaaacgacagctgatggcagaaaaactgagacggtctgcaccttccaagacggtgccctggtccagcaccagcaatgggacgggaaggagagcacgataacaagaaaactgaaggatgggaagatgatcgtggagtgtgtcatgaacaatgccacctgcactcgggtctatgagaaggtgcaatgagggcttcctncgtcatcctggacaggagttagctgccagagtgaatatgctcaattcagtgagcggtcaaatgcaacaaacagcttcacttcttt

SEQ ID NO: 8 - DNA, 174 bp, Artificial Sequence
  Probe  1416070_a_at
agccatgatggcaagcttaagaagagaggcggtggtggcggattcggctaccagaaaacaaagaaagtggagaagtccaagatctttaaacacatcagtaagaagccagcagaccgcaggcagttctcacactgaggcaatctnccttcagcctggactgactttgcccagctt

SEQ ID NO: 9 - DNA, 285 bp, Artificial Sequence
  Probe  1416071_at
gcgccctgaagagttgggtttccttcgttacttgaaacagtccaaggttccattgaatcagtttgacttctcctggtctaaagtttctgatatccagtctcagctggaaaaactgatcgaaaagaactactttctccacaagtctgcccaggaagcatacaaatcctacataagagcatatgactctcactctctcaagcagatcttcaacgtgaacaacttaaatctgcctcaggttgctctgtcttttggttttaaggtccctccttttgttgatctgaatgt

SEQ ID NO: 10 - DNA, 269 bp, Artificial Sequence
  Probe  1416122_at
tcacgacttcattgagcacatccttcgcaagctgccccagcaaaaggagaagctgtccctgatccgcaagcatgcgcagaccttcatcgctctgtgcgctaccgacttcaagtttgccatgtacccgccntcgatgattgcaactggaagcgtgggagcagccatctgtgggcttcagcaggatgatgaagtgaacacactcacgtgtgatgccctgacngagctgctggccaagatcacccacactgatgtggattgtctcaaagcct

SEQ ID NO: 11 - DNA, 277 bp, Artificial Sequence
  Probe  1416126_at
atcgacaccgtctctgtgccgtatgtcttccggtactttgtagctgagctggcagccnatgaacatcaaagtgaaactggatgtcatctgacttgatccccgccatctgagctaggagataagaacaggaggtgtcctgtataacacaagtgagggtattatgggctcactctgggtctatacaagaatttcagtactgaagccaagaacaggcaggtgaaaattcaagacttctgagtctctgtaacctanctgaagttggttttcatatcttttt

SEQ ID NO: 12 - DNA, 360 bp, Artificial Sequence
  Probe  1419518_at
agagataactagctcctgtttcgagcccaacagccagatggtgaagtgtgacccacgtcatggcaaatacatggcctgctgcatgctctaccgcggtgatgtggtacccaaggacgtgaatgtcgccattgctgccatcaagaccaagagaactattcagtttgttgactggtgtcccacaggtttcaaggtgggcatcaactaccagccacctactgtcgtgccaggaggggacctggccaaagtccagcgagccgtgtgcatgctgagcaacaccacagcaatcgcagaggcctgggcccgccttgaccataagtttgacctcatntacgccaagcgggcctttgtacattggtatgt

SEQ ID NO: 13 - DNA, 340 bp, Artificial Sequence
  Probe  1419522_at
attcctgtacctactatgagtgtcattatcctccttgtacggtgattgaaaagcagcttcgagagttcaacatctgtgggcgttgccaggtggctcgatactgtggctctcagtgccagcagaaggactggcctgcccacaagaagcactgtcgggagaggaagcgtcccttccagcatgagcttgagccagagcgatgacgggctgaagagctgccactgctgcaggttcaaagtttccagctcctcatgagcacagcggaagacagactgtcagttgaacctggaggtgccaaagaagccancaatggatccagagcagctgccataagacctcacag

SEQ ID NO: 14 - DNA, 389 bp, Artificial Sequence
  Probe  1419557_a_at
tccattggaggaccccgggcaaacactgtcctggagcgggtggaaggcgctcagcagcggtggaagctgcaggtgcaggagcagcggaagacggtcttcgaccgacacaagatgctcagttagatggttgccatgattgcatcagagacctgggccatggctaccangcnttcnggggctcactgcagtcttccctgggtcttncccttcaaatgcccatggcgtttatccttctccctctctagaaatgtactcgactgttataacgagggagtgtgattgggtcnnngtaggtctcctcggctcctggtccaccagtgcatggcagtgcccatgcatgccggcatattcagcagctgtcaccttactcccatcccaggaggccgtaa

SEQ ID NO: 15 - DNA, 543 bp, Artificial Sequence
  Probe  1416345_at
tcaaggtccctttactgctttataaaataattttgataggtggactgctttctagaatttgatagtgggaccacatagggtaaatcttgatttgtatgggttcatttctgatttttttagggagatctttgtgtcagtcaaattggcagtggggaaagatcctggcactgtttaactgtgagctttctttcatgtctgacctggagctgaaaaaaagtcctatggagtgttcctgcaatccaacttcttaggttgtctctcccctatactgtgacttacttaactcgcaaattgaaaatatggtctcaaggtagaagaggaagcctgagaatccaaagggctttagtcataaaagaatctagctcatgagtactattcttattaccagacagccacgagaagaagttaacttactatgtatatccagatgcaaaacattcacagatgtgcctaaagaaaccgcgacttaggccagttcagaactaacaactgtttcaggtagaggtgcatgcctgatggtagacatcgattccattaactgca

SEQ ID NO: 16 - DNA, 442 bp, Artificial Sequence
  Probe  1416346_at
gtcgtgccgggatggagtcctccacgtcctcttcgggctcggctctgggcgccgtggaccctcagttgcagcatttcatcgaggtggagacgcagaagcagcgcttccagcagctggtgcaccagatgacggaactttgctgggagaagtgcatggataagcctgggcctaagttggacagtcgggctgaggcctgttttgtgaactgcgttgaacgcttcattgatacaagccagttcatcttaaatcgactggaacagacccagaagtccaaacccgtcttctcagaaagcctttctgactgatctcagcattacctctttggaaaagtagttcaagaattgaagagctgctgatgaggtggttgaagaaatggctatgggggggattaaactcccaattttctttatcgtncttgggacatcttgtcacctgagaatga

SEQ ID NO: 17 - DNA, 201 bp, Artificial Sequence
  Probe  1416376_at
agtggtgggagacgtgccaggccttcacctttcagcaggggtgtgaccaagttacagcaaaatggtgccaaagcgctaccacagcactgctaaaaaggccaggtgggaagccggttagctcctgacctggaatgcacacacctgaccaagtcctcacctcgaccttgatagtcaacaaatgagcagtattttgtctcttta

SEQ ID NO: 18 - DNA, 298 bp, Artificial Sequence
  Probe  1416442_at
gggacacctagatcttgaatctcagggtcgaactctctaggggccaggctgccctttcaaggccgtttcactaccattcgcgtttcggcccctacaagtgggcacgcttgtgcaagcggtcagagttgcgtcatgggacagacgcgggtgcttcctgttgccttgcgtgggtgtggggcctgggaggaggccagggtgtggacccgccctagggactgggaagtgacttgagtcacctncgcccccacaggctgctgtgggtgagcctgaactgaaccaatcaaatctgcgatcagag

SEQ ID NO: 19 - DNA, 374 bp, Artificial Sequence
  Probe  1416445_at
agccttatcctgttggtggaatactgtaccatatgctcctccaaaacccatgaacacggccaaaacccataaaaacaacaaaagcaaactaagtcaagaaccgtagtatctgtaggcacttagttaagtatagcaactcaaaccagacctgcatccatccgagacaaacgttcctccttcacccttgtttttacttgaaattcgctagaagaaatggcgagcagacatctgttccatgcactagttaatgctactggctcagcatatacaagttagcttgctttaggcaagggacctttcttgtgatggacgtgcactacgaagtgagatggantcctgctccgtgcaggaggcccttcattattgctgcagaa

SEQ ID NO: 20 - DNA, 453 bp, Artificial Sequence
  Probe  1416448_at
gggccctgcacaagctacaggagtactttagtgtggctgctggggctggtgaccactagcgccatgggtaggggctagtgcagaactccctaggaggcaggcactcctgtgaggctttctctgggttcccctctgccaggagatccaggcagcatcaggagccagttcttggaagagcagttggctctgcataggggaactctgagccagttgatactaaactcttgtcccttggattcaagagtttcctttgaccaggcatggtagcgcacacttttaatcccagaatttgggaggcagaggcaaacagatctttgtgagctcagggccagcctggtttacatagtgaattcctggatagccagagctacatagtgagaccttgtctcaaaaacaaaacaaagaaaccaaaacaacaacaacaacaaaaagttgttcttgtagcctctggct

SEQ ID NO: 21 - DNA, 453 bp, Artificial Sequence; identical to SEQ ID NO: 20
  Probe  1416538_at

SEQ ID NO: 22 - DNA, 562 bp, Artificial Sequence
  Probe  1416539_at
gcagcacatctaccggactggaagaggagagaacacaggagaatggtgctccaaaccggttgtggttttgtatcagtttactagagcatgctccttttattatataaaaagcaggggctggtgatctgatattcctaactagggtcagaatcaagtatactgaattttcagcactgtgtatgtcatgaatcagcagctgccacggactcaacttgttaatcctaaaccctcgtgatgttatagttatagcctcgggaggagagtaggtcagaatgactctgcatagaattagtttccttataaaagggatccaagaactccctcccattatgccgtcatagcaagacagcaatctaagaaccagggggcaagccatcaccagacatcttggactttgacttcctgggcccccaaactcttgaaagataaattcactgtctataagcnanctggcttattgtattttgttatagcagccgaacagactaagtgcctttataaagacttaagttcatctcagggagagctaaacaaaaatgatcatatcccgactacaagaaga

SEQ ID NO: 23 - DNA, 562 bp, Artificial Sequence; identical to SEQ ID NO: 22
  Probe  1416559_at

SEQ ID NO: 24 - DNA, 505 bp, Artificial Sequence
  Probe  1416563_at
gctggctgtggttaagggccatcaaccaccttgttttccagccactcgagtcactgtcattggagtgaattgctgttgtctccctggggtgccaggactcccggtgctgggctgctaactgggtgttgctgacatctcagtgctgctgagttacaggggactaggtgccagctgtggttgccctcgccatccagagggttgtttggttctttggtacatcaaactctgtctgtataaagtatcatataaaaaccccnggntctctggctacactttctattcctggggctgagaaaactccttgncatcaaggggtcaagaaacacggaattttagggtaaccggttctaaanccctntcttgtgtgtgcaccgttggcttgaaggtattttttttcttccagaatggtgttattgcatttgaaatgcaatttgaagttattttcttaatgtgatatgncaatgggcatgaccatgtncctacagtatggtggttagcttgatgc

SEQ ID NO: 25 - DNA, 189 bp, Artificial Sequence
  Probe  1416605_at
gcctgacgcgcaagctctacaagtgcatcaagaaggctgtgaagcagaagcagattcgtcgcggggtgaaggaggttcagaaatttgtcaacaagggcgagaaagggatcatggttttggcaggagatacattgccgattgaggtgtactgccatcttccagttctgtgcgaggaccagaacttgccct

SEQ ID NO: 26 - DNA, 392 bp, Artificial Sequence
  Probe  1416750_at
gtttgaacacacatcacaagcttgactcactgactcaggctttcctagagcctcctgctggcattcagggctctgggatcaagggttgaggtagatgttacacactacctcactctgatgcctcattcctgtagtaatctctggtgaagggaaagaggagctgcccttcggaggtccccttcacctgcagnctatgatgcccttcccttcatccctttcctcaccatatgccttatctccattccactcccctgctatgcaaatgcccnnnnnnccntnncnntnncctncccnnccccccccccccggcaaccagttcagctgggaaagcagaggcatgtagaggatgctaaagggaagggaggtcctccactacggagacccttgttggg

SEQ ID NO: 27 - DNA, 430 bp, Artificial Sequence
  Probe  1416795_at
cgggcctgttccagagttctcaggggccacggtggagagagttagtgaggacatgtgcatgaaggttcctgatgatccggagcacttagctgccaggaggcagtggagggacgactgcctcatgaagctctccatactgaagtaccagatgcagcccaagtgaatttcctgtaatgctgctgcccctccttcctgggcagctctactggggagatgagaagctcttgatccaatccctgtgcagcatcaagaagcccaggaagctggcccaggcctgctcctctgtgtttccccatgggcagcgatagaggccnaagtatcttgtggncantcctnccnnnnaccctncnattggganatcgaagcactgcttaggttctgaggtgtgggttccctnccacccaggcaaacaggctgaagattctcacct

SEQ ID NO: 28 - DNA, 501 bp, Artificial Sequence
  Probe  1416864_at
cttctctccctcaagctgcttttggtcatgttgttttgttttatcacagcaatagaaacccaaagataagtctgtgcttttggaggaaaggaggcagggcagggtggctcctgggatgctggctaggctgaggttgtgagtggagatagggctacttttaactaggtgacaggaatggccagacataggtgcagagccaccctcccgggtcagagcttgggtgcaggggtgggctggtagggctggggtcatttgtaattactaaaggctgaacagaattaagaagtgcttgttgtgtgtggtggcacacaccgttantcccattactcaggaggcataggcaggtggatntnttcagtttgagnccagccnagggctacgtagagacctgtgtaagaaaacaacaataacaaaacacagatctagatagaagtgttggcacattgctgtaatcccagcactggagtaagagtcattaagcttccaggtaacctcattgta

SEQ ID NO: 29 - DNA, 400 bp, Artificial Sequence
  Probe  1420056_s_at
gacgcagttgacctccaggagtccacaggcattgcctctgacagctccagcgactcttctagctcctctagttccagctcgtcagactcggactcagantgtgaatctgggtcagaagntgatgggacgacacaccgcaggaagaagaggagaacctgcagcatggtgggaaatggggacactacctcccaggatgactgtgtgagcaaagagcgcagctcctccaggtgaccacatgtggccgctgtctgtgnccaggatatgctcgcagccaggcttggggggctgacacccagagaacaaggacactcagatgggagttggtagtacgtggtacagtggcttttctctgtggtacatgtcttaggggcaaaggcgtgtggatggtgccaatgaaacc

SEQ ID NO: 30 - DNA, 522 bp, Artificial Sequence
  Probe  1416890_at
gtcctagaggccacctatggagagtacccactgacagctatgacccttactcctgagggcaattccgtcatcgtagggaacactcatgggcagctggcagaaattgattttcggcaagggcgtctactgggctgtctgaaggggcttgcaggtagtgttcgtgggctgcagtgtcacccttcaaagcccctattagcctcatgtggcttggacagagtcttgaggatacacaggatccgcaatccacgtgggctagagcataaggtttatntcaagtctcaactgaactgccttctcctgtcaggccgggataactgggaggatgagccccaagaacctcaagagcccaaccaagtaccctcagaagacacagagacagatgaactttgggcctntttggaggcagctgccaagcggaagntcccagatttggaccagacacaaggtgntntccaaagaaggaagaaaaagaagcggcctggatccaccantccttgaagccccttgctcatgttgttaata

SEQ ID NO: 31 - DNA, 334 bp, Artificial Sequence
  Probe  1416962_at
ggttttgctgcccagactctgaaaagttgggactctgtgaagccctgcttaccacgagcctctttctgttcctaacagtagtttacaaggacacagatggtttgtattgtcatcggatccagttgtcatggacccatgcctggggagatctggacaaaggcttttcaaggctctgagtgatctaagccaaatatttgactctaaacaggtgtccatgggcaaaaacactgacccacttgaaaaaaaccaactgtcctcccaaagcacaaggcatcccagtatcccttgggtgtcacctgtcccttccccatcagcctttctgccatagaggact

SEQ ID NO: 32 - DNA, 133 bp, Artificial Sequence
  Probe  1416998_at
taacaaagcttgtgtatttatcaaagcggggagggcgggggaaaattatatctacctgtgatttgcaagtattgtaaatggatgcaggtacctggtgttgcttttaacttttactgtcggtagaggttgcatg

SEQ ID NO: 33 - DNA, 535 bp, Artificial Sequence
  Probe  1417035_at
gtggaagcccctgcaggaggttctacagctgcctgctgcccctgcgtgcctgcccaccccttcaggcggcgttggctgttgacgctgccttccgggaagacaaccatgcccgactgtttcgccntgcttcgcaccttaccctacctacagagctgcgcagtgcaggaacacattgggtatgcccgccgcaaagctctagcccgcctgtcccgtgccctgagcactcctaagggacagaccttgcctttggacttcatagaacactttctggccctggatggactccaggaagcacgggacctgtgccaggcccatggactgaccttggataaagacagagttgtattcctgagggntcaatantntgaggaaggactcccaccccctggtncctaccacatattagtggggaataagttgcagggacacaccctggaagatgtgntcatggctgaggaaggtgacattcacagacctggntntgcagcttgagagggtntcntntcccacaaggcctgggtcagagcacctggct

SEQ ID NO: 34 - DNA, 547 bp, Artificial Sequence
  Probe  1417064_at
caagttcatgcctggcaactgtactacagcaaaaaactcttagactcttggttcaccagcacacaggagaagaaacggaaatgaagcctgcttgataaactgctctgaggggtaaaacctaggtctcccattgagcagcgtgaagggagctgtccagactctccatcgattgtggcatctgtgatgttggcacctagtgcacactaggctcagattttggcctagagttctgttaccatctgctgaggtggcaaagctgttgtatttaatttatgcttgactagctgcagctgggtgaccaatggctgtgacacactttacctggttgaacttgaggccctttagagtttgggttttccagactgagccttatccttgccttctnttggccngtactccctccttccattcgtcacctcttcaccaccagtactagaagaaatatggaataaatcagtacatgaaagcagacttcagtctgtgggtcatgggcaagcaacatttgggaagttgctcccctatgggctgttctgtttactgcaaacca

SEQ ID NO: 35 - DNA, 455 bp, Artificial Sequence
  Probe  1417066_at
ggagccacggcctctatgaataggaagagagcgttgcgtagaccctcctattgcttctgtactgtccagacaaaaaggcaggcttcttttgccatctcctccctgctgtggatgtacaaccaggaacaggagcaatgcagtactaaactcaacacagccacctttggggtctagttggccatgggaatgaattgtgtgtttctgggtgagcttatgtccctgccctttgccaccccagcctgtggcctggagctagaagctcaggacagcagggtagccaccctagctcagtctcagcatggacaacagacccttttaaaagcagggctgacattggatgagcagcagggctccagcatgccccaggagagggagctatcaccccaccccaggggagcgacacaggctctttgtttgtatgtnttgtaattacacattcatacctttgcgttcaa

SEQ ID NO: 36 - DNA, 495 bp, Artificial Sequence
  Probe  1417132_at
agtggtgaatcacatcagttaccccctaatctaatcccatggaagtgaggctctcggaaacacctctcgtctgaggggccaggnctgctttgattctgcacagcctgttttggtgcctgcctgttgatatccttggacaccagcctctctgctatctttgctccattggggtctagccaggtttttcttaggaagagtctcccctcttccctctgctttctattcngggggcggggagggnaatcagtgatattgaagatggctanttgctttnttatgggtttgagttgcatttggctattaaacaaatcttgtttgaaaatatgtggagancaagggaatgagcanccccttccctggcgtgttgaagtatntcccagttgntccctgntctggtttgtagagattctgttagctgaatgctttccaggagaatggcttgcagattgtaccagcatagctagcattgttaaccaacagttgcaggctctgcaa

SEQ ID NO: 37 - DNA, 421 bp, Artificial Sequence
  Probe  1417212_at
tattcaatcgagtaaacggccggcgacccctcaccacctccccatccctcgaggggacccaagagacctacacagtggcccacgaggagaacgtccgatttgtgtccgaagcctggcagcaggtagagcgacaactggatggcggccctgccgatgagagtggcccacggcctgtgcagtatgtggagagcactcctgatccccggctacagaactttgtacctattgacctcgatgagtggtgggcacagcagtttctggctaaaatcaccaactgttcctagcagctgtccgggaaggaatgttgccatagtggaccctttngccagaagaggaccatggtgcnccgatccaccttgtggcctggctgggtaccggccacacctgaagtgccaacatttggacttttgcacctgttgtt

SEQ ID NO: 38 - DNA, 536 bp, Artificial Sequence
  Probe  1417218_at
aggtaatggcacagcccctgacaatgtggagatggccctgctcactgcttaagagcctgcttctatgctctgtaagcggaactagtcacagactaantcctcatggcgtgctcatttcagcatcaggtgacttttctgctanctacagtgtcttgggcatagacataaaacaaccagtagtttntaaagatcctgtggaccacaaggttcacagaaattgaggtggggtgagggtaggggagggggtagggactggcctaccgaantcatttctncccgtgnccactancacactctgctcacagctatcagaaaggtactttcctagagctgntctttagtatgacaggnccgatntnaggtggncctcagagttgagaatcatcagctggtgaancctgttgacaacccactgggaanccttcattgtatagtacactttgtgcagttaatttgtgtctaattgcataggagcaccccagctcccagtgtactgctaggaagctgcccntgattgtatatctctggccagattt

SEQ ID NO: 39 - DNA, 456 bp, Artificial Sequence
  Probe  1417233_at
gtgtcctgcataaacctgtccttaaacattagtccactcctggatgtactactctgtgattgccctggattttgccactttgaaataatgtgttgagacttctcagcaaccaaaagttgttactcaggaaagtttatcgtgagaaagctgatctattctgatccattttggtatcccttttggtagattttacacctcttttggaaattaagtaaatagatgccaaaactcctttaagagtgggtccattcctactggaagaggctggtggtcaggtgcttgctggagtacatggggctgctgcctcgttcccgacagctaattgtctgtttgctgtccaggctccagtgcctacagctgcagggtggcattggatcctgtgactcccggtccttcctaagtgaaaggatgggctgggtttgcagtttattgttttgctgttctaagagccctggt

SEQ ID NO: 40 - DNA, 501 bp, Artificial Sequence
  Probe  1420463_at
tggagaatacagtcgccaggcagtggaagatgtgttaatgaaagagaacaaggatggtacttttttggtccgagactgctctacaaaatccaaggcagaaccatatgttttggtggtgttttatgggaacaaggtctacaatgtgaaaatccgtttcctcgagagcaatcaacagtttgccctgggcacaggactacgaggaaatgagatgtttgattctgtggaagacatcattgaacactacacatattttcccattctgctaatagatgggaaagacaaggctgcacgcaggaaacagtgctacctcacccagccactgcctctcgccaggctccttctcactcagtactccagccaggcacttcatgagtaagaagcccagccagatatccccgcatcagtggcctgggccttgtctcattcctggctcaatggattcagttcttcttccatctgcatttatctgcaaagtattattttctgtgtcttcaagggatg

SEQ ID NO: 41 - DNA, 451 bp, Artificial Sequence
  Probe  1417675_a_at
aactgaagcagctttttgtgcctcccagtggctgcctcaggagcctatgcaataatggacaggtgattctccctcatttcattcttgggctgccatagcccctgctgtaatttatttctttagccatccttcccccgatactaagaggaaaaattattcctcatactgctttcaagctatcctaagccagtgtcactgtggaaaggagcgaacggatgctgcgtccacgagaacttgacagctccatcctcccctcacccgcacagcacggtagtgttaggcttggttcccttgggcctgggggcagttttacttggccagaaagcagtgtttgaatacttgaaatcatgtgttgtgttctgtttaaatgttgtttgtagacattgtgctgtacacctgtctccatgtctgtttgcagatgacactgtgaagtgtgccacccttatatggt

SEQ ID NO: 42 - DNA, 533 bp, Artificial Sequence
  Probe  1417718_at
agaagattgtgtcctgccgcatttgcaagggcgaccattggaccacccgttgcccctacaaggacacactaggacccatgcagaaggagctggctgagcagctcggcctgtctactggggagaaggagaaactgccaggagagctggagcccgtgcaggccgcccagagcaagacagggaagtatgtgccccccagcctccgggacggggccagccngccgtggggagtccatgcagccaaaccgcagagctgacgacaatgccaccatccgtgtcactaacctgtccgaggacactcgtgagactgacttgcaggaactcttccggcctttcggctccatctcccgaatctacttggccaaggacaagactactgggcagtccaagggttttgcttttatcagctttcaccgccgggaggatgctgcacgtgccattgcaggggtgtctggttttggctacgaccatcttatcctcaatgtcgagtgggccaagccatcaacgaattaataagacatctgctacctgctgct

SEQ ID NO: 43 - DNA, 460 bp, Artificial Sequence
  Probe  1417726_at
aggatcgcatctcccgtctgatgggggactacctgctccgtggttaccgcatgctgggcgacacgtgcgccgactgcgggacgatcctcctgcaagataaacagcggaaaatctactgtgtggcttgtcaggagctcgactcagacgtggataaagataacccggcactgaatgcacaggcggcgctctctcaagctcgggaacaccagctggcctcatctacagagcctgcctcgagctctcggcccccatcccagccaccagtaccccgtccagagcactgtgaaggcgcagcagccggggctcaaggcagcccaggcgcctcctcttccggctgcacctccaaatacggacgcggtggcttccacacagacggccctcctgcagaagctaacctgggcgtcagtcgagctgggctctagcacctccttggagactagtatccagctctgtgggctta

SEQ ID NO: 44 - DNA, 528 bp, Artificial Sequence
  Probe  1417775_at
gggaagatcaccgtgagccacatttgctgttgggaatcccaggtgccaccgttgctgtgcgtatgctagtggctaaggaaacattcccaggggttcctgaagatccagaaagggcatgaaacctgctccatgtgcctgtccctccctgctgcagctaccaaaggagagtcctcaaagaggctccactcagggctgctttcctgggcctgagtcaatggcacaaggcccccgccacactgccattgctgtatggctgcatacaccagaccagaacactcccacagccaagacatctctctgggaccgcagcagctccctaccccacagcctaaagtttcctactggggctattcagcaagggccacgtagcttgaccttcctgcctctacactcagctggatgtcagagcccctagtcacccccaggccctgtactgttggggtctttgagccatgcagtcaggttccctggctggacagaaaagacagaggctttatgaagccagaccgtttctatttttgtccactg

SEQ ID NO: 45 - DNA, 371 bp, Artificial Sequence
  Probe  1417873_at
agcgaatctcacctatctccatcttcccaggctaggattacctatatgcatggtaggttctagaattgaattcaggccctcgtgttggcaaatcaaacatttcactaactgagcagtgtccctaacccattgataaatgtgatccctaaaggtattttcaccttttgaatgttaatcatattttgacaacttttggttttaaacttccatgttttcagggatattttgtttaaaatgtcaattattttttaaatggggattttgttcatacaattttctatgatcaaaatattttactaaatagacaacaaactgtcaaagtaatacataaccttgtaagttttggtcccagtacattctcccatctattt

SEQ ID NO: 46 - DNA, 477 bp, Artificial Sequence
  Probe  1421089_a_at
gatgtagagaactctccccaacacaaggccaagaggcataaaaagaaaaagcgtgtggaagcagagaggggccctgtggccaagaagagagaccgagcagagctgcagcctggaggccccagtgaggacgagtgttccgatgcctctgtcgaagctgcagaagattgtgtacaaacaccagacatccaggatgatgtcccaaagcccaagaagaggaaagcaaagaaaaagctgcaaaggccagaaggagtagaaatagacgccacactagacagagcaccggtgaaaaagaagaagaagaaagtttccagataacttgtggcctccaaccactcaactgtcagcacactgcagggcctgaagtgagagcacggcgaagcccaggtggctcagcatcttttaacatgcccacaggttgctgcttctacgcaccacatttccacatttcccccaggtcaccttcctaggagaactgat

SEQ ID NO: 47 - DNA, 528 bp, Artificial Sequence
  Probe  1417896_at
ggcactagtgactcctggtaccaggaagtcaaggctgtaatacagcaacagcaggcgcgaccgatctggaccgcggaggaccagctgaacagctcatccgaggaccttgacctgccgggccatggtctggccgccagctctggagacctcagctgtgacagtcggaccaacagcgactatgaggacaccgacggcgagggcgcctatacggacagagagggcggaccccaggatgtcgatgaggaggtcgcacccacagccctggcccggtcctcagagcccgtatgggtagatgatcaccaggggctgatgggtcatgggacaactatcacagacaaatgggagacacaggctgacagccactacacccaggaccagcggcgccaggacagcatgcggacctacaagcacgaggccctgaggaagaagttcacaagagcccgggatgtggaatcctctgatgacgagggctatgactggggcccagccaccgacctgtgacctatgcagtcaccagcgtgccacaag

SEQ ID NO: 48 - DNA, 536 bp, Artificial Sequence
  Probe  1421260_a_at
tgaggcacttctgcaaatctctcttccccgtggtggactacgcctactgtagcattcctacctatcccagcggccagatcggcttcatgctgtgtagcaaaaacccgagcaccaacttccgggagccagtgcagcagttgacacaggcccaggtggagcagatgcagctgaaatactataactcggacatgcaccgtgccgccttcgtactgcctgagttcacccggaaggccctcaatgacataagctgaatccaggtgccactgtgacaccacccgagacctcaatcggattggaccaaggatcttccaagttgtctggggaccaccagtcctggaccagactcccagatgactcttgcccaccaaccaagtgttacaggccccatgatgctgcctggcctggcctggcctggcctggcctggcctgccctgctgggtggactcagtctctgtctgtctatctctgtggcgttcagccccacgcctataccagctctgtacagcaccgcctatgccctgtgacccaacaaaata

SEQ ID NO: 49 - DNA, 563 bp, Artificial Sequence
  Probe  1418079_at
gcaaccagcagcttgtggacattattgagaaagtaaaacccgagattcggctgctgatcgagaaatgtaacacggtcaaaatgtgggttcagctgttgattcccagaatagaagatgggaacaacttcggggtatcaattcaggaggaaacagttgctgaactaagaactgtggagagtgaagcccgcatcttacctggaccagatttctagatattatattacaagagccaaattggtttctaaaatagctaaatatccccatgtggaggactatcgccgcactgtcacagagattgatgagaaagaatacatcagcctccggctcatcatctcagagctgaggaatcagtatgtcactctccatgacatgatcctgaaaaacattgagaagatcaaacggccccggagcagcaatgcagagacactgtactgaggccagggccagggccaggggactttgtgagtctggctcaagaccgacattgccttggtttgttacatgactatcgtgatgggaaaactggctggaaatagtaatcacacctctctgtttttagttag

SEQ ID NO: 50 - DNA, 531 bp, Artificial Sequence
  Probe  1418225_at
gtcctgcttctcctgagatgatggtagaagatgtcagtggactgagatgacacanccttntccttngtggacttagnccacacatgctaaataaagacanccancactgttcaaatgagatcacgggagaagtacaagggatgtgtgcatatgtggagcatgtgtccaatctagaaagcatatttaaaatcgttggtatcggtaccaggagaagccaggctaagaaataaantcctgatttacattttattgttttcagtacagtagaaaataaaaacatttcatcattcttgtacattgacattttctattttcttaaatagctcacagggataggtagcgcacacctttaatcccagcactcaggagtcagaggcagacggattgcttctctcctttaagacacaagacctgagctcacatttgtaataccaacatcaagaaggcagaggcagaggaaaacatgagttcaagacctagactagttagtgagttctagaccantctaagctgcggtgatgtcctgtttca

SEQ ID NO: 51 - DNA, 550 bp, Artificial Sequence
  Probe  1418226_at
agaaacctcctatgagaactcccttctggttaagcagtctggatctctgccacttagttctctgattcatgtcttacgaagccttacccccaatgcaagggggattttcaggctacttatgaagttccagctggataaccaggacagcccttcctacattggactttcttttcnagatttttaccagcagtgtagggaagcattccttgttaacagtgatctcacactccgagcccagttaactgaatttagggaccacaaacttataagaacaaagaagggaactgatggtgtagaatatttattaattcctgttgacagcggaatattggctgatttcttggagaaggaagaggaggaggcataggctgtctcttatccttgaactttctgtggaagcctcattgtggcccagctgctgctcctcagtaagaatgctggtttacatccaacattaggtcattctctccagcatacaagatgttctatggaagaggcatagcatcaatttgaactttgattagcctggctggtgtactgtctctaatac

SEQ ID NO: 52 - DNA, 509 bp, Artificial Sequence
  Probe  1418305_s_at
ggcatcttcctttaaaaagctacagaagttctatatagacccatacaagctgctgccgctgcagaggtttctgcctcgtcctcctggtgagaaaggacctcccagaggtggcggcggnnntggcggcaggggaggtcgaggaggaggaagaggaggcggtggccgaggtggtggaagaggtggtggttttagaggaggcagaggaggaggtggnngggcttcagaggaggnngaggaggnggcggattccgaggaaggggacattagctgtccagttggcagacctgaccacttgtgttcccgctgcgtggcctgcttctgctatggttgacgtctgtgtctggagcagtcttgaggaggccattccgtcagtggagctgantcagcagcatcgcgtagaaacaaggggagcagggcagttagntctgctcagagaggagaaccccgctttgtacagaacctggcactctgtgggtgcttagagaagacaggacttttcatttgaagca

SEQ ID NO: 53 - DNA, 415 bp, Artificial Sequence
  Probe  1418411_at
gctgcgcgggcttgagagaaatacactgcttctgcgtggtgagaccctcggtactggacgccttccgagcgcactgtccgcgcctgcgcagctatacgctcaagctaaagcgtgagccgcatccctggcggcccacactggtncggtgattgggcaatctagcccccccagggccgcgcgaaccccaagcctcgggaaggcctaagcaagttgcgcaggcacgccccagttgctagtctctttaggggctccgttgactaaggcgctttnggggaactgggggtctatgaaagcatccagaccaatnctcaggcaccataaattcagcgagcccntctcaacctctgtgtctctgtctattctggnccctccntcccctctgtacactcncactagctctagctcaggtccagag

SEQ ID NO: 54 - DNA, 262 bp, Artificial Sequence
  Probe  1418566_s_at
aatgttgagtaaatttctgagtaagagttgtataccctttgtctaaatatgcttgtctgttcacggaaaggacagaactggaaattttaagaaaggaaaaaaagtacacntgaagtactttggataacttgagttaatgaaaagttnctaactggtttggtgctgcctaagtgattatntcaagaaattgaaaaatgataaacccctaacaacggaagagttaattgcctcttaatngtcagacaacagttgaggcccctga

SEQ ID NO: 55 - DNA, 211 bp, Artificial Sequence
  Probe  1418571_at
cccaggtgtggcactgatccagtgaggagcacccgcgctggggcccactcgtcgtccattcattcattctggagtcagcctggccttccagacagaagccgagccagactctttcaaccacaagggggtggggcgaggtggtgattcacctccaaggactgggcttagagttcagggagccttccaaggtgtccaattgccctatctctgg

SEQ ID NO: 56 - DNA, 500 bp, Artificial Sequence
  Probe  1421948_a_at
gggtcctcaacaaaatcctgaaaggcaacattcgcctgggcaagctggaggagagggtaaaggggtacaagaagcaggcagcactgaagctgggggatatccatcaccgcctgaaggaacagcaggaggactttgccggcaaagcagctcagtaccagaaggaggtgaagcacctacatcggatgctgcaggaaaagcaggaggtcctggatgaggcgctgcagcagaaaagaaatatggaaggtgagcttgaaatggtcctggagtccactgccaaggagaacaggaggatgcggagccttctccaggccaccctggagagaagaagcacacagcacgtcacagctcctccggacacttgcttgagacgctcctctcagggagacctactgattggccatgatttcagctatggtgatnnnnnnnntnnnncnaccaccaaccgccagagtctgggggaatctatggcgtagatctcctgtctgaggtgtctacttcct

SEQ ID NO: 57 - DNA, 270 bp, Artificial Sequence
  Probe  1422198_a_at
gtacatcaactatgaccagctggaggagaatgccagcctcttccacccgaagctgatcatcgcagggaccagctgctactcccggaacctggactacgcacggctgcggaagattgcggatgataatggggcgtatctcatggcagacatggcacatatcagtgggctcgtggcagctggggtggtgccctccccctttgaacactgccatgtagtgaccactacaacccacaagaccctgagaggctgccgtgctggtatgatattcta

SEQ ID NO: 58 - DNA, 304 bp, Artificial Sequence
  Probe  1422483_a_at
ttcgagtccgaacgttcgtggtgttgaccagcccggaacgaattaaaaatgggtgatgttgaaaaaggcaagaagatttttgttcagaagtgtgcccagtgccacactgtggaaaagggaggcaagcataagactggaccaaatctccacggtctgttcgggcggaagacaggccaggctgctggattctcttacacagatgccaacaagaacaaaggcatcacctggggagaggataccctgatggagtatttggagaatcccaaaaagtacatccctggaacaaaaatgatcttcgctggaa

SEQ ID NO: 59 - DNA, 468 bp, Artificial Sequence
  Probe  1422484_at
atgtcacctcaaacctactaaatggttttatacttagatttatataactgggcatatgaatatgcttaaacactgggaaaattctatcactgtctcagaaacaagaagactcaaatgtgtttcagtttgtgttcactggcctctttcaggtcatggctaaccaccaggaggcaactgtctattcttgacagtgcagttttaattagaattccctacatcaaggatgttgccttttactattgaaaggcatttactgtggtttatgtatgatatcaaataaagagtatttaacacttctttatagttgatntgcaaattaaaatgcttttagaaacaagcaagttaggccttttaaattntctattttctaagactaagttgtaaatcagaattgtccttaaatacccactcagaaacacacataactggaattcgattaggaatggcactctttgctaacttactaga

SEQ ID NO: 60 - DNA, 551 bp, Artificial Sequence
  Probe  1422767_at
taagaagtacgcattgccctaccgcgtgctcgacgctctggtcttccactttctggccttccggacggaaaagcgccagctgcctgtcctctggcaccagtgcctgctgacactagcgcagcgctataaggctgacctggccacagagcagaaggaggccctcctggagctgcttcggctgcagccccacccgcagctgtcccctgagatcaggcgtgagcttcagagcgctgtccccagagacgtggaggatggtggtgtcaccatggaatgagaacagtcacctgtgttggcccagggggcgcaggaccaccccaggaccccagctggtatagttaaagacctaggtcaagacactgaagggctactatggctgccgtggcctaatcctgggcagggaggactgcatctggttggctttctctgccattttaggctttggtccctgctcacttctggaagctaaccggagttacctcagttcggttcagcgaccccacaacccacctggcagttggtataaggtctgctctctccagatactgtgtaag

SEQ ID NO: 61 - DNA, 172 bp, Artificial Sequence
  Probe  1422844_a_at
tgggaatacttcatactactcagaggtgcgtaacttatttttatatagagtttaattcactattatgggaattacttccatatacaaaagtctagcccactgtaccttgagaagacaaaacagttttgtacaagtccctgttatactgagtcaaatccattttctgggtgaa

SEQ ID NO: 62 - DNA, 501 bp, Artificial Sequence
  Probe  1423138_at
tccctggttcatccctagggagggagatcacggcagctcagtccctaccactagctcaacagtccctgatctccagagtctgcctttgtcactgagaacaggnctcctgccgggttgcagggaaggcaggtggtgttagcttagctaagagtctagagctgtgtggaagcaccttcacattgaaggatacactggcccacagcaccangcatgtctgtcngggcccacagtgtcgtccctggggagctctagggagacctgcaggacaggacctgccttcacatcattgccggggnctctccaggtggagctctgtatggctctgtagctgcttgctgttttgaggcagattcagttggtgctgttcgcactcctgggctgggctgagcagactgagcaagctttggggctctgagggtgtgaccttgccaggccagccnttgttttacagggaatgatggtgacctcgtggttctgttgctctgaactctgcatacactg

SEQ ID NO: 63 - DNA, 152 bp, Artificial Sequence
  Probe  1423160_at
ctgttaagcactagtcactatagtgcatctcctggttctgtactgtattttatacgcaacgtacatgctttaatattttaatgtttgtgcattaatattttcagtttgtttaaccactttgctgctaagattttgccatcccgttcccattt

SEQ ID NO: 64 - DNA, 201 bp, Artificial Sequence
  Probe  1423161_s_at
ctaaatccaaaaccagacactgtcgcagcaattttcagtcttatttttactgtttctacattggaggctgctgcacagatagatgatcttcgtccttgctgttttcagaggagcttggtttcctagagcagactgttacctttcaggattcctttttcttgctgaagtagagtcctggttttccttatgagtgattatcca

SEQ ID NO: 65 - DNA, 452 bp, Artificial Sequence
  Probe  1444235_at
aactcttggtgtgttggatacccacttgaacagccactactcttgcncaaatcagagtaccncaatnncatcccaaaccactntttnnccctccncctncagaangaaaaagtcacagagcaatcccccnagagctttnannctnagaantctnagacaaactcacacagcntaaatttaagtaaccgagagccctggcaggggaggcgcccctagacagtagtggctaccggtgctaagaacaagcacaccgtacaaagccagccattgtccgtcttccctccctgggtctcccgaaaaggacgttgtcattatcccaacgctctcctgaatggaattctttgaaccctcaaagtgattaggccataccaatcccatggcaccagtggactgtggcttgaatattatttttgatttgactatcaaaatgttctgatgattctgttccattt

SEQ ID NO: 66 - DNA, 360 bp, Artificial Sequence
  Probe  1423291_s_at
gggttcaacttgacagtgtacttcgctaggaagganaggcccttgcctgctataaccatgcctagagtgcctgtcctccagtattgcacctgtanccggagctagggccaccccagtgcaggggcaaancaaacctgatgtcagtcgttttgtnttttntaccccaacctcaccctntcaccctactttccaccccccaccccnccnttttttttttttttttttttttnnnnngtgccacattggcagaggggcctataggtccttcactgttccctcctgtgtgttggagttctttagttagcagttcaggctggttggacaggtttgaatcaaattgtactttgttccattgttaat

SEQ ID NO: 67 - DNA, 182 bp, Artificial Sequence
  Probe  1423480_at
gaagctgcaaaacatctctgatgagtggatctaaggagaatgtatatcagtctgaagctgttcccagtcatctgtatcatggagcactcagggcaccaagccagcatactgcattgtgcctactgccatttcttcatctttcatcttctaccatctcttctcctttctctcgggagttgaga

SEQ ID NO: 68 - DNA, 552 bp, Artificial Sequence
  Probe  1423586_at
gtctttggaccgagatgcttgtttctaggtctgcagcgctgttgctatagacaggcccactgctcgaaggctctgagattctatggctctagatttttctggctctataattcgtggcaatgctcccatggttttaggttgcacgactctgagattccaggacctaaggcttctagactttatttttctggagccaggggtcctgtcagtggaagattgtagatttttaaattctaaagattctaggcatgaaggttctaaggcatactgcttctccagtttaacagtttagggctcatgttggaatactccagatcataatgtttcaaacttttattttttttaatttctaagaccccagtgatggtcaactacagattctgaagccttatgaccatagattcttttatataaaaatcctgtatctcaaggaaatatgattctagactctgaaattccaaagctttaagagtctccagatggagtttctaagctatgatgtggtgataatctaaagtttagtccaaggttctagattcctaagcttccacg

SEQ ID NO: 69 - DNA, 551 bp, Artificial Sequence
  Probe  1423703_at
aacaggcagagaacttgcagcgcaagcaggagctgcgcgaggcccacaagaagaagagcctggcaggcataaagcgagcccgtgcaagggccgacggtgacagtgatgctgaggacccaggggcaccgccagaggcagtgggggcaggtcagcctgaagatgaagaggatgacgctgagtatttncgccaggctgtgggggaggagcctgacgaagacttgttccccacagcggccaagcggagacggcnggggggccctctgggcaagaagcagcgagggaaggagcagaggcctggtaataaaggccgaggtcagggtggcaactggcaggctctgaagctacaaggtanatcccagagagncaagnccaanccgagacccaganccacacaccaggactcacncccagcatcaaggagaagaaactgaacctnncngnnnntgggtggactatggatgccccagattggggcccggaagctgcctgggatccagtgcantccggtttccttttataaaggaactgcccagttcttaacctccaataaa

SEQ ID NO: 70 - DNA, 256 bp, Artificial Sequence
  Probe  1423705_at
gatgtttatcaaattgtttaccaagtggaaagctagttctgagaaattattatccagagcctgtgttattttaaagactggacttgcttcaaaggtacccatacgaggagcccaccgtccctgtccctcgaggtcacgtcccaccgctgcactaaggactcacggcccagcgtccacaagactgaggccctgtccctgctcagagaacagatccggagcaggtggtctggtcagtcaggatggaacagtgctacaa

SEQ ID NO: 71 - DNA, 522 bp, Artificial Sequence
  Probe  1423730_at
tctacctttaccagcggtggctttaccctgtggataagagcagggtcaatgaatttggggagtcttatgaggagcagcccaagcggaagccccnatccagactgagcatctcggcccaggctcccaggcggcactgtactgcacagcacagttgggccatgagtattcctgggggcttcgggaaaattcctagataccacatcttctgcgttgccaaatccctctgagcatttctcagtatccctgagcaccctctcttctcgtgtttcagaaggaaccatagcacttaaccatacaccccacatgaacttgggtcttggtgattgtgccnagtgggcagtcacccaagggtagctctcagtgcatgagtntcccatgncanccagtggacaccttagcacncaagggtagggagcatggtggatttccacntcagcttgtccaagtgctccttatgtggatttcacatacacaaaantcgttctgtacggactctgggatcaaatggctctttttcccttt

SEQ ID NO: 72 - DNA, 227 bp, Artificial Sequence
  Probe  1423815_at
gaaagctttcgttatcgctgcagggatgccatgcgctcagtgaccaagcaggccattcgagaggcgagactgaaggagatcaaggaggagcttctgcactcagagaagctcaagacatactttgaagacaaccccagagaccttcagctacttcgccatgatctgcccttgcacccggcagtggtgaagcctcatttgggccatgtccctgactatttggttcctgc

SEQ ID NO: 73 - DNA, 454 bp, Artificial Sequence
  Probe  1423823_at
actctaactgtgagcctggatccagggcccctgaagcaattccctcactccatggagccacagctgaggcagctggggctgcccactgccctcaagaaaggtgtggtgaccctgctgtctgactatgaagtatgcaaagagggtgangtgctgaccccagagcaggcccgtatcctgaagctgtttgggtatgagatggctgaattcaaagtgatcatcaaatacatgtgggacgcccagtcagggacgattccagcagatggacgatgacctgcctgagagcgcgcctgagtctgagggagagtctgaggaggaggatgaagctgantntgagagccaggactatgnccttccagcgagttnttnnnatnttnntgctggacaccccaccccacctcaccctgcccccggactgctntcaccantntagagggaacagctatttattttgcta

SEQ ID NO: 74 - DNA, 517 bp, Artificial Sequence
  Probe  1423841_at
tccaggactagacctaggttagactgagtatgcctcggagacaacgatgacatcaggagaatacaagttactttgacatctgatcatttcagtgcctctacctctggtgcatccaagtcttaggttttgaaaaacaagacctgtttgtaagcagttttaaatntatcttagacaacttcagattctagagancagtagcaacagggnaaacagcaacctgagccaccagtgatgatggggatagctgactagggagatgcatgaacatagnngatnnatatatatatatatntatatatatatatatatatatnanacacacacacacacccatatatacatatatgtatgtgtatatatatatgtgcgtatatgtgttgtatcagatggttttntcacatcttttcacctaacagtgtatgttttttcacatctgcagcatgtttattaactagacatatatgtgttacccattcagagatagttcagtttttgtgtttctagtatggtgcctgtg

SEQ ID NO: 75 - DNA, 499 bp, Artificial Sequence
  Probe  1423884_at
aacaacctggtcattgctcattctgaccagcaggtatttgaattcagcatcccagaaaagcagtatacagagtggagccgctctctccagaagcaaggatttcaccagctgtggctcncaaagggatactcccatcacacacatcagctttcatcccaagagaccaatgcacatcctcctccatgatgcctacatgttctgcatcattgacaagtcattgccacttccaaatgagaaaactgtgctttataatccacttcctccgaaaaatgaatcagatgtcttcttgaggcggacaacccatgggtttaaaatgtcaaagatatataagcccttgctctttatggatcttttggatgaaaggacacttgtggcagtagagcgacccctggacgacatcattgctcaactcccaccacccatcaaaaagaagaaatttggaacttaaaacagggcactgtctgtgtcctttcttgaactgtctatcctgttgttttgt

SEQ ID NO: 76 - DNA, 372 bp, Artificial Sequence
  Probe  1423909_at
tgaggacccttcttgtgctggcaagtttctgcaccgctgtggctgccatcgttattgggtctcgtgagttgaatttttactggtattttctcggagatgatgtctgtcaaagagactcttcatatggatggtccaccatgcctagaaccactccagttcccgaagaagctgataggattgccttgtgcatatactacacaagcatgctaaagaccctgctcatgagcctccaagctatgctcttgggtatctgggtgctgctgctcctggcttctctcacccctgtatgtgtctacatctggnaaaagatttttcacaaaggcggaaacagaggagaagaaactgctgggtgcagctgtgatctagcctttc

SEQ ID NO: 77 - DNA, 464 bp, Artificial Sequence
  Probe  1424001_at
aaagccactgggaacctgactgtgccctttcctgtcctgaatttgtcttcaggcttcatcggtccctcagttttactatccttatgaaactcaggcttaactaccaccaaacaaccgattggtgctgtttggacggtgtgtagttgtgtatctaaacccctccaaaatatgtcaggcttcagcctcagacatctgaaactggaaatgaaatccagagagtgagagggcagttctggtttagatttccacatgtgtccaggagctgaggaggantccgaaatgacccacagttactgcagctaagctagtgtgtaggtntcacaaatcagtttcagcagttgtattcaagatcactgaccagacttttctacagtttatttgtgtacaagagaacctaagaactttgtggggaagtaagaaatgtcaccantctcctgttgtaagtttctgtaccctttctatgc

SEQ ID NO: 78 - DNA, 507 bp, Artificial Sequence
  Probe  1424019_at
tctgtcccagatgcccaagtgtctaccaggcccagtcagtctgctgggaatgctgacgtgaacagcaaacgaaagaggtctgagaagctgaagcagcggggacctaagtggaagccctctaaggaggctgctgtcccaaagccgagcgccccttccagggttgaggactcgagaacacctcctgtgccgactccctctgaaatccgtgctgccccaaggcctaaggactgtgctccatcccttgggnaagccaaaaagaaacagaaaggaaagcagcagttagcacagcagcctgctaatggagctgctcccctgaaagaagatgctgtttccaagggaccctcagccccctttgtgtctccccacagttccaccaggcccccaccagcaaaaaggaggaaatcaatgacaaagggcaacagccagcctctgttgtcttaaatggacttggaaacttgacattgtctctgggttggaacttttagatcctgatgcaaggctggcctc

SEQ ID NO: 79 - DNA, 263 bp, Artificial Sequence
  Probe  1424151_at
ctgtcaagaatgtgcccgagaatcttgtcaagtgcttcggggagcaggctaggaagcagtcccgccacgagtatcagctgggcttcactctgatttggaagaatgtgcccaagacacagatgaagttcagtgtacaaaccatgtgccccattgaaggagaagggaacatcgcacgtttcctgttctctctgtttggccagaagcataatgctgtcaccctcaccctcatcgatagctgggtggatatcgccatgtttcagctt

SEQ ID NO: 80 - DNA, 516 bp, Artificial Sequence
  Probe  1424227_at
tggtggacgagcaactagccctggagctcacagtgactgcccagcctgaagaccctgtccagacttgggaaagtagagagcagtttgagtgttctccagagccaagcttggtgggaccaggagcagtgacaagtgcttgtcctctccctccccagcactgcttctctggtctggagccaccctgaatcgtctccagagtgtcatggatccaggctggcttctcacttgctgtatagcatagagtagcctggagtttctgatcctccagctgggattccaggtgtgcagtgccaccttgagcctatgtggaaggtagggatgggtcccaggactcgtgcacaccagggaggcattctcccaggggagctgcttccccatcacaaggactgtttggtggtttttgtaatttcttcagggctgaggatggaacccagggcctctttcatgttaggtgggtgttgtactactgagctaggcccagtctatcctatgaacttctggctttcagtgacgcag

SEQ ID NO: 81 - DNA, 435 bp, Artificial Sequence
  Probe  1424244_at
tgaacgccttttttaacaacaccatctcctcggcagtgaagcagagcatcttagccaagctgcaggaagcagtggaagtgaatctcggcacggccatgacctacacgttgttcgagtacgctaaggaccataaagagcagttcatggagaaccaccatcctgggaattctgcaacgcctgtagccaatatcatctcggttgaaactcccaccacagccccatcaagtaagaaaaaagagaagaaagaacaactttccaaagctcagaaacgcaagctggcagataaaacnagatcacaaaggggagctgcctcgcggctggaactgggttgacgtcgtgaagcatttaagcaaaactggctctaaagatgatgagtagcaagcacctgccgcccaagcaagagcagcctttgaaaagcggcctggttcagcatct

SEQ ID NO: 82 - DNA, 571 bp, Artificial Sequence
  Probe  1424343_a_at
acttttgtacttatggagccatgccattttatactttcacgttgtatacattcgtttgatcctttaagttgttgccacccataaaaaggcatcttacagtgcagtttttaaattacatgggtagcaattttgagttttaaaaattagtcattgcagaaattaaatacttagaggagataatccattatcttgactttaggaatataatagttgacaatgtttatatataattttacttctctaaggcatacccaaaaatagaaaatgaaaanagagcagtgagtctgttctgatgcttgcattgcatagagaagttttccaacaaagcagctgttaataacacataaaatatgttttactttgcaaagtaggttgtgttaagtcattttcaaaaagttacctactantatcgaggctctggataattactatgtgttgattaaagttagttacagaattgtacaagctaagttttccttaaactaagcttaggttaaagggagaggagccacagctcaatgaaaacacggttcctgttttctaaatggaggcgcccagaaacacaataaaa

SEQ ID NO: 83 - DNA, 419 bp, Artificial Sequence
  Probe  1424344_s_at
cacacttagaagcagatcccacatccccagaaggacagtgtgtgtttagaaagaacacatcactggagctttttattgctctacacagtgtatctaaataagctgtcaactacaatttatcctattgctgctgtaaatttttatgacagaaagaaaacctgaccatggaccagctagcttgatggccttcagcagcaaacaagaaactgtccaagttaggaggtgaggactagtgcctgaagatgtcctctcagtccacaacatgtacaggtgcccatacacacatcagcactcgcacaaagatgctctggaggctatagtagtgtgtcttggtcattgcaaaccatcagaggcaaaccctgaggtattcccatttcctgtttcctgcttgcagtgtctacatttctctcccattctaa

SEQ ID NO: 84 - DNA, 509 bp, Artificial Sequence
  Probe  1424436_at
gaaaggactcgctgctctaaagtttgagggagccatctatagaaaggacattggcttccgtgctgtggccttcctccagcggcccaggtaaagatgccctctgaganggttgggtgcagggtcagaactggcctccntggctgggaggaaactcggctagnnccngttttaagtcattgcgtatttcctgcctgtggaactcactggaccttcgcttatattttggttgaaatctaaactatttntaggctgaggtaggaagggcatctcaaattcaaggataagtaagactatcacccaccacacacacacncacacgcacaaacttgatcaaagcaatatttgaataaataatgaaaaatcttcttggcaattccttatctttgaaatttttgtcaagataataaagacggttactccttgagttattacataattaaatgtttttgagcttgtatcagaggccctggtgtgtccctttatntactacctgagtgtttttaatccct

SEQ ID NO: 85 - DNA, 360 bp, Artificial Sequence
  Probe  1424473_at
ttcagatttaccctgtggacttaggtgacaagttccggttggttatagctagtactttgtatgaagatggtaccttggatgatggtgaatacaaccccacagatgacaggccttccagggctgaccagtttgagtatgtcatgtatgggaaagtgtacaggatcgaaggggatgagacctccactgaagcagccacacgcctctctgcgtacgtgtcctatgggggcctgctcatgaggcttcagggtgatgccaacaacctgcatggatttgaggtggactccagagtttatctgctgatgaagaagctggccttntgaacttcgtgggaaaccagacttgctgcttagtcactcatgt

SEQ ID NO: 86 - DNA, 504 bp, Artificial Sequence
  Probe  1424522_at
gggtaaccaagtacctggtgccctgcatcgcacagttctctgtggccatggcagatgactccatgtggaaacccctgaactaccagattttgctgaagacaagagactcttcccctaaggtgcggtttgcagccttgattactgtcttggcactggctgagaagctgagagagaattacattgttctgctaccagagtccattcctttcctagcagagctgatggaagacgagtgtgaagaagtggagcatcagtgccagaagaccatccagcagctggaggccgtgctgggagagccgctgcagagctacttctgaggcagaaagcgcatctgcccaccgcctgtgctctgggaagaagtcacactcattctcatgttttaatgggggtgtggtgccatgttactttcggtactttcacatctgccgatacttaatgtttttacaaaatccctgcctgtctgggataccacagaatcctctcctgccttttgtacagagttgc

SEQ ID NO: 87 - DNA, 498 bp, Artificial Sequence
  Probe  1424545_at
tcgtggctctggttgtatggtgctaccagagggaggagagcggcatggacctgtggttgaggcaggtgttggaagaggaggacgaggaggagccggaaggtcctccggaggagcttgaagctccggccctctacggggctagaacgtaacagggtgcgtgctgaggaagggactaggcagccgtcctttcgttgttgaatttgacnatttctgaactggttctgcgcatgtcctcgttgcttaaaggacttggatggcgctcttggcgctgagacacaagaacacagcacaaagttggaggcttgaaatctgatctttgccgaacaccaatgcccagtgattacgtccatagaacttttgaggttcctagaaaaggttttgggtgcaccttatgctcttgagttatgatgtgttcatacagcctaatcatgtttccagcgttcatctnggtggaggtgttacctgcagttcattcgctttccgagacagactaactgc

SEQ ID NO: 88 - DNA, 174 bp, Artificial Sequence
  Probe  1445562_at
ttccacctttgttgcactcttcctcctcctccattcctnttncttatttaggtggggnaacttgagggggtgggaaaccgatataatcacctctacccncagcgtgctccggaagcttcctgaccggccgagctgagctgtaccttatggaaagtggtactcagctaaaaaagt

SEQ ID NO: 89 - DNA, 346 bp, Artificial Sequence
  Probe  1424620_at
atggcccgggatgagaagaattactaccaagacaccccaaaacagatccggaataagatcaatgtctataagcgcttttacccaacggagtggcaagccttcatcgattctctgcagagtaagaagatggaggtagactgagtggggcacacccccagacctaggtctgctggaacaacgaaaccaaagccaggctgtaaggcaaggggcgtggagcatcagtgatgctggctaagtcctgtggaatcgggtcttacgtgccccctcccccttccttctggagaaggaactcttcattttgttttgttgttttttgagaaagagcatctctctaatagccttagct

SEQ ID NO: 90 - DNA, 182 bp, Artificial Sequence
  Probe  1424621_at
ggattttgaggctccttagatctcctgaggctccactgaaataacagtcaaggagcttggttttatgtgcattcatattcatgtcacttgcatatgtggacagaagtttcaacttctaancaaagtgaacatgaaatatttagtgtgctttccaacatggagattacttttctattttctaa

SEQ ID NO: 91 - DNA, 156 bp, Artificial Sequence
  Probe  1424724_a_at
ggtttattggaagacgcggatgcattgtggactcacagtcacagccaggcacagaaaacagaagagacaagctctggtccggaggggactatccagacccagaatccacattacagccgtgaagaattgaattcgatgactcttgctgaagtagta

SEQ ID NO: 92 - DNA, 228 bp, Artificial Sequence
  Probe  1424843_a_at
tgaatgtattgttccctgagagcaattgtagatagaggagtgggtgggaagtctgaagtcagggcattgtttctgcgtatctgaggaggctggtttttgatatgtgggagctagtggtcgttaacgcatttaccccctatttggtggactctggttcccccatcttaagtgctgagtcaggggcctaaccgggcagctttaactatttgtttgttgtaggtgctagaa

SEQ ID NO: 93 - DNA, 275 bp, Artificial Sequence
  Probe  1424906_at
cagaatattaagtagtcttcccacaatgagtggggtctgctcagtaacaagtggaactgttcacacctgggcagtttanctcttccataaacataatgcagtataacatcctctcaagctaagcagggctagcaaagtatgcagagatggaactgggttcatataccaagcagtcgtccaacacagtgtgccttactaaacagagccccatctgtttagacgaggaaatctcaactgtctctctatttaagcacaatcccaccattttttaaagg

SEQ ID NO: 94 - DNA, 414 bp, Artificial Sequence
  Probe  1424942_a_at
gaggcggacacacaacgtcttggaacgtcagaggaggaacgagctgaagcgcagcttttttgccctgcgtgaccagatccctgaattggaaaacaacgaaaaggcccccaaggtagntgatcctcnaaaaagccaccgcctacatcctgtccattcaagncagacgagcacaagctcacctctgaaaaggacttattgaggaaacgacgagaacagttgaaacacaaactcgaacagcttcgaaactctggtgcataaactgacctaactcgaggaggangctggaatctctcgtgagagtaaggagaacggttccttctgacagaactgatgcgctggaattaaaatgcatgctcaaagcctaacctcacaaccttggctggggctttgggactgtaagcttcagccataatt

SEQ ID NO: 95 - DNA, 420 bp, Artificial Sequence
  Probe  1424988_at
agagagccactcagtatgcccgagtggttcttcactttcccaagcactcactccagctgcacccatgggtgtcgccttgcttgaagatcaaactttctacagccttataggtttctagatagntgtctcctttttgtgtatgtcttgtttctcgttgttcgagttttcctatgtcagtgcttccatactcattgtcctgccccctcggtgtcttccagaggtagggctacttctttatgtttccatattctaagttttcacccccacttgggcattttggangctagtgagctagggggttttctagggtgtcaggaaacctagctgacctcatcgggtgcaatactagctaagttaaagctagaagcctacactgtcactttactgagatttctgagtctacgtttcatattgccttaa

SEQ ID NO: 96 - DNA, 416 bp, Artificial Sequence
  Probe  1425177_at
attctcagtcgcatggtgaatcacaaccacctgtaacttcagttccagagaatcaatgccctcttctgggttctgaggacaccgggcacacatgaagtacaggcatacatcaggcagaatggtgatacacagagaattttgttttaaagaaaaaaanttacagtcagcaacttaatgtaatagaagggctagggagtgtcacttagagataatatacatattccgtatttgtgacgccctgggttcatgcctaggacacaaagacaannnnnnnnnnnnnnnttggctgtctccatgggtatgaatcccctcctggtttaagaatcagcagaggccaaaggctttatattccacctgcctgagataagcagctccttcctacaattaggaacccagatgccaccttctgaaagagt

SEQ ID NO: 97 - DNA, 401 bp, Artificial Sequence
  Probe  1425178_s_at
aggcatacatcaggcagaatggtgatacacagagaattttgttttaaagaaaaaaanttacagtcagcaacttaatgtaatagaagggctagggagtgtcacttagagataatatacatattccgtatttgtgacgccctgggttcatgcctaggacacaaagacaannnnnnnnnnnnnnnttggctgtctccatgggtatgaatcccctcctggtttaagaatcagcagaggccaaaggctttatattccacctgcctgagataagcagctccttcctacaattaggaacccagatgccaccttctgaaagagtctttatttgtacatngctgaattgcaaaccatcagtaactttttaatatggacaatcaaatttttacccacagaatgtctacaag

SEQ ID NO: 98 - DNA, 545 bp, Artificial Sequence
  Probe  1425179_at
tagaagcctgttctatcgcctgcaacaagaatacctgcccaggtgacaagagcgcgttacggcccagtggactgcgactggggactccagcattgacatcccgaggacttttggaagaagacttccaaaaagtagcacactttatccacagggggatagagttgacactgcagattcagagccacatggccacgaaggccacactgaaagagttcaaagagaagctggcaggagatgagaagatccagagtgctgtggcaactctccgggaagaagtggagaactttgcttccaacttctcactacctggccttcctgacttctgacaggaacagcctgtgtttgccaagagggtaggattgttccgggaccctgtgaaccctgtgccctcttgaggctgagaagtggaccactgagctgtaccatgctttcntttggtgnggtgtttctgtttacctgttttcaaatattttgggaacaggttattaaggacttgaaatttgaaacgttatgtagccattagatctcctcgctcatgcatgtga

SEQ ID NO: 99 - DNA, 566 bp, Artificial Sequence
  Probe  1446598_at
taatccctgacttgctcatgctgtacattggatgtaggtatatttgacaggatggtgcatagtaagaggcctccccattatctgacgggctcatgtttgccttgtatttctgatacgcttagctgcgctgtttggatgggcctttgccagtggcatatactgtttggggaggctgttcacagcatatcttatgcattttgtacctgaacagcaccactgaacaggattcaggtgaattcctggtcatgtctgtacctcaactgggagggagtcagccaaaatgcttacagacgcnctggtatttgccagtatgtctggaagggcacccaaggaaggagttaggtaacttgtgaatatgacttgtccagaatgggcagatctccaagtctgagaatttataaaatttgtctcattcaaagatctgagagcaattgatacctgtcaacactgggaagcagccatggaagccagggtgcaaatgatagccgagcagaagcagaaccctacagcagctagccgctggccgtgggtaggagtccgagttgtcattgccttactgtgaccat

SEQ ID NO: 100 - DNA, 187 bp, Artificial Sequence
  Probe  1425603_at
caaaatttccaagatcctgcttttttgtttttcttaaagttactttcaaaggggaaattggtatttctcatttctgtcttgcatgcttgtacagattgcctgcataatgcaggcgacctaaccagacttcaggatgttctctggtttcctgtcctacatctttgctgtccttgcctgcagtgtgacc

SEQ ID NO: 101 - DNA, 483 bp, Artificial Sequence
  Probe  1425684_at
gccacatgtgtggagcatcacgtgcttatgtttccttacagttggtttgagaaaaactgattctggccagctcagacaacctnccaactggcttgtcnacttcttattcattccctggacagaggggataggaatctcttgggacaatcttgcataacacacctcaaangatcagctctcctgtcaatgatagcaccacctcactgntgggtagcaactnggancaaacccccaaatgttggcagatattcattacagtctgaagaacttaaagctcaataccnggggccctgctcctgcctgctcctcagagttgaaaggggttagtcagggtccgcagagatgactgtgcagttgagatcantgactctcttctagaggattctgtttgactcctagtacccgcatggaggtttataaccatctgtaattccagttccaaaggatccaacaccntcttcttgacctccacagtaactagac

SEQ ID NO: 102 - DNA, 533 bp, Artificial Sequence
  Probe  1446771_at
tacttccctgtcctacatagacgggcccctgattccagcacccaggcattgtgaccagacacaaggacactgaacatacagggaactccctctggaatagaatatttcattactgaaggatgaaantgaacagntcacgnacatggggttaagaaacttaattctagtttagactgagttgccctaagacagagcctnctgnttggaagaataggaccacaccancaaaaacccantctgggaagatgctgcagagagatgggatggacagacgcttgtgtgatggatccacagcacagccctgacctcgtntggatcgatcacattcgttcatcaagagtttgagtatctctatccctttccctctcggntgtgttccaagaccctcactagatgacgaccaccaagagtcctgtccctgtacctatgtctgtctgtctgtctgtctgtgtttttctatacttacacacattatgaagtataatttagacaccagcacagtgagacattagcaatcaccatcaacagactgt

SEQ ID NO: 103 - DNA, 462 bp, Artificial Sequence
  Probe  1425820_x_at
ctgctgggacacacggacaggagcttcagggacagcaagaagcgagggacgtgtggggaggacttagatacccaagaggaagaagggaaggatgncctancnaaaggagagagaaaggtcngaagaaaagncaaaaggaaaagncancagtgctgtgaggaggncttagatgtaancagtaaagatgatggtgggacatgggtancagaggatgcaggtgagagaagtcggcaatncccaaaggagagagccnaaaanaanaancggaanagggnccnagggtctgaagtggatctcagctgactccntttcaggganagcctggtttagggcagggtttntgcncnctctgcaancagctntggagccccagtntgcanagganagccaatgcnttctatactggctgagggggtggacatggtcccctgtgaaacttgctgtccaaaaagtataaacctg

SEQ ID NO: 104 - DNA, 512 bp, Artificial Sequence
  Probe  1425830_a_at
gggtgtcatgaagctccactgagtgacagaaacaagaacagctgagggcagttttataaatctgtggagacaacctgagtttcccatttcttttttgtacctatctacttttttttttnttttntgtggtgctggaaatcaaatcaaatcaagacttcacacaagctgaatattcaaccactggtccacactgccaaccctgtgctttgaagagaatgttttagttctgaaactaactatcccaggattattagatgttaaagtctcataagtagggacagagtggtgagttagcagttgagagcactgtggacttctggcctcccctggcagcaacacacaaattacttaggtatctgtgtatcggccgaatgtatagatgaccagggcaactgtaaatgggagtgggctccttggagttagaactacaagaggttgtcagccacctgatttgggtgttagaaaccaaactccagtcagtcctctggaagaacagttcacgctcttaactg

SEQ ID NO: 105 - DNA, 457 bp, Artificial Sequence
  Probe  1425837_a_at
ggtgactttgtgtgtacattcttcacaaataattcctgtttgctatgcctttgacttctctgttgtgttttaaagaaattccgtagcattcagttctttagtttccactgtttagtcactgctaggcacttgtgccttttctgttgtgatgacgttggtagacgtgtgaagattactccaatcacttctcctgggatgtgctttttataagtagctttaggaacagctctggaacagttgctaagaatattagtgagaaaccttgccaagacgacagacatgagcccaacattcagctgagacaagaaagctcacgtcttggaggccagcttgggctatatgtcaagaccctttcttgaaaaagccagatgactggattcaagttttctagaaaaatataagagtcaaaaagataacctggagttctctttcaattgttgaagcctcttccaaacaa

SEQ ID NO: 106 - DNA, 416 bp, Artificial Sequence
  Probe  1425921_a_at
aggccagtacctagtcaccattgatgccctgaccccatccttggtgaacaagatgttacttctggtggtgctccttgttggggtgaccctcttcatcgcagtcctggtgatgtttgccctgcaagcctatgagagctacaagaagaaggactacacgcaggtggactacctgatcaatggcatgtatgccgactcagagatgtgagggcttctcgggaggccagcctctaccatggccgcctgttttgcctcagaccaaactaagtgcttccagatcctttcggtgcaatttcagagctgtgccagatgctagacacatttaatagctgtcagatcagctcatgatcatatcatgagaaaaccgccttttcatatttatttttctaactgatcacatacgcaggagtggccatggt

SEQ ID NO: 107 - DNA, 331 bp, Artificial Sequence
  Probe  1447105_at
gagggtgctatgtttcactttttgtgttacatgctttgagggagattgcatgtatatatgtgtgcagatgcacacactcacaaatgtccatagagaggccagagaagatgcctttctctgtcgctttccaccttatgtccttgagagaagctttctccctgaacctggagctcgctcactgttgtccgctaggtgggcaggccagagagatccgaagtgttctgatccgaagtgttcgacattacaggcacacattgccagagccagactttacatggatgctgtgtcacttaatttaatccccacgatggcacagcagatacttgttgtt

SEQ ID NO: 108 - DNA, 225 bp, Artificial Sequence
  Probe  1447112_s_at
tcttgatccaatccctgtgcagcatcaagaagcccaggaagctggcccaggcctgctcctctgtgtttccccatgggcagcgatagaggccaagtatnttntgnnnactccnncctaaccctncattggganatcgaagcactgcttaggttctgaggtgtgggttccctgccacccagncanacaggctgaagnttctcaccttttctttccagtgtgattaca

SEQ ID NO: 109 - DNA, 248 bp, Artificial Sequence
  Probe  1426311_s_at
agttacatgacggtggtgggagatcaggaagagagcttggcctaatttaagaaacaccaggcaaccctgccctccattcttgggagacagggactgtgttgggcttcagtgagccccttcaatgcttccctataatcagaaaaatcttaaaatccaaggccaggagagaagaatccaaaaagcaatatttttcatcacatgccaaaaatgggatagagagaaggagtggcaggcctaggcccctccga

SEQ ID NO: 110 - DNA, 293 bp, Artificial Sequence
  Probe  1447403_a_at
tgcaaaggaggctctgttctctctggtaataggtgttctggagtgttccagtaggttggctacttgtcttagtttctattgctgtgataaagcatgagcaaaagcaacttgggaaggagggattatttcagcttactgtttgctcttcatatcttgctcagcctaacttcttgtagtgcccaggattgccagcccaggggtagcattgcccaaagtgagctaggctctcccaaatcaatacttatgaaaaatcaggacagagtagtagggccattttcataattgtcctctca

SEQ ID NO: 111 - DNA, 437 bp, Artificial Sequence
  Probe  1426381_at
gctcttaccgtgcaaatgaccattaccaaaggcagagagtgctacagaaggagcgntgcaatagaagagagaagggtggtcttcattgggaaaatacctggccgaatgactcggtcggagctgaaacagaggttctctgtttttggagaaattgaggagtgtaccattcattttcgtgtccaaggtgancaactatggtttcgtcacttaccgttatgctgaagaggcatttgcagccattgagagtggccacaagttgcggcaggcagacgagcagccctttgatctctgctttgggggccgaaggcagttctgcaagaggagttactctgaccttgactccaatcgggaagactttgatcctgctcctgtaaagagcaaattcgattcacttgactttgatacattgttgaaacaggcccagaagaacctcagga

SEQ ID NO: 112 - DNA, 118 bp, Artificial Sequence
  Probe  1426394_at
ccagcaccattcagtaatacagccttaaccgtacctccttgaactacttcataacttgtcgagaaaagcagtttgcagcaagggcatgtgggatgcacctagtattaaaattgctttg

SEQ ID NO: 113 - DNA, 485 bp, Artificial Sequence
  Probe  1426426_at
actcaacttttcttatgtggacaaatagaagagataaaaatttcaacttataatattttaatacaaaaatagatttttacccccataactncatccatgtgatntgggataaggagagcggcctttcctgctggttagtcaccttcagtgtcatcgccaggagaatctgcagagccctgctgcaccttcctgatgcagctcaccactgtgctgtcttcacaggatactgcaattttagccagaagaccctgaaacatgagagcaaacnggcattgatttctgtaaaacctgtttccccacagcctttattcttttgtcacatactcaaaagatgagagactgcatggccctaggggagggtgcctccatcagaacaggtgttaactganaactgtcccatgcttgtaagtagtgggctggctnncggaagaacctccttcattctagccaaaacaactccaggtggacctttggctggaaaacag

SEQ ID NO: 114 - DNA, 580 bp, Artificial Sequence
  Probe  1426519_at
gttgggcatcctgtaaacgcattcaagttaatgaaacgtctgaacactgagtggagtgagttggagaatctgatcctcaaggacatgtcggatggcttcatctctaacctaaccattcagaggcagtacttccctaatgatgaagatcaggttggagctgctaaagctctgtttcggctgcaagacacctacaatctggacacaaataccatctcaaaggggaatcttccaggagtgcaacataagtctttcctgacagccgaggactgctttgagctgggcaaagtggcctatacagaggcagattattaccacacagaactgtggatggagcaggctctgacgcagctggaggagggagagctntntactgtagataaagtntctnttctggattacctgagctntgcggtgtaccagcagggagacctggataaggcgcttntcctcaccaagaagcttcttgagctagatcctgaacatcagagagctaatggtaacttagtatattttgaatatataatgagtaaagaaaaagatgccaataangtcagcgtcgggcgaccaatctgatcagaaa

SEQ ID NO: 115 - DNA, 507 bp, Artificial Sequence
  Probe  1426533_at
gaagcggtaggtgcacgcctcattgctcatgctggcagcctcaccaacctggccaagatccagcgtccacagtacagatccttggggctgaaaaggccctgttcagagccctgaagacaaggggtaacacaccaaaatatggactcatttttcattccaccttcattggccgagcagctgcaaagaacaaaggccgtatctcccgatacctagcaaacaaatgcagtattgcctcaagaattgattgcttctctgaggtccccacaagtgtatttggggaaaaactccgagaacaagttgaggaacggctntctttctatgagactggagagatcccacggaagaacctggatntcatgaaggaagctgtggttcaggcagaggaagcggctgctgaaatcaccagaaagctggaaaaacaggagaagaagcgcttgaagaaggaaaagaagcgactggctgcactggccctggcctcgtcagaaaacagcagtaccccagaggagt

SEQ ID NO: 116 - DNA, 557 bp, Artificial Sequence
  Probe  1426554_a_at
ggttctcggaccacatcgagggacagctccctgtaccnaggagagttgagagatagtaactatagtttttcttaggtctttgtttactaagggtctgcttaggaagaattcagggaggaactgtgctgtggtgtgaccaatgaaaagctgcagtccagttttgtccctggaacctgtcctgcactcttcctgcagttaggtaactgggggcatagtcattccagtggagagtggaagtaacctcatggtgatttttaaccctaatttctctctgaccctaaaggagctgctggccctagaaggttgggatcaatcttgtcaagtctatgttacacttacttttacaaaaaaaagtatatatacatattatcagcacacaaaagcaactctgaggttgctagtgacatttgaaacagtcctgtgtggtcaccagaaaataagccattcctcatatcagtgggataaactccttgtacctcttgctgtgtttggatcccttggccttgttttgtagaggtgaagtacccctggatcatgtccgagtttttgtctttcac

SEQ ID NO: 117 - DNA, 206 bp, Artificial Sequence
  Probe  1426599_a_at
ccactcaattaatctttccttgcctgagaccagttggaagcactggagtgcagggaggagagggaagggccaggctgggctgccaggttctagtctcctgtgcactgagggccacacaaacaccatgagaaggacctcggaggctgagaacttaactgctgaagacacggacactcctgccctgctgtgtatagatggaagatatt

SEQ ID NO: 118 - DNA, 359 bp, Artificial Sequence
  Probe  1426600_at
cacactgtagtcttcaccttgattggcccagtgcctgagggtggggaccacgccctgtccagacacttgccttctttgccaagctaatctgtagggctggacctatggccaaggacacactaataccgaactctgagctaggaggctttaccgctggaggcggtagctgccacccacttccgcaggcctggacctcggcaccataggggtccggactccattttaggattcgcccattcctgtctcttcctacccaaccactcaattaatctttccttgcctgagaccagttggaagcactggagtgcagggaggagagggaagggccaggctgggctgccaggttctagtctcctgtg

SEQ ID NO: 119 - DNA, 483 bp, Artificial Sequence
  Probe  21426630_at
aactgtgtagtcttgctggtaactcagagaatgatgctgttcgacaccagcatcctgtactgctatgaggcctcccttgcgcatcagatcaaggatatcttaaaaccggaagtaatggaggagatcatgctggaaacacgccagaggcttttggaccaggagggatgaggccgagagctgaagnatgggactgggcaccccctgggccagttttgggagtgaaaactctgccatgtggtccccaaggagcatctgacaaacacattctcacctgcagtggggctgcagagccaggcctgtctttctgttctgagagaaaactagggggaggtaatggtgggttgggctcttgggtcccagtagttttaggtctttcagagcacagtgtcccggcctgatgctgacttaccttggaataacttggagaatttatgacttccaatcaccccacacaatgtaatgccccatgaaactgtcctggta

SEQ ID NO: 120 - DNA, 186 bp, Artificial Sequence
  Probe  1447738_s_at
gggtcagcaggaagaagacgacttacagcggatcntgcagctgtcantcacagagcactgagttgcctggccttgggacggctccccaggnccatgccttctgcctggttttgtaatttatttatttataaactntctgctgctgagcttggganctgggcactgaagataggctagcagtgagac

SEQ ID NO: 121 - DNA, 82 bp, Artificial Sequence
  Probe  144790l_x_at
tggcccttccctgggggataggtgttatccttcacaaggcctgggacaccctcaacaacaaaattcaaagctatggtagttt

SEQ ID NO: 122 - DNA, 195 bp, Artificial Sequence
  Probe  1426931_s_at
gacacactcaagaggtggtgctggataagtctgcatgtgtgctggtgtctgacatgctgggatctgctactggagatgttcagcctgctatggatgccatcgccagtttggcagcagcagaactgcatcctggtggcaaggacggagagcttcatgttgctgagcatcctgcaggacatctggttctcaaatggt

SEQ ID NO: 123 - DNA, 491 bp, Artificial Sequence
  Probe  1426939_at
aagagcaggtttcccagcatagtacctcctaggtgcagtgactatgaagacggctgacaagtgtgggtgtggttgtagaccagacttgtggaaaactggcaggtacatttgtggaaaggaccagaattccacagtggtaggctggctggattggtaatccagccaggataagcatacagttggaagagagaaaagatgtagacatctctacattccccaatgtccccaccctagcctccctcagaacaaccctcatcctgcccagaatttgcatcctcctgggaatgctccctgtttgaatacccttgagagtttgaaggacttgactaaaagtggtgaacttgagaaagttgggctgttttaagtccagggatgaggcttaagaccttaataacatataacattttnttgtttaatccatgatttaaccttggaatcagctttctgcaagctgagcttcaaaatttggcaagagagtagtttctgtaccc

SEQ ID NO: 124 - DNA, 314 bp, Artificial Sequence
  Probe  1426958_at
gaaattgcatgctcgttaacccttctgtaaatgagcaccaatcatgacagctatacagagtgaagtctcagatttctttctcaccagctttcgtttatgggcagagatgtactcttgtctgaggtggtttgagaccacatggcattacaatgtggacaggaagcaaatcaaagcattagtatgggcactcttgttttccttccaagttactgagtctgaggtcaggagtgcctgtgagcctgcttgagtcaaagggcaggaccaaatggttcagcctcactttgtccccttattctgttcttcctgtccagggt

SEQ ID NO: 125 - DNA, 502 bp, Artificial Sequence
  Probe  1448104_at
gacattccctactctttggagctaagattattaaagtaaccagccctggcagaattcagagcttcagtcactttaacaaaacacaactctgattttcttttgttttccatcctaaagctaagtagacatttatactttgtgactgcaatgcattttgttttcttataaaacacagttcacattaatttaatccaacaattttgggctgggttgaaaagtggttattatctaaatccaagtagaatttaatcacagactcctaatattttccttagatataaaggaagttactttaaaaaaattacaactaatccaaattgtgttatttataatcataatcaacagatttgaccagctacttgtgctcaaaagggtttcttgtttcttagatgaaatttatatttggttgcatttgggttaatatcattaaatgagaaaaccagcattttccctttttgtccttatatgtnctttgctgaattttgtgagcttctatactgat

SEQ ID NO: 126 - DNA, 391 bp, Artificial Sequence
  Probe  1448126_at
aggaggtatacgtggctgccttagtattctgttcctcatgattattttagaaactggattttaaaaagtcttttagagtttcacaacaaggagaagccacagtttgtggcatcctatagttttacattgtgtatttcttccttttaggaaaaacctacctagtacaagctaccacgtgcacagacagcccaatgagtgggccatttctccacaaccctggtgtaatacagagtccagccattaccattctcctgcgttactttgacatgggatttttttnttttttttnnntacattttggctgcagtnattggtggtagnatatactttgattggatcatctctacttctgtatttatttatttatttattactagacctcaaccacagc

SEQ ID NO: 127 - DNA, 354 bp, Artificial Sequence
  Probe  1448135_at
ggaagcctgactctgctgcttacattactctaatccctccatgtgtaaaggaggaagacactccctctgacaatgacagtggcatctgtatgagcccngagtcctacctgggctctccccagcatagcccctccacctccagggccccaccagacaatctgccttctccaggtggttcccgtgggtctcctcggcccaaaccttatgacccacctggagttagtttgacagctaaagtgaagactgagaaattggataagaagctgaaaaagatggagcaaaacaagacagcagccactaggtaccgccagaagaanngcgggnctgagcaggaggccctcactggcgagtgta

SEQ ID NO: 128 - DNA, 180 bp, Artificial Sequence
  Probe  1448140_at
gcgagcaggtgctcctgagcaatagcaatctccaggatgcctaggagggacctcaagcccacacacctcctcctccangcaactcctgtcctcagctcctcccacggcctcctcccacctctggnttcaggcgctctgaaaacactcacagggagaggcttgcagccgagcgagcgagcg

SEQ ID NO: 129 - DNA, 532 bp, Artificial Sequence
  Probe  1448171_at
gacatctgtgaataccgtccttattcctgtccttgtcctggtgcntcctgcaagtggcagggatccctggaagctgtgatgtcccatctcatgcatgcccacaagagtatcactacccttcagggagaggacatagtctttctagctacagacattaacctgccaggggctgtggactgggtgatgatgcagtcctgttttggccaccacttcatgctggtacttgaaaagcaagagaagtacgaaggccaccagcagttctttgccatcgtcctcctcattggcacccgaaagcaangctgagaactttgcctacagactggnagttngannatgggnaacnngnnngaganctgnacctgggnaggccacacctcggtccattcatgatggcgtgngctgcanccatcatgaacagtgactntcttgtttttgacacagccatagcacatntctttgcagataatgggaaccttggaatcaatgtcacgatatntacatgctntcagtgaggctccaggaggctttccta

SEQ ID NO: 130 - DNA, 89 bp, Artificial Sequence
  Probe  1448178_a_at
cagtctgtcccatctcagtttgcaaaaagcactgacacgtatctcttctattgtaagctttccatttagtttgcttccaatgattaaat

SEQ ID NO: 131 - DNA, 394 bp, Artificial Sequence
  Probe  1448230_at
gtcccagtctgatcgcgtagtggaataagtcccgcaccagcagctcttgtaaatttgtgaaaatgaattttatctttccttaaaaaaataatttttttaattcatcacactctcctcctttaccctgtagtttttgataaatgacaaaaacgagccagtcgtcattagagatcgctcttacttcaaaaggaaataaatacaaaaaaaacttgttgctggttcctaataggaaacacattttaacgattacgagagggaaaccgctgacgtagacaccgcgggtcagacatgtgggtagacgtcaggctgcagaggtgggtgattgtaactttattaccctgaaaagatttcaaatcacattcatgctgtgtacacatgaacagtgggcacaatg

SEQ ID NO: 132 - DNA, 536 bp, Artificial Sequence
  Probe  1448413_at
cgctcctacccggagtttgtgagagtgggggacttgccatgtgacagtgtggaagaccagctctccttggcaaccatgttatatgataaggggctgctactcaccaagacacctctagttccgagttagttttttgaggatggctggaaagagtgccgttgcgactctccactaccgttgcctctctttttaaagaggtcgggttcttaccttgctaagcatcagtatggttgtgcttacctttaggattttttcagtgtcgcagacttcaggctggagaagaacctttttanccnaggtgtaaaagtaaacacaggaattgaagctgagggggaaagttcgtggcagaagtgncccntgacccctntcctttcagagcaccagcttcattccctacagtntccagtgtaccgggtagcattggctggnccattctgctggagacattgggggttttatatggaagttgcttccttgggttctatttcatcagtttgcggggctgagctggggtcagtgtgctgcgcgttaagttt

SEQ ID NO: 133 - DNA, 415 bp, Artificial Sequence
  Probe  1448450_at
tacataagaacctcatggatccccaggcccagagcacagcagtcaggctgggcctgtgcatcccgtgcagaggagggggctgctgactgccgggggctccccactccataaagcgagaagactgacctgcgcggatggaagagacggggtgtgaggttatgggtgccgctcagcagaccatctctcctggaattgtggggagcaaggggaaacagatcccacacttaagacttcagagttgcttgctctagtgacaaagccttcagccactcagcactccctagtcttaaaaccttgccttcattcatccacgggacccttttagataactgaacaaggcctaggagtgtagctcagtggcgtgtgtgtgcctggctctgtaaagccccggcttcagtcctctacagggaagggt

SEQ ID NO: 134 - DNA, 233 bp, Artificial Sequence
  Probe  1448480_at
gtacgtacgccatgaagagacactgacttaggacggtcattccaagaacattttgctgtgtgagaggacttagctttgtttcctgtgtgcggccatcagattaaatctggacaacactgagatacgcaacttgggttcttatcaacagaaatggcgtgaaaacagggtctctggccctctagagtcaaaagcaggtggatcacaagtataaggccagctcaggctacttggag

SEQ ID NO: 135 - DNA, 476 bp, Artificial Sequence
  Probe  1448563_at
tcagtgtttccagccatttgaacctggacagatgcagctggggagggccagaggaagtggtctatctcaactgtaaggcccatcccctcttaaccatgaccagcggaagcaggtgtgtgtgaccagggcacggggtgaagaatctgcccatcaaggtgggtgggcctcatgtcgctgcccccaccagagtcctaaaacctggatggacttggacgggacaagaggcctggactgagatgtgagtcccatggaagacttncctntccaccccccacatcggtcctntcaaatacccaatgggattccagcttgaaggattgcatcctgcagggaatgagcacacctgccaaggacacgtgcgcctgccttcccgctccctctntntcagagattgccctttcttcccgagggctgtgggccagcgctccgaaggaagcaatcaaggggagaaaacacagtgtaagccgctgtcaata

SEQ ID NO: 136 - DNA, 562 bp, Artificial Sequence
  Probe  1448568_a_at
gccaggtttccattgattcattccaaggtcccttttctcctgggctatgaattcctgtacatatttctctacttttgtatcaggcctcaattccagtatgttttaatgttgtctgtgagataacttaggtgggttctttttaaacagccaggcagagccatttgatggcatgtactgctttgtcggcctcaccagcttcttccccaacatgcacagggatttaacaacatgtaactgaagcttccctccctcatagtctctcatagaaatagtcacggcactctgctccctgtcactagtggcaggttctgttgatgtgtgacaacttcttagagggccgagaatctttggcacagtggaaatataagtttgtagtaacctctttgcaaacagttcacggacatgttgctaagaagcagggagacaaagccccctggcggttgtggttattcttctgagatttctggcagtgtgggatgggtgaatgaagtggaatgtgaactttgggcaaattcaatgggacagccttccatgttcatctgtctacctcttaactgaataa

SEQ ID NO: 137 - DNA, 427 bp, Artificial Sequence
  Probe  1448617_at
agagacactgtagcactggtggcttgagatccactgccagctgctggtgtggttatttctcagagtactagcctagcaaatgtgagcccttgagtttagccccaaatactacaaaaaagaggtccaagtttaaatgttagtctcctaacaactgtcaaatcaatttctagcctctaaatcttgctacttccactctacaaagtcacataagagagaagctgatggaaatttttgagtcccattcattagataattgacatactcagtttccttttgaacacagtccttggtaataggaatcatacagaaatcttttatttctggaaaatattccaatttctttgtcttattgattttgttccatccatccatccagaaaagattattcccatcctattgttagtcagtctggtagccttgaattaca

SEQ ID NO: 138 - DNA, 433 bp, Artificial Sequence
  Probe  1448625_at
tcttctcagaggcactttagggcagtggggctgggcagcacttcatgggtcctcaggcagttggggctaactgcctcaggaaggcatcccactttggagggcttccatctttttgaggcactttgggacagggaaagtgggtaccattctctcaggccttatgacaattggggtaactacgccaagcaggacagaggctgctggggcagggtggccttcccctcccccggtgtacatattgtacctgtgtactattttgtatataccggggtagggacacccctgtatcatagtggaggttggagctggcaaatgggaagagcttctaataatcactttgggctgggaaccttatttattggtagtgttaggtcagagggcaggaggcggagacaaggttgtggcacctgctgatgcagctcctctttatttt

SEQ ID NO: 139 - DNA, 497 bp, Artificial Sequence
  Probe  1427578_a_at
gcaatgactatgtggccttggtccacccagacttggacagggagacagaagagatcntggctgatgtnctcaaggtggaagtcttcagacagacagttgctgaccaggtgctagtaggaagctactgtgtcttcagtaatcagggggggctggtgcacccntaaancttctatcgaggaccaggatgagttgtcctcccttcttcanngtcccccttgtggcaggcactgtgaaccgagggagtgaggtgattgctgctgggatggtggtgaacgattggtgtgctttctgtggtctggnacancgaccagcacggagctgtcagtggnngnnagnnnnntcttcaagctgaatgaagccaagccaagtaccattgccaccagcatgcgggattccctcattgacagcctcacatgagtcaccgtccatgctgccgtgtgggctcctgcctctggactttggctcattttccatgcctgagctagtctgtaccagat

SEQ ID NO: 140 - DNA, 426 bp, Artificial Sequence
  Probe  1449044_at
aatatcttaatgtgtctcgctggttttgtcacattcaacattacccagacatcaggcaacatctgtccagtattgtcttcatcaagaacagactctatgctaactcccactagaagccctcgctgtgcagcanccagaagtgtaatggaaaatgtatcttggaccatggtactaatgtaaattaatatggcatcattgtttctttgttaatgttggtagaatttttaaagtgtaagctcaagtctaaatgttttatttgtcctgtagttgaggcttcgtaatttttttaatgtaaaaatcagctgttatccaaacaaatcaggatcaaattacagatcttgtttttatgaacaaagttgattttggtaacagttgaaaatcagtggctcaactttaaaaaaaaagcccctattgatgtcacatctg

SEQ ID NO: 141 - DNA, 447 bp, Artificial Sequence
  Probe  1427997_at
gatgtaaattcccttctcaaatattttgttacttttgaagtcaaaatcttacctccagaagacaggaaagcaatacaatcaaaatgaagaaaatcacgaaacaacttcccctgtatgtgttcccaagctccgcgcccttgttttgcgtgtattaaattatacaagttcctgtgagttgagggccataacgtttgaaagctctttgacctcctcaggattgttcatataattttttaaaaatttatgtatgtgtagttcctgggtatatgttagctgtattgatcatgtggcccaaatgagtcaatattaaattccactttctagtttaagttggtttgttctttgtagtagacactgaaagcaagagtcntcctgactcctataagactggcattgtgttagggacagataatcagcagggaaacacagtgactatatttcccaagt

SEQ ID NO: 142 - DNA, 486 bp, Artificial Sequence
  Probe  1428244_at
tgagccccgggtcaagttgttcaatgtttttgttctgtagctccttcccactcctttttgatgacatttacaaaagaaggcaagctgcttggaaggtctggacatgagggaaagggcattgaggacaaggctaatggtggggagtgctagaggcttgggttcccctgtcccctggatgggaccccagagtctcttgtcacagggcccctaggttttccccagctcccaccctctcctccagccatggttacggcagaggcagaagaaaactttgagcccagttctcacaggaaaggaaaactctcatgggctttgcagagaaggttccacctacgctcatatattgtctttaccatgtgctaggatatcacatcaacaggacaagaaaaatgtaaaatacttgaatgagcttgtattataacattaatattattgagagtntctgcttcccaggctgaagtgattcattcattattctggtcctgc

SEQ ID NO: 143 - DNA, 426 bp, Artificial Sequence
  Probe  1428248_at
atgggcaagcatgaggagctcacaattaagaaactttggacttttaaagagacttnggacttttgaagtgttggaatttttgaagactttggggggcttttttggagttggactatattttatattaagatattaccatgagatttggggataagaaatggaaagttattgcacaaagtaacatgtttgtgtgctaagttgacatgggatgaattgtgctggtttaactgtcaacttgacaacctagagtcacccagagtcttagtgaggaattgtttagatcagatttggtctatcggtatgtctatgggagattnntcttgatanttcattgatgtgggaaaacacancccnactatgagtggcaccattccctaggcagaaaatcctaaactgtatgaatggaaaaatcaagctgagcagcaa

SEQ ID NO: 144 - DNA, 505 bp, Artificial Sequence
  Probe  1449348_at
aacggtgccatttacttcacggaagccaagagaagacgaaaaggatggacaagcatataaatttgtgtcccgatcagagatggaagcagatattaaggctgggaagtatttggagcatggggagtacgaaggaaacctgtatggaaccaagatcgactctattctcgaggttgtccagactgggcggacttgcattttggacgtcaacccacaagcactgaaagtgttaaggacttctgaattcatgccttatgtggtgtttattgctgctccagagctagagaccttgcgtgccatgcacaaggctgtggtggatgcgggaatcaccaccaagcttttgacggactctgacttgaagaaaacagtggatgaaagtgcccgaattcagagagcatacaaccactactttgatttgatnatcgtaaatgacaatntagacaaagcctttgagaaactacaaactgccattgagaaactgaggatggagccacagtgggtcccaatt

SEQ ID NO: 145 - DNA, 535 bp, Artificial Sequence
  Probe  1449354_at
aagtcaagaagctaccactctggttcataccactcctccaagagaaacagggagtccgagaggaagagtcctcacaggtggaagaaatctcacaaacagacaacgaagagtcatgagaggcacagttcaagaagaggaagagaagaggacagcagtccaggtccacaaagccagagccacagaacctgagcccagctaaggcagcaccacttggacccaagactgatatcaggcttggcaagggagttcattttacaaatgaacaaaacttcattacagaataaaaacctaacacagtatccttcagattccccaaaatattcaaaacgaaactgattacaaaggaaaggcatggtgactccatctaattcccaaccaagttacaaaaccttgtggctttaagaaacagaaacaggtattcagtatttctttgtctgttataatgcttataatgctgttataatgcttagtatcatgttattgatgttattttccatacagtttaagctggcttcccatgttgttttcctgtaat

SEQ ID NO: 146 - DNA, 382 bp, Artificial Sequence
  Probe  1428315_at
tgcagactaaacaggccgccatggagaaatctgaaaaggccaagcaacttcgagcgcttaggaaatatggaaagaaggtgcaaactgaggtcctgcagaagaggcagcgggagaaagcacacatgatgaatgccatcaagaaataccagaaaggcttctctgataagctggatttcctggagggtgaccagaagcctgtggaacgcagtgcaaaggcaggaggtaaaggccagcagatgagtaaagggcctaatgccaaacgaagatacaaaaatcagaagtttggttttggtggaaagaagaaaggctccaaatggaacacgaaagagagctacgacgatgtctccagcttccgagccaaggtggctcacggcaagggctc

SEQ ID NO: 147 - DNA, 376 bp, Artificial Sequence
  Probe  428389_s_at
gggctagggtatctgtttatctttcaaaatgataaatctgactcaagattttttatgtatgtataaataagtattttggtgtgctacacagctttttcaaattaccagtaatttgtcttgtttctaaggagtgggccagtactaacttaacttggtgttcagcaagggatgcagacggagagctcccttctgcaggactaagcagatgcgtgtctttcccacccgtgactgtaagaatgctctctgggaggacttccaccggggcagagcaggctgcccagccgaagcgggaagggcatgttgggcaagctgctggagcgactgatgtacagttgttatttgtctataaatggagctgtttatggcaatttaatac

SEQ ID NO: 148 - DNA, 535 bp, Artificial Sequence
  Probe  1428390_at
agaaactctcaaacctgcatggaaaactcattcttctcgtcacgcaagttacagcatcagaaaaatcgaaaaaaatgacctctcccggacagaaggcaaaattggtgtatgaagaagagtcttctgaggaggagtctgacgatgaggttccagagaaggattctgatgataattgggacgaagatgaagacaaagacagtgaaaaagatgagggcgttgatgaagacaatgaggaggaagatgaggacatggaagacaaagaagagaatgaggaggacagagaagtgtcaagtgaaaaagaattaaatggagattcggatttagatcctgaaaacgagagtgaagaagaatgaagacagatgcagccagccagtcacacgagaggcctggcttgccttgcccagtgctgcctatagcatcctcccagggaggcttgccctgtgccagaaaacaagaggccaccatatgcttccaagttgacaggcaattcccacacttccttcctgtcctgagtgccccagactttaactgtgta

SEQ ID NO: 149 - DNA, 484 bp, Artificial Sequence
  Probe  1428409_at
gggactagtccatgtacctgtgagtcaagaaatactaaagtaatgccccagttgagtttgtgggtttgggttttgatttgtttgggctttctctttgtttttaacattataaaaataatctttgaggtaaaactaattctattaactggaagatacccagctatcttcatcattttttcaggaaagatcatttttattgtggggtaaataggttaaaattattctgtatgctatttgaatttaggttctaaagtaaagaatccgtagaggaatctatgcaatatgtaatttgtcaagattaattttcatctggggaaagaagttgctaagtgtctccaaagcaggtcactcaataactgaaagtcctccaaaaagagaactagtgggaagcatggtgtgtggtggtggaaaagaaaaactccctcagtttttggagggaataactttaaagatacttaagtggctacgtttacttggtgcagtt

SEQ ID NO: 150 - DNA, 280 bp, Artificial Sequence
  Probe  1428447_at
atcattggcaatgtttatttacccccaatgcctttttatttaatgcacaaatttatttagatgtttctgtttgctaatcctgctcttnaatnnnnttgttactgctcattcccnttnnntnntnnnnnnnngnaantngnggacttncaattttctgcttttgacaattccnaacactatgagtgaaataagttaaataantnaactgcatcctntcagacaatgggattcctttcntggnccacagggagtgttaatgaattataagtcactttgtact

SEQ ID NO: 151 - DNA, 545 bp, Artificial Sequence
  Probe  1428529_at
gggagcccaaagcatctgagagaattgataaatgaaagaaacatgcttaaaacatgaccgttttaaaggtgaatgctgaccagtgagatgccagcctgcgtgtgaagatcctacagcagaagtgcatggccagtgacacaagaacttgcgagagatgccagtccgcctgtgaagatcctgcagcaggagtgcatagccagtgacacaagacgtgcgaatgaaaactagatgtgaccatccagaaaaagcatagacaaaaaaatgcagtcttagtactgggatgtttgagatggggtctatctacatgtctntagctgtccctatcaggacagcatctggtgtggcctgcagagttgattcctgtacttttttttttttnngtgggctgtatgtaaatggattgttttgacaatccattcgacagtgttntcttctgtgaaggtcataggtgagctgattgacagacacagctgtacagactgaatgcaatcctgactataaagctgcagttcatcattatgagctatttccttattctggcagtg

SEQ ID NO: 152 - DNA, 499 bp, Artificial Sequence
  Probe  1428651_at
gttgaccttgagctagtggcaattctcctgcctctgtctgtgagtgctgatgcttttgtgaataccagctgcctgctaggcagaaagagaatgttaactgaggttgttgcttggagttttacatttttaacttttggtacctaggtgctttgtaaaatactcagataaatacccactgagctatttggaatagtgaagagtatgcgtagttatttctgtttgtcattatattagtagtgctgctccaggcttttccttgagaaaacacttcctcttcatcgctcccgctcctgcagtgtgagtctccagagctatcgaaggctttcagaaccgaacaagtgtgcgtgcttttctgtaacttccacacagccattgcctagagtgaatgctcacagtgaagaaaggagtgttctccatgtccttgttctccgttcagcagtagtacagtaatgtagtacagtaatgcttacagttttgtttaattgcacttcctccatag

SEQ ID NO: 153 - DNA, 380 bp, Artificial Sequence
  Probe  1428694_at
accagctggattttccctgtgcatggtttgaagagtcagtcctacataattgctgacacaatgcaacctgcaactgcccggagaaagaacagttcactaaaatttgtngtatttatcatccaattctgttctgtaactggtaacactagtttgtctggctttagagaataggtgaatctctaaaacagtagaaacagctcagttgggcaagggccgttctagtagcatgcctgctccttggagttttccagattattttgtatagcctattccagattcttgtctaagattgctttgctctgtgcactcagaattggtgtgcctttctatttgtgtaaatgatatataatacattgctagttgtctaggatctattcagg

SEQ ID NO: 154 - DNA, 435 bp, Artificial Sequence
  Probe  1428777_at
gtattttatcatgacaactccagagagtgtgtatgtttctgagtagatgttgatgaggcaggacttaaaatttaaagagaaaaacttgaaactacctattttcattggttattcagcagtgcctaaaatgagcttctgaactattacaaatgcactttggattcatgtgtacagtgccatctggttctctgggtcaaacttgcttctcctctccctctttcttctcccgtaactaagtggcacacaggcatttcttagtgttcgtgtcggtcttcaaagtgcagagaacgtggtggagagaattttatggatcttttttttctgttaaaattgaatgtctgattatatttgcaacatgtaacttggattaatgtgttttaaagtaattttaattgtagtccagcagcacattgagcaagtgtagcttttctgaaa

SEQ ID NO: 155 - DNA, 262 bp, Artificial Sequence
  Probe  1449886_a_at
gaatatggctgcacagataccggaatctgaccagataaaacagtttaaggagttcctgggaacctacaataaacttacagaaacctgctttttggactgtgttaaagacttcacaacaagagaggtgaaacctgaagaggtgacctgttcagaacattgcttacagaaatatttaaaaatgacacagaggatatccgtgagatttcaggaatatcacattcagcagaatgaagccctggcagccaaagcaggcctcctgggc

SEQ ID NO: 156 - DNA, 219 bp, Artificial Sequence
  Probe  1428869_at
tggactctcgagtggcggacaattcctttgatgccaagcgaggtgcagctggagactggggggagcgagccaatcaggttctgaagttcaccaaaggcaagtccttccggcatgaaaaaacgaagaagaagcgaggcagctaccggggaggctccatctctgtccaggtcaattccgtcaaattcgacagcgagtgacacgtggtcatcctttggcaaa

SEQ ID NO: 157 - DNA, 213 bp, Artificial Sequence
  Probe  1428870_at
atcaaggacaggctggtaagtacctgcatggttgagtcactggtcaaggaaatggagatgacctatgaaccctggtgtgaagtagatacactggatctcctgggcacctggagcagcagcaggagacagcccaaggaggcaggagggctctaactaagcaggaccattttgtcttgaagtcggtacacaggagtaattatgccctgttcaagt

SEQ ID NO: 158 - DNA, 438 bp, Artificial Sequence
  Probe  1450010_at
atgtgctttccgtttgcaaggtgacacgcttggtgctgcctggcatggtagaaagatctaaaggggtgattctcaacatctcctcagccagtggcatgctcccagttccattgttgacaatctactctgcaaccaaggcctttgtagatttcttctctcagtgcctccatgaggagtataagagcaagggcatctttgtgcagagtgtcatgccataccttgtagctacaaaactggcaaaaatacagaagccgactttggataagccctctgcagagacatttgtgaagtctgcaattaaaacagtaggtttgcagacccgaaccactggatatgtgatccactctctcatgggctcaataaactcaatcatgcctcgttggatgtattttaaaataatcatgggtttcagcaagtctttgcggaatcgctacctga

SEQ ID NO: 159 - DNA, 366 bp, Artificial Sequence
  Probe  1450011_at
cacattatatttggtctggaagctactataaagtgcattttttattttagcattagagggaacagggctcgattaatttgttatgggcagattcagtgtagtttacatcgagttctgtgagcttttctaagtgtatgtcccagcttcgtatttatgtggcactggtcttctcaaaggtggctaagacataaaaagagaattgaaacatggagttctttctaacacgtaagtccacaaaatgttattcttggaccacacatcatgtctgctctgttcaaatgtatgtaaatactgaatgctatatagtgtgcccggaatgtagaggtaagtgggnccaaagtgtacatcaagaccaacaacatcctg

SEQ ID NO: 160 - DNA, 290 bp, Artificial Sequence
  Probe  1428970_at
gttatgctattgcttatctgttggagaacatcccaggttagcttccactctgtgcctacaatgttgagtttaaagatttggaataagaggaatcagtagaaaaattccttctattcttggaacaatagacgtataaagtattaatacaaatatcactgtgaccttcagctgacaggtttggttaagaccagaccatcttcaggcacactgggagtggctctgatggattgagaactgtaatgtggagacatgtgagatacgttttgctcacattgtcctgtctcaaatat

SEQ ID NO: 161 - DNA, 504 bp, Artificial Sequence
  Probe  1450087_a_at
agtcagtacaaccaagagtcccaagccagctgtcactccgaagccatctgcagcaaaggcagtgacaactcctaagcaacctgcaggcagtaaccagaaacctcagagcaggaaggctgacagcagctccagcgaggaggaaagcagctccagcgaggaggaggaggcctccaagaaaagtgccacaacccccaaggccaaggtgactgctaaagcagcacccgccaaacaggcccctcaggctgctggggacagcagctctgactcagatagttccagcagtgaagaggaggagaagactcctaagcccccagctaagaagaaggcagcaggtggagccgtttctacaccagcccctgggaagaaagcagaggccgagagcagcagcagcagcagcagcagctctgaagattccagtgaagaggagaaaaaaaagaagcccaaagctactacccctaaaatacaggcaagcaaggccaatggcactccagcttctctgaatgg

SEQ ID NO: 162 - DNA, 543 bp, Artificial Sequence
  Probe  1429061_at
atccctgtcatcacttggtattgtcagactttaatcgaagccattctaactggtatctagtgacacagcagtatggcattaatttccattattctaatatttaatgataaggaggctccttttgcccactttttaaatgggatcagttatcttatcaagaatcatgaggtatttttttgttttttaattctggattcaagttcttaaagaaatatgtgacttgtgagtattatctccatatttgtgagcgcatccttaggcattttcttaatcatctttgaatagacccagttttcccttcccaatctacatagttatttcttctttgtcttactgaagtaggtagcaacccaggtattgtctgatcactgaggacatttgaacaaatgttaacccattgattagttaacttcatccaacttcattgtgtagtttttgtattaaagtgctaactgccagaagcacgcagaataacctatcttagtttttatttattttatgtgcaactatatttgcctaatatgcagctcttattccaaaggg

SEQ ID NO: 163 - DNA, 538 bp, Artificial Sequence
  Probe  1429080_at
ccgcctgtacctgaaattaaagttgtatcgaatctgccagccataaccatggaggaagtggccccagtgagtgtcagtgatgcagccctcctggctccagaggaaatcaaggagaaaaataaagctggagatctaaaaacagctgctgagaaaacagctacagacaagaaacgagaacgaaggaaaaagaaatatcagaagcgtttgaaaataaaggagaaggagaagcggaaaaaattgcttgaaaagaacaacccagatcagtctaagtccagcagagcagcagcttcagagaagctcaaacagttgaccaaaacgggcaaggtgtccttgttaaaggatgaacgaaaagataagcccttaaagtcatctcaggcattcttttctaaattacaagatcaagtaaaaatgcaaattaatgatgcaaaacagcctgagaagataaagaagaaaaaacaggacatttctgttcataaattaaagctgtaatatatttaaatataatgtataaatctgtaagtttctgtcatcgttatgt

SEQ ID NO: 164 - DNA, 347 bp, Artificial Sequence
  Probe  1429268_at
aaaccagatcctatctttcgagatcagtcaacctccgcatgcctgccaccagctctcttaccaccaccgctgctgccgccaccaccacaacctgtcaccaccgtggcctctgtagtggctgcatcaccctctgtctacactatgccccgggtctttggccctttccctccactgtctggcaagccgatggagaaactggatatttcaagttctgaagtgaaactaaactggagtggtctttcatcaacgtcaaagagccacatggttgatctgacccccttggttctcaatcccggaggcttccatttctcatatttggcaggaagcagtgcacacaagctccattg

SEQ ID NO: 165 - DNA, 381 bp, Artificial Sequence
  Probe  1450387_s_at
aggctcctgtttaaaacctttgcagggagtaccgcaccttctctctgggatttgggaaaagggaagaaaaattgaaaaccccaggctctgtgtggactgcaagaaatggtagagctcaaggcttctctagtcttatgagaacttgtactactgaatttgcgaattttgtttttttgagctggaccgatctctgggggacgacgttgagctaggatggcatttcactgtgatcccccaagttcaagccagtctttaactcagtgttggcttaagtttccctggagctggcacgtcagcttttcttcagtgacagttggtgttcctcttgatttgggtatatgtgcttagttcagagtctgttctgtatctgctatcctaact

SEQ ID NO: 166 - DNA, 354 bp, Artificial Sequence
  Probe  1429351_at
gaatacagcaggaattagcacaactaattattgtaacttccagtgccctgggctgcagcaggtgacttgctgtgtaacttcccagtctctcactataatatactccttgtcaatgacaatggtgtcaatcgatttctgttcaaattcaggcagttatcagaaacctagcattggcaaacaactttttcctcatcttaggataatacaaatgtattttaagagtcatgcttagttggaatctaaattctgagaaaaatgctaaaaaatacacttaggaaatggttttcaagtcataaattataagtgatanagnctatagaaacaatgcagaaaatcatctgtcaggtgagcaac

SEQ ID NO: 167 - DNA, 540 bp, Artificial Sequence
  Probe  1429456_a_at
gcaagcagatactggtgccttttcccccacagactgctgcttcccctgatgagcagaaggtatttgccctctgggagtctggagacatgagtgaccagcatcggcaggttttgcttgaaatattttccaaaaattaccgagtacgtcggaacttgatccagtctcggctgactcaagaatgtggagaagagctaagcaaacaggaggtggataaagtgctaaaggactgttgtgtaagctgtggtggcatgtggtaccttaaggggacagtacaatcctgacaataggagcaaactgctaactcgacgaatccaagcccagggaaggatggcagagccacacgtagtnttgacttncctcaanacacaggaagaggatctncctacctggtggcctgtggcgctgcctggcccagaggacagaggggctcatgctcagccagtggcaggntcagcgtaagtgagagcgctcccagcagagaccagcgggccttctttgcagtacaatttgaaattcctgatgtattttgcttattgggtt

SEQ ID NO: 168 - DNA, 552 bp, Artificial Sequence
  Probe  1429612_at
gatgtgctatctgtgggcatttcctcntcctgtgagccttgcagcccttgtatggcggctttggtctgggcnagtgcatgtgttagtagtgtacgtgggcaggtcgggctttctaagctttgtcagataactatttttcctaagtatgtgtttcctactccagtgattgacaagtgtgcattgcctatgtttgtttatactttaattacaaaactattttttttaacttcctacattgttttcctacttttttgttgttggattatgtaacttttcctaagttcagaagtcttaagtgtgcaagcgtttatgtgaccgtnccattccagagtgcatcagaactgtcatcccttccaggaccttctcaggagtaacacatcaggtatgtcatcagcgctgggaaccgtgaacnccaaggacactcgccactattcacacgctgtnccgagggtgtgtggagggacaacagagagcagtgtatctgagtaagatgcacacatcgaagtgcgttcaaagtctgtgtctaggtatatatgtccgtgtagctctctc

SEQ ID NO: 169 - DNA, 272 bp, Artificial Sequence
  Probe  1450698_at
acagctctggctttgactgactcctgggggaagctgctgtggcccagctcctccctcctcatcttgccgtcccactggggtgctaggaaatccagngatgacggctgctctgatatggtgctcttctgaggtgggcataagggctggcccttatttgctctccctccttcgacttgcagaaattaatttaattaattaatttactatattaaagcccttggtcacccaagggctcagaaaacaagctgtgacaagcagaaaccatgtctagg

SEQ ID NO: 170 - DNA, 568 bp, Artificial Sequence
  Probe  1429689_at
tgaaggaccttttcatctctttctctttttttggtccttatatgaaataagtagatctcagaacatacaccatcactattctaagatcaggtattttatgaatttatcccgaggtgacagaagagatggcagagtataatccctctgacagcccaatctgggaaacatgtggccaaggtttctatggtgatctctccctcccagacacattgacactgtagactgcattcattacagcgggaatattttgaagtgggttggatgtatatttttttttttacatttttattgtttagtttgtgcccagaaaaatggttatttcatcatgtaacattcctatttttctggaaagacttttctgttttgatgttcttgaattattttctgacctggaattatcctttcaaaaaaaattcttaaaagatgtagagctaaaaagtacctaatgagaagctgaagctggcccattggttnaaagtattgattctatcttgttatttaaatgtgaacatttattgtatattcagtgagttatagtgttaatagtcttgtgctacacagcaggtgt

SEQ ID NO: 171 - DNA, 388 bp, Artificial Sequence
  Probe  1450873_at
gtttgatatgaagcccaagcacttgctgtctgggaagaggaaagctggtaaaaaggacaggagataatgtcctcttcagttggaacagtttaagtagactcttggttattttgggtgntggtatggtgtgttatagaatttggaatcancatctgttnaccaaaagaaacagtgggaatgacttttaaatgcttgctgctttgctggaaactggcaaaccctactaagtgtgcagtgctgtgctcactgcgcagtttgggtttttatgtccagtgcgttccntacttgatgagcttctttcagcattttacagaaaagacttattggtgtacggaggctgcgtgggtggaggaaaactgttttgtagtgggacttttgtcacctaaag

SEQ ID NO: 172 - DNA, 451 bp, Artificial Sequence
  Probe  1450914_at
ggaaggtcaccgtcaagtacgaccgcaaggagctacggaagcgcctcaacctggaggagtggattttagaacagctcacgcgactctacgactgccaggaggaggagatcccggagctcgagatagatgtggatgaacttttagacatggagagtgatgatacccgggctgctagggtcaaggagctgttagttgactgttacaaacccactgaggccttcatctctggcctgctggacaagatccggggcatgcagaagctgagcacaccccagaagaaatgagggtcctgactcagacggccgtggctcccacaggacaatcgctgccccccaacctcgtagcaacagcaataccgggggaccctgcagccaggcctggtgccatgagcagggatccccgtgcccctgcctaggggtcatttcccttgccctcagttttccacttttgg

SEQ ID NO: 173 - DNA, 552 bp, Artificial Sequence
  Probe  1429897_a_at
gaatatgttcacctctgtcaagatcgctgcattttggcaatacggtgtttttataacttatctttctttccaaagatacatgtgtttcggatagtattcggagaagcagtctacctagctgagctgttttaactacttaacagaatatgaaagtggaattaatngttcctttaaagcaaatatccaagtaaacaaagcagcagattattgttttctaatggaaacagtgctgaattcgcttaggaagaaaagactattctagtttgtatgtgtcagtctccataaaagtagtggccctgccctgcttcttaattctcaacactttcttggatgtnttagttttggacaaagtagntcaactntnttcagactgcactgntcttcctttgggggaaaaaataaaattattatgtgtgccacttcacatggagttcccgtaatntacgaactgtccaaattntgctatccacatcactgaaattagtccaaaaaataataataacaaaccattaaagaaagaaaaggaaaagcatttcaaggtacttttctaag

SEQ ID NO: 174 - DNA, 463 bp, Artificial Sequence
  Probe  1450986_at
aatgatggccattgcacctaacgttacagtcatggttggagaattagttggagcacggcttattgctcatgcaggttctcttttgaatttggccaagcatgcagcttctacagttcagattcttggagcagaaaaagcacttttcagggccctcaaatctagacgagacacacctaaatatggacttatttatcatgcttctcttgtaggccagtcaagtcccaaacacaaaggaaagatttctcgaatgctggcagccaaaactgttctggctatcagatacgacgcttttggtgaagattccagttctgcaatgggaattgagaacagagccaaattagaggccaggttgaggattttggaggacagagggataagaaaaataagtggaacaggaaaggcattagcaaaagcagaaaagtatgaacacaaaagtgaagtgaagacttacgatccctctggt

SEQ ID NO: 175 - DNA, 568 bp, Artificial Sequence
  Probe  1451016_at
ggaccttgaggaggacttcgtctatgaagacatggaggccctctgtggatctctgcgtaccctagccaccgacagcaataagtaccgtgccaaggttgaccggcggcgccagcgctccattttccgagctgtgctgcactttgttgagggtggtgaatgtgaggaggagacagtccgcttcggactggaagtgctctacatagacagctgggctcgccaccgcatctacacagccttcaaagatgtgttgggctctggcatacactatcacctccagnnnnnnnnnnnnnnnnnnnaacaatgagcttctccgtgacatctttnggcctgggccccgtgctagtgttggatgctgctgccctgaaggcctgcaagatttcacgctttgaaaagcacctgtacaatgctgcanccttcaaagcccggaccaaggcccgtantcgtgcaagggacaagcgggcagatatcttgtgaatcagactgnccaacgagaaggctacccataccataccatatttttaacagaagacagtgcaagaactggtccctgccagtgattgtcacttta

SEQ ID NO: 176 - DNA, 557 bp, Artificial Sequence
  Probe  1451115_at
cctttaccagatcttcagcctcttggggaaaggcactttcctgcctcctctcccaagactctccgttccctacccaaacccaaatatctggtctttgacatccattccgttctctttgaccatacagtcgggcgacaggtcagagatggacagagaactctattttggattgcagatttcttttttgtacataaataagaaaactccaaagcatccaaagatgaaatcccttgcctttcactccccagtgtgactcgaggaggaaacaaggccttagttgtgattcccaggggctggggagcaaggcgtggcctntctgctgcctgggtctctatttatatatttaagttcacaatgtttttatgctaaacagccaaggggcgaagcgntctagtgncctgtgttcaggcctggattactcttgcaccttttgtgggacatgggggctctgtgttntcctccccaatactcagnccccttccttcaggnccaaggcccntgtgatgtgattttacttttattcccaaagttgtagcagagttctcgcctacaataaa

SEQ ID NO: 177 - DNA, 159 bp, Artificial Sequence
  Probe  1430127_a_at
ccatgaattacctggaccgtttcttggctggagtcccgactcctaagacccatcttcagntcctgggtgcagtgtgcatgttcctagcttccaagctgaaagagaccatcccgctgactgcggaaaagctgtgcatttacaccgacaactctgtgaagc

SEQ ID NO: 178 - DNA, 509 bp, Artificial Sequence
  Probe  1451164_a_at
gagccccgggatgctgactttggtactgtgcatggagctgtcagtgtgactccaccagcccccacactgttgtcaggtgaaccttggtacccatggtacagctggcaacagccaccagagagagagctgtctcgccttcgccgactctatcaaggaaatctcctagaagaaagtggcccgccacctgagtcgatgccagagatgcccactacaccaccagcagaatcctccattgagcagccaggctcccagagtgcttagaacttgtngacctggggaagtcagaccaagggagtactgtctagggctacgtggtatcctgtntcctgcaaagggttctgttttgtgacagtggcagaccctatagggaactggctggcatgaagagaacaaagcacatttgacgttgagggggactagatgtttatgggtaactggtgaggacttacatataaactggagantcttgtgtatgcccttgatggtgtgtccctgatttaaacctcctg

SEQ ID NO: 179 - DNA, 304 bp, Artificial Sequence
  Probe  1451167_at
ctctggatcgggaaacctggtgacaagcccccacccctctgtggagccattccagcctcaggggactatgtggccaaacctggagacaaggtggctgctagggtgaaggctgtggaaggggatgaacagtggatcctagctgaagtagtcagttacagccatgctaccaacaagtatgaggtagacgacattgatgaagaaggcaaagagagacacaccctgagccgtcggcgcatcatcccactgccccagtggaaagctaaccctgagacnaganccctgaggccttgttccagaaggagca

SEQ ID NO: 180 - DNA, 544 bp, Artificial Sequence
  Probe  1430185_at
tccagagcagcagttacatgactaaggaagctgatgtgggggtcttggtgacttcagtttttagactgagtactgtttcaggctggcttcnntnttaacactgaaatgaggtctctaatttttttaaaaaggcacaacaacaagtctattttggggaccttggcaggttggcatttcagaagcatgggagtgactgtaagctgatgcctggctgtttttcctttctttgggtgctctgtccttggccagcctggatgggattatttntccttcttgttctgtgatcacaatttgatcctagtaactgggaactatgaagtgtgtgtctgtagcactggatctgaaaaggttnaaaaaaaannnaaggagggagggggagagagagaaggaaactggcagantcttagacttctgttcttaagtgtatatataactgtggtgaagatactaagaaatggtacaaagtttttaaggcttgaagaaggaagttttaaaagttaatgtgattttgctgtgatatattttgtgtcggtgagttacttgt

SEQ ID NO: 181 - DNA, 252 bp, Artificial Sequence
  Probe  1451254_at
ggcagatcatgaactggagtctcgtttctgttacaaccttggacagctcatcattgatttgatcctcagttcctgttgttcatagggaatgatgtttcatggtttctacctcaaaaagtgaagacaagtggaaccacgtttctaagttgctctgtgttgttaacatagtgcatggtattcatgtaggcagttagtgctgccagcttttagtaagatgtgattgcatagctggcatctgtgcattaactcaaa

SEQ ID NO: 182 - DNA, 214 bp, Artificial Sequence
  Probe  1451262_a_at
aagggaacatcgcacgtttcctgttctctctgtttggccagaagcataatgctgtcaccctcaccctcatcgatagctgggtggatatcgccatgtttcagcttcgagaaggcagcagtaaagaaaaagcggccgtgttccgctctatgaactccgctttgggngnnggngncccgtggctggttggaaatgagctcactgtggcagatgtggt

SEQ ID NO: 183 - DNA, 359 bp, Artificial Sequence
  Probe  1451293_at
gaccgtgcgtgtgtggaagatccccgaggagtcccagcttgtcttctatggccaccagggttccattgactgtatccatctcatcaacgaggagcacatggtgtcgggcgcngatgatggctctgtggccttgtggggcctctccaagaaacggccactcgccctccagcgcgaagctcatgggcttcacggggagccaggcctggagcaacccttctgggtgtcatccgtagccgccctgctcaatacagacctggtggccacaggctcccataatgcccgtgtgcgactttggcagtgtggagagggcttccggcagcttgaccctctctgtgacatccccctggttggttttatca

SEQ ID NO: 184 - DNA, 540 bp, Artificial Sequence
  Probe  1451385_at
gaaagaaatgcttcctcgtctctaaggtttaccagaaacactgacttgaagaggatcaatggattctgcaccaagccacaggaaagtcccaaaactccaacccagtcttacagacacggggtgccgctgcacaagcccacagattttgagaagaagatcctgctgtggtcaggccgcttcaagaaggaggaagagatcccagagacaatctcatttgagatgcttgatgctgcgaagaacaagctccgggtgaaggtcagctatctaatgattgccctgaccgtggcaggatgcatctatatggttattgagggcaagaaggctgcgaaaagacatgagtctttaacaagcttgaacctggaaaggaaagcccgtctgagagaggaggcagctatgaaggccaaaacagactagaagtatttggaaaacccaggagtaaggctgcaacaagaaacctgctttacaaaggatacaatctgagactcactttgcagacatgtattttgcacagatgtattccacttccctattaatgctgta

SEQ ID NO: 185 - DNA, 547 bp, Artificial Sequence
  Probe  1451445_at
acccggtctacacatctataagattagcaactgcatacactcagcaagtaatggtgcatcctgtagtaaagtggaatcaaagtagcaatagcacgtgcttgaaggnagtccgactgcagcatggtcccatcgagtaaaagcataacaaggcatgagcctcaggtgggttttgagctaatgtctgagaaaangctgtcaggnagctaatggaacttcccttctaggnctttagcttagtcatagtctccaacangctatgtnggttccanggagtccaagnnccanggagttgtaagatgcagacctaaactgagaccctgaagttnaagtncacacncacgctctttagagcangnttttaggnaaccnaaaaggnnaaaatgcattaagttctacanctgttnaactcangatcangtattnnggnnataggaaaccaaagtcctcttgaggacgacttactattccacccattaaaacttaacaaacagtacccttggccaagatcagttcatcaaaacatcatgttcacatgctgtctgctttt

SEQ ID NO: 186 - DNA, 373 bp, Artificial Sequence
  Probe  1451459_at
aggtttagacttcccatgctgtgaaagagcttgcgacttcgtcacaggagcctctctagtattcaggttttaaactggtaactgccacagaacctcaggcacaggttacagagaatggaagagtgtgagcagatgtcggaaaagaggaaaggaaccctcttttctgcagcgtgtgtaccgttggtaatggcgcctgatggaagccatgatgttcatcaataaataagtgttgtcaaataatttatgacaggtaattgatagtgtgtaatgaatggacgcttaataattgatacctaaaagcagactttgtcagctaagctttggatgttttcgtgagcagtgtgctttgtgtgttatttttctaccgagtagc

SEQ ID NO: 187 - DNA, 386 bp, Artificial Sequence
  Probe  1451466_at
ctttggtttcatattcttagctactaagttaatgatgacacaaaagctagtgcaaaattaagatgaactctttagctgatgcttagaatgtatttgtgtggctgataggttttgcctgcagaaatctaaggaaagaaaacccattcattcacttggcagcccctcacttggcagcccctcatgcataaagatggtttctgccttgaagtcggcagtctgtacctgggcatgggtcagactttaatattgaacataaataatatttgactaagcaaacagctgcctcactgtagaaaaagcaccttttcctgatgctaggatgtgttaaactggcctggctgtgtgcattagtcaatgtcccattggttttgctctgtcctgaagaa

SEQ ID NO: 188 - DNA, 383 bp, Artificial Sequence
  Probe  1451509_at
aagaaatcgtgcatcagctgcccagtaatgaaccagaacagctcgaggacaatatcaaccaaatctcaaaatggattgagcagtgggtcaaagatcataacccttaacttagacactggctagccaggcaccacctccgtccatcgtcccagatcttaggaggcagcagctggtctacacagtgagttctaggccagccagggctgcacagtaaggctctgtttcaaagggaaaaaagaaaaagaaaaaaagcctggctaccgtcactcagtccacatagtaggaacacgggaggatgagcagaagaaagcttgtngtcgcagctctagctgctgcctggatttctttttctccgtgagaaaccttacacaatctcagatatg

SEQ ID NO: 189 - DNA, 362 bp, Artificial Sequence
  Probe  1451884_a_at
ctcttctactcctttttcaagtctcttgtgggcaaggatgtagtcgtggaactcaagaatgacctgagnannnnnnganncnnccnnnnngnnnnnnnnnnnnnnnntnnnnnncnannngtcggtctagttttgatttttccctccacgctgatttcagcataaaccaatgcttcttccctctttgcccttgacagcatctgtggaaccctccactctgtggaccagnacctcaatatcaaactaaccgacatcagtgtcacagaccctgagaaataccctcacatgttatcagtcaagaactgcttcatcaggggctcagtcgtccgctatgtgcagctgccagcagatgaggtggacac

SEQ ID NO: 190 - DNA, 188 bp, Artificial Sequence
  Probe  1430896_s_at
ggaccagatattctcacctgtcctctaataaattctctgtccttgtaccactgctggccagaggnggaaagttgtatttgatgttcacggtccgctcagacaagctgaaaagggaacctggagaagtctgcttccctggaggaaagagggacccggtggacacagatgacacagccactgctctccgt

SEQ ID NO: 191 - DNA, 426 bp, Artificial Sequence
  Probe  1451956_a_at
atcaccctgattctgactattatcgcagtgctgatccaggccgcctggttgtggctgggcactcaaaacttcgtcttctctagagaagaaatagcgcagcttgctcgacagtatgcggggctggaccatgagcttgccttctctcggctgatcgtggagctgcggaggctgcacccaggccacgtgctgccggatgaggagctgcagtgggtatttgtgaacgcgggcggctggatgggcgccatgtgtattctgcacgcctcgctgtctgagtacgtgctgctcttcggcaccgccctgggctcccatggccattcgggagagacagttgtacacgggcctggagaagcaacggctctggagtggggaccaaacacgtggatggtggagtacggccggggtgttattccgtctaccctgttcttt

SEQ ID NO: 192 - DNA, 382 bp, Artificial Sequence
  Probe  1452012_a_at
aattacctgctgactactgctgaaaacgagctgggcgttgtggtggcccacagtgagtcaggtgttcagatggttcccatcagctggtgtgagatgcagtgccccaagacccacactaaagaattccgaaaagtggcccgagtacagcccgagttcctacagacctaagcccacctaccccaaagaggacttcagcacttcagctacttctaaaagtacacatataaaaaaaccatcaagatgcaatggtctttattaaggtacctgcggtcagctggcaaccatntcttgaaacaatgtgccggaaatgtattcatgctgtaatgacacccaaggcccctnccgctgctgagacagggagattgttagtgcccaagtttga

SEQ ID NO: 193 - DNA, 536 bp, Artificial Sequence
  Probe  1452047_at
agaaaagccttcctacgacacggaggcagaccctagtgagggattaatgaatgttctaaagaaaatttatgaagacggagacgatgatatgaagcgaaccattaataaagcgtgggtggaatccagagagaagcaagccagagaagacacggaattttgaggcttttaaaagtccgtatgggaactgtgatgtggaatgctcgtgttccagtaaggggatgttgttgaactgcacatatttgttcatgtgggtatgtagttttgacagataaatatttacaccgccttttaagtaaagatagcagattctccatttcttactggaggatttgatttnaaataaataagcttattaaactgtttccttcattagcaccccagacatttcgtttnncagaaaaggcntatatggcactcttactgaaaatgtaaaaaggaantcttgcataatgaaaatgttatgtcacagacttttgttctggctgggagatagaatacagaagtgtttgtctgtagttgacattagatcccagtaa

SEQ ID NO: 194 - DNA, 350 bp, Artificial Sequence
  Probe  1452052_s_at
agaagactgcttatctcagagtgaagataactgtggctgaaaagtactagtttgatacttaagattttaatgaccaaaaccctccgacttggaagctgaagaaggtaaacttctccattattacagtgcaagtggtggactctccagtgcattgcctgtctagttatttatcagcctgtttctattgatggaggacttcatatgngggagggaacnctgtttctgtttgcctgattcaagtgtctgagaaacaaatgttctaggctgcagtgggaaaactttgaacagggttttggcgtttccttttctttataaaacgtgctcagcaaactgcaccagttacctacagt

SEQ ID NO: 195 - DNA, 529 bp, Artificial Sequence
  Probe  1452094_at
agatttcaccacaagtcaggagctggtggagcaggcgaggctgtgtggcaggcgtgcaggtggcaggcgagcagagcctgtgggcttacagacggcaggcatgcagacgccagtgtgctgctcttccgatgtgtggactgctgagcactggtaactaactagggcccagtcaggtctgctattccacgtgcagagttcctattatttaaagaaactctttttatcaaatccccctcctcaccacgggtgaattcagccagtttgagaaaattactttgttcaaaaagtcctgcagaactggcctcccaaccgagtgctcanccttcncccttttcctcagtgactncaggttgtccagagactnttccaagtnccaatcagctgcancaggcactggtgttggcattctgattcaattctgtnccttaggagactgggagttagaaaacggacaggtccttggactgagggagttgatttttaaaaagaaatgttttataagaagccaagatgtttgtttttctccatt

SEQ ID NO: 196 - DNA, 523 bp, Artificial Sequence
  Probe  1452099_at
gctcgacacttctttctgcaaggagagaagcagtttctgctcctcacagaacgcttccatttctacaaaaggtacacaataaaaggcatcaggaacctgatattctatgaactgccaacgtaccctcacttctacagtgaggtctgtaacatgctgagagccaccagcagaggagaggaggccacgtggacgtgcaccgtgctgtattccaagtatgatgcccagaggctggcggctgtggttggtgtggagcgggcggcacagatgctgcagtctccaaagaatgtccacctcttcgtcaccggagaaaaatgagtgtggaggggnaacggactacattgttggacctcatagacctgatctgttcactnccccaggaacgggctccaggcantctgcantcctgtgtcactggaaagtgcagtgtgcagtgcagagtgaccagtgtgggtcaccacagctntcagggccaagagcagtcagagncctgctactctcagaaactgacgcttgtgcttgccac

SEQ ID NO: 197 - DNA, 392 bp, Artificial Sequence
  Probe  1452119_at
gaagtttgacactcggttcttaccaaagcccctgttcttcaggaaagccaagaacagctctgccacccgtccccaaggtcctgctggccagctgaataaaacaccctccagttccaagaaagtcacctttggattgaacagaaacatgactgcagaatttaagaagacagacaagagtatcctggtcagccccacaggcctctccagagtggcctttaaccctgagcagaggccactccacggagtgctgaagaccgccacgagctccccggccagcactcctctgtcgcccatgaggctaccagccaccaccccaaagagaaggccaagggctgcggacttcttctgagagccagaagccctttctgaagcctgcttttataccggatgtt

SEQ ID NO: 198 - DNA, 273 bp, Artificial Sequence
  Probe  1452171_at
tcttccgcaccatcagtgtttgaggtggccaacacttggcctcaagacttggctccacatgggaactggatcggttaacttggcaaggatcattaggcctgtagactgcacctcaggaaggaagacttgttcaactggcctacgtggaacagaaggaacggactctactgacctggtatctctaaggatttggattgacccttggccctgccttacaccaaagactaggtgcaggcaaagatccttgcctgtcccagatacggcctcaggatg

SEQ ID NO: 199 - DNA, 347 bp, Artificial Sequence
  Probe  1452172_at
aaatctgtgcacataccgttgccacagctgccactgtcagcatcacagccaaacgagaagcttgcagaggtcctcagcaggcttctggaaggggaaggacgcttctcgagaaatgtgccattgccacataattatcacattgattttgaaatcagaatggatactaacaggacccaagtattttcattttctgatgtagatgcaagttctgccacaaacatgcagagagtagcagtgctgtgtgttcctaagtctgtttactgtttaaattcatnccaccccagaggattgatggccatgaaaatccgacatttgaatgtaatgggcttccatgtgatcttgatcca

SEQ ID NO: 200 - DNA, 359 bp, Artificial Sequence
  Probe  1452203_at
atggaacatcacttacagtgggtgattggtaatttttttcattgttagtatttgtctctccttaaatattgtctcttgagtaaaacttgctgtaatgatgacatatctacctacttatttttactttcagtaaacatggagagcccagtccattttactgaagttgtattcatcttcagcttgactgctccctaactctgtacctgtcatttctccttacctcatattgtaaaataagtaagtttctgttcaacttgtatagcatttgaatataatttgtgtcagattttgatctaattcattttaacagattttcagtaagcactgcattaggctctgtgtggagattatcatagcat

SEQ ID NO: 201 - DNA, 193 bp, Artificial Sequence
  Probe  1431182_at
gaaccaggtatgtgttttgccaataagtagatgttcattcagaaaagggctgagaggggtcacaatgatgatgaatggtccaaacattcgcggtttccaccagaacgcaaggcagtgttggcagttaccttccttggatgtctgagtgactcaatagctaagggttctttaggcttagttgtcagtctcctcc

SEQ ID NO: 202 - DNA, 439 bp, Artificial Sequence
  Probe  1431302_a_at
aaagaccctgagactggtgtgaactacctaatccagggaatgacctcaaaactggctgtgttggtggccttaattattttggaacaaagtcctgccttcaagattgattttgatctccatgacctgataccgtcttgtgaaaggaccttcctttggagatattctttaagcaagttgtgaagaccctgaaacccaaaagaacgtacatgaggtttttgtgtgagcttatgctcagaacaacagtgtttagcaagtggcatttgacaggtgtgaatattttcctacaatgtggctgtgagatccttgcctgacttcctgttgctgctttagaacactaaaagtctttcaaaactggaaaaatgccctggcaggggagaataattttacccatttaaaaaaatggcccgatttttagtcatattagggatatctgtcacat

SEQ ID NO: 203 - DNA, 72 bp, Artificial Sequence
  Probe  1452439_s_at
atgaatcggcaagcggcttaacagaggacattgggggaaaggaccacaaactcagtccatcggtccatggaa

SEQ ID NO: 204 - DNA, 519 bp, Artificial Sequence
  Probe  1452753_at
gaccctgcagccaagtatcaagcattcaggccattttatgattctcaagaggtttctctggattttcagaatgacgtcccacactaatcctgacttatgtcacatagcaagtaaactaagagggaagggtgaggagaagtggggaggccagcagcccagcccacactcaggatcaccgctcacctttccagagcattctggactgagctgagcaacaggcagagggcgttagcctcattgtgaggtttttttttnccctgatgtcatcatcataacgttatttatttaaatgtagttattttggtatttaattttttttnagagagngggggngaaaccctgtattttcctggtagaatgaaatggctcagaatggtatatttagncaatttaaaacattatttacataaagaccaaatatgataantctnttntaagtattgcttcacccacagtatggagctgtcacaatgttactgcagatgatgcgtgttgacatggagttactgcacccaagct

SEQ ID NO: 205 - DNA, 326 bp, Artificial Sequence
  Probe  1452902_at
gaaggatgaacctacaccctaactttcctaacccccgggatagaatgcttttgtagcccagtgggatccctcaaaaatctcagttgtgtgcctgggcactgaagggttgccacatttacctcggaacctcttgtcatgtcctgaacagcccttttccttgtgaaaggaaatgaatactacttcctaagagacactgtaaccccagattcagggatgatatatgagatactgtgttgctctggaatttgattttcagatttctgagcccnttccctgagtagtgatctgcccagggnaaagggccggagactttgtggttgatgcac

SEQ ID NO: 206 - DNA, 474 bp, Artificial Sequence
  Probe  1432164_a_at
gaaactgttcttgacagtgctacttaagagccacccccaacttcctaaaggatgcagataaacacagtgtacaggtttttgataattgccatactaagtatttagactaggggttaagccctggtgtttagaattctgaggatcgtctgctgtgaaacgtggttatgaggctggttttggtgacggaccaccttctaatcccagcacgcaacaggcagatgttagttagttcaaggtcggtctacacaacaagttctagaccagccacagctacatggcgagaccctacctactgggtataagactttcataattcaagataatattggtgtatcttagataattttgttacttgcccatatccaccactggatttgggtcagagattagtgtcttctcattgggaatgcaaggaagtgttgtacagtgtcagaggcaggagagtactgtctttgtttcaccatgtgctgtgtt

SEQ ID NO: 207 - DNA, 389 bp, Artificial Sequence
  Probe  1453195_at
tctaggacgaggtcgggctcagcctcagtgttgtcaagtggaacacgaagtgagccagtgtgtaagtgagcagcctgtacagagacgcgtgcctcactgaggcagcaccgcttggtacgtgctgccgtcctcccaccaggaccataggaccaaggtcacacactgtgcacagtgcaaagcttcatacaccctctactgtgtgctgtcagctttggaaagccatttggggctgtcgattttgatncacnntnnatgtgtttgtgtnccttttgtatagtnncaattcagactggaattaaagcacacacccacctggggggtcagcaccagctntttgggcnggctggctggtttttgaaggacaggcaagacataagcagctccctgtg

SEQ ID NO: 208 - DNA, 296 bp, Artificial Sequence
  Probe  1453745_at
gccagtcctgctagttcaataattgagctgttctccgaaacagtatcctagggtagaaacatagatttagcggtgaagatgaccagaccaagtctgcagccaccaaaacaagattactagttctcagaaaaaatttcctgcccttccctgcactgaattccaaagaaaaccacaaagtgccctgatcttgccaccaaaattccctgagatactaattagctgaccaatgaatttacaaatatattgctgctttgctgaccaatcacaatatgactattcaaccctccataaaaaga

SEQ ID NO: 209 - DNA, 556 bp, Artificial Sequence
  Probe  1453983_a_at
gaggacgtcatcttagccttggaagagagaaagtccactcccaaagagctgagtagtggccaagacgtggcccacagcccccaagagagtgccctgtgtgggcttgatgtgccgggcggcgaggctgctgctgatggaggccattgcctcagccagaagttactgtgccaggaggagactcccgaggccacggaggatgaaagggatgaggaaaggggagggatggaggccatggaaagttgtaaaggctccagcaatggagcccaggacggagaggcttctgagaagggggatcgcctggatggggcggccggacgctacctgtttaagtgtttagtcaacattaagaaggaagctggcgatgccgtggttgaaatgcattgggttgagggccagaatagggatttgatgaaccagctttgcacttacgtacgtaaccaaattttgaggcttgttgctagttaattcttaatttcttcctcagttagaaaagtgatgtatggggctagggatatgatgcaatgaccttatgtgcttagtatccagaagttaccct

SEQ ID NO: 210 - DNA, 374 bp, Artificial Sequence
  Probe  1454214_a_at
caacagtttttgaccataggaagcaacagttttgttggcatgtcaagctccagtttcctcagagtcaagcagagtacatagaaaaaagagtaccagatgataaaactattaatgaaattctaaagccttacatcgatcctgaagagtctgaccctgtgatccgtcagagattgaaagcttacgcacagtcacagactggggtccaaattttaatgagggtcgaaaatatgcagcaaaatatgataaggtatcatgaactggatccttacaaaagcctttcagacaacttgaaggacaaagtgatcattgagtacccgactttgcacgtggtgttgagaggatctagtaatgacaagcagctacttcaagtgaaa

SEQ ID NO: 211 - DNA, 471 bp, Artificial Sequence
  Probe  1433467_at
agctttgtttacatgcttgggtcatgggacagtatacccgcttgccttctcttgtccaagggctgggcaggtgagattgaagtctcagaaaatgcttggtacttgtgcatctgtcttctccctatgaagatcgatgaagactgtgggagaaaacagcttccaccccagcctcgctcctggtaaacggtagaaactaattctggtgatcggactaggctgccagttaaagccaaagcaataggcgtgaatctctgtgagcagtgtgtctacacaagattgtagaactcttttgatgcccttagacattnnccaccttggacacagaagaacctgctatgctntcttggcatccttcaaggacatttcaagttttagaatttgtgaacaatacagatagtaactatgtggcagtctcccagtaccaaactggctctagactcacaggagtaaccttagatatatgcttgctca

SEQ ID NO: 212 - DNA, 173 bp, Artificial Sequence
  Probe  1433502_s_at
ctgtttggaaatgctgagacagcactgcagccagtgccacgccagcagtcggctcaatgagcagtttcattctcccccacaccagctgggttgcatactgttggcacagaaaggagcaagggttaatgaaacaagaaacggagccggagtctagtaggttacattcgaaaaat

SEQ ID NO: 213 - DNA, 156 bp, Artificial Sequence
  Probe  1433530_at
agagaatgcgcaggtacgtttaatttttcaagactaccttggggcagtgggggcaagctcggtgtgggatatttggttggaagaaaatatctggcgggaaggaagcaggagcnccgcccagtacagcagagctggtgcttgttaatctcatcgtct

SEQ ID NO: 214 - DNA, 303 bp, Artificial Sequence
  Probe  1433576_at
agagtactccacactgcacaatagctcctcccagggttttaactttgttttnnattnttcaaaaccaggtccaatgagctttctgaacagctggtgtagctacagagaaaccagcttccttcagagagcagtgcttttggcggggaggaggaaatcccttcatacttgaacattttctaattgcttatttattgtattctggggtatggcgtaagtacagagaagccatcacctcagatggcagcttttaaaagatttttttttttcttcaacaccatgaattcctttaacatgtttccagca

SEQ ID NO: 215 - DNA, 427 bp, Artificial Sequence
  Probe  1433593_at
actgggtctgtgcttgggtcattatttcacatctttccttagaagttcagctattgcatttgagctggacctgatagcacaccctattggaaagaggccagatttgacttccgacattgatttgacattacaaagtgttagaatatgcttctttatccaagctatttctaggcagactgatacttgcttttggtgaattaggcagggcagtgaagtcagctagtcatacaaataaacagatgggtattgtaggctcagtttccaagtgaaggaaatngcctgagnttgaacaagtgaaactttntntcattgactttnaaantggtttctgattagtaaagcttatgcttagctttncactcccangttaatggcatggnttntcttgagcagagatagtataatactgcatttccagtgggtgaac

SEQ ID NO: 216 - DNA, 541 bp, Artificial Sequence
  Probe  1454639_x_at
gagaatgcgcaggtacgtttaatttttcaagactaccttggggcagtgggggcaagctcggtgtgggatatttggttggaagaaaatatctggcgggaaggaagcaggagtcnccgcccagtacagcagagctggtgcttgttaatctcatcgtctnttnnnctcnnnnnnnnnnnnnnnntngcgccatcttccttgagactcctgcgccatgagagcgaagtggcggaagaagagaatgcgcaggctgaagcgcaagagaagaaagatgaggcagaggtccaagtaagccagcccgtgcacctacgacgcctgcaggagcagaagtgagggatgctgagggccgggacaagctatcggactgtgtgctgccatcggtaatgagtctcagtagacctggaacgtcacctcgccgcgatcgcctggagaaatgaccgcctttcttacaaccaaaacagtccctctgccctggacccccggcactctggactagctctgttctcttgtggccaagtgtagctcgtgtacaataaaccctctt

SEQ ID NO: 217 - DNA, 435 bp, Artificial Sequence
  Probe  1454659_at
cagcctcttagccttggtttgttggtcacatagcactgttaaatttatccgaggcataagtagttcctagaggagacagnctatcactccccctagaccaggcgctttgcggtgacagnctggagctatantggatgggtgatgagagacgttcccccatcctgtgtctcctgctgtgtggacacatgtcactcctgctccacatgccaacatggttcatttccaagggcaccctataaccagagagcacattaattcctgtcctataagagggctgtgggaaactgtgtacctggaggtggtccannncnnannncnnnnttnnnnccttcagcttcataggcagcatacncctgctnccccnattgtgggtgggggtnccccactgcgtactgtgtttttaatttcatttaacttctagcagacacgctcagt

SEQ ID NO: 218 - DNA, 347 bp, Artificial Sequence
  Probe  1433656_a_at
tcaagggccctcatttaactaatagaatcctttttcggtcttcgggcctgacaaatggaatactagacgagaaggacatagtcgaagaatcaccaagacagacagaagacaaacaggatgctgacgatcaagaaaatggtagtggtgaaagaaatgcagagatctcagatgtggcccctgtagaagagaccagggagctgtcacctgagcaatcaacagcaggtaagccatctgacggntcntctnccttggatagagcgagtcaagaggatgaaacctatgacttcaccacagattatatataaccgccacacactaacgtgctctctgtacgctgtgtagtttag

SEQ ID NO: 219 - DNA, 577 bp, Artificial Sequence
  Probe  1454703_x_at
gaactataatttccccatgaagaaatgttcacacgtcctacttcctgtcctagctccagagcctgaaaaggtgaacccactggggctggctgggggaaaagaggaaactttgttccagaaggaactgtctgagggatcatttttgtgcgtagaaagggaagaattctcncttgattaaagaggaatcaaaaggtgatttacttgttttacaggtctgaggaaaagaatgagctgcgtgtgaaggataggtaagcccttcaaatcattaaaatgggcggcgcagggttgggggaaactggttanctannnnnnnnnnnnnnntntnttntnnnnnnnnnatgtaggatgattttgctttntactcctaattcaaatttttatcttncaggaacagaaatcatgaaagatctgtttntccagnccagtgtttcaccataagagatcactttaaaataaatgatgaaataattctttgcaattncaaatttaccttccttgcttaccacattgttaactgtaggagctgatacctggttcttccactgttaacttgtactctgtgtttgttctagcaata

SEQ ID NO: 220 - DNA, 262 bp, Artificial Sequence
  Probe  1433674_a_at
tgggtgtacgctctcttttcagaatgtggttcatctcaaagccctttacactttggagccaggcctgttcaattttacaaataacacgttttgcttgtagtcagggtgctgtgacaacatgaagagttcgagctacctcccaggatgagtgcaggttttcctggtgacaaatctcaggcattcaaaggttctgttagcagtgccatacttgtgggtttacagagatggtgtgtctggctgagaacaaaggagccgtaccagg

SEQ ID NO: 221 - DNA, 446 bp, Artificial Sequence
  Probe  1433880_at
tgctccaccaggttatggtgcctgacagtgaggccctgaggataccaaagcagtctcacagaatcgacacagacgggtaaaccgcttgggaacctcgaggnaanaagaggctacgaaaaccttttcctaagaggctacaaatttggaagcagggaaaacccagacatgagatgtttttagtttatttctccagaagggggcactgtatcaattatgtgaagggacatgcagacagcctagctccatggtgctgtggggtaggactgaggagccctctggccagaccccagcacggccatgtctctcccaaggatcatgttcctggaggtcacgcccctggtccttntcataagtggctgtgcacagcagctctctggaggtatttggaacattctgctgtcacacatgggactgctncttcctgaagcccatgctgttcgtgggaa

SEQ ID NO: 222 - DNA, 324 bp, Artificial Sequence
  Probe  1455035_s_at
atggaatggaagacccacctgtctctctacctaaatccaagaaaaagaaagctcccaaggaggagttggccagtgaccttgaagagatggccactagcagtgctaagagaaagaantcctcacctaaggaggaagtggccagtgaaccagaagaggcagcaagccccaccacccctaagaagaaaaggaaattttctgaggagcctgaagttgctgctaacttcacaaagagcagcacaaagaaaaagaaaaantcccagaaagcacaggaggattagaacggacctgcttggtgggaggggcataccttgtggcagctcctgc

SEQ ID NO: 223 - DNA, 316 bp, Artificial Sequence
  Probe  1433996_at
gacttacttctagcttgatcttattttgaagcaatagataccattacaaaggttgtatatctgaacctgaaattcttaaaagattttttttagttgcacagaagaaaagcccctaatcaccttctctagaagtctatagtggctggccattgtgcttctttgaacacacagaacagtgactaggagggcccattggagtgccaggcagagtgagccactcagcgagtgggaggtccncnnctgagtatctcatctgagcaggantgagatccntgagggacacaanccccngatgacagtgtaattcaggtcactt

SEQ ID NO: 224 - DNA, 377 bp, Artificial Sequence
  Probe  1434033_at
tcccatactcagacagtgctgcagagctgtgcctcgctgtggaaagccccagccccacacagagagcaaacagtgagataacaggagctgtctgtcgcaaagcttgctttccactgacactcagttcaccctgccactctgcaaggtttgccttgggcagctgtggggtttttccgatcttgctagcccgcaggcatcttgacatcttagtaactgcctttttaatcccagtggcctgtaccatattatggagaactgttaactgcttaattgggtaattttcaaagaaagtgatgtttgttaaatgggncgaaataagaagttaaatttgaaagtgaatgtgttcaaatgaaaggcttgacaattgcatctgttac

SEQ ID NO: 225 - DNA, 535 bp, Artificial Sequence
  Probe  1434060_at
gctgagcgcttgagatatgccatcaacaactgccgttcaattgacatggacaactacatgctctcaagaaatgtggacaatgcagagggctctgacactgactactgactgatgagggtgctgtcacccacctttctcaataatgctcacttccaatttgatgttggtatacttttatggtaactacatagatgttttaggaacataagctgatacaaacagtggccacatttagttacttcaaatgaaacaaagaaattagatggttttatttttctgtgattgtacaaaacaaagagcagaaactgctcagtcaggttttcctctgtattttttggtcactgtggataagtttgcatggagccattttggtgtatttttagttgagaatgatacatttttgtaancccacccagtgaacatgaaattgtacattgtgtataattgttcattagaaaggacagttttacatgaatattcatatatttattttgttttagtttgattcncctgtgcagggttccttatgcagaga

SEQ ID NO: 226 - DNA, 553 bp, Artificial Sequence
  Probe  1434168_at
gaaggatgctagactcagctcgttttgggtttttgttttttgtttttccagatagggtttcttctctgtatagcnctggctgtcttggaactcactttgtagaccaggctggcctcgaactcagaaatccgcctgcctcgtcagcttgttttgattagtcagtctgaaaaggtgtctgaacagagtgcatgggagaactagacatgggcctgtgaacctctgcagaaagggatccagatggaaacacgccagaccctcaggtgggaatgagcaaggcagatgaagaggccaggcccctgaagctgccgtgaaacaggaggtttgctgaagctccagagccgaaggctgatgcggtaggcaccgagcctctctgctctgctttgtagagccaggcagaagaattgatctgaggtgctgatcctgtactcttagggcttcaaagatggaagctgcttcccactggaccatggaaagaagctgtaagcctgcagtgcctgccccgtgggtgccgatttcactgctgcctctgacagctggagctaggactcttttg

SEQ ID NO: 227 - DNA, 421 bp, Artificial Sequence
  Probe  1434193_at
tttgtcaggtccacagtcattcagtgtaaaagagctgtcttgtctaggcctcagtctcccctaataactatgttgctctgttttgagggcagcattaatacttttcatgtgacatgaaaacaggaagtaagtgggaaagaagctaaaaccactccaaattctaatgccccatgagtaatcttctcaatgttttggtgtgactgtgcctcgccatctttttctatgattatataaagatgcatatttttttaaatgaaatgagacagtatagcctattgtccaccgttaagatnccagagttcttnaacatgactcatgtttttatttcatcaatgttcatggcttttactctgttcatcatgctgaggatgaagaagtggctgaagcgctaggagcacttgctgcttgtgcaaagggcctg

SEQ ID NO: 228 - DNA, 518 bp, Artificial Sequence
  Probe  1434316_at
gagtgcgagctaaacagacaaacctctaatggaagtgtgagggaagctggggctctgctctctgtcctgtgctgtggatggagatggctgcttcattttttaattactgggttttctatcttgttatatgaaatacctttaatttatttaatatacccattgttttagatctctgccattttgagtacttgtcagttactagtatttatgtgcacggtgagtgtggttagaatattttatttgcagtaaactgnatctccaaagatttccttttgaaaacactttttcctcatccttaatttttacattcccntcttactaaagattaagtgttctttgataattttggtgctcatgtgttttggggacaaaggtgaaatgagtctgttgtcacaccagggtgtttgttttcaactcacagatccaatgtnccttaagaattggttttcatttagagttcagacagttgatagagttnccatttttaatatacatcgttggagatctgcttatttgta

SEQ ID NO: 229 - DNA, 557 bp, Artificial Sequence
  Probe  1434398_at
gaagccagttgtagagcatttaacaataaattgtgctgtacgtgtaaatgtctttatcaactaaatgatgtaaaattttctttaaagtaattttagtgtgtgtttgtttataacttctaccctgtgatttccaggatattggaagtgatttactgtatcttgtgacatatgacttttaaacaaattctagtcttcatgctgagagagcactactgagagagatgtcgaaaagtttccaaaaagccttgattcaatcagaagaaagaaagcttgaactgtttgttcttggtnccttgcagagagacntcagagcnactntccattgtagctttcacatggtttgtgcaccacacccaaggcaaccatagctgtggtagagcntggtaaaagactgaagatacactggtgctttgatgaaaaaggtcagttggctggttcccctctcaaaaagcttattactcccaaanccaactttgtaacatacttaaaactgctattttcgcttatttntggaatgtaaaaatgtataaaaataagagttagtgtatgcttcctga

SEQ ID NO: 230 - DNA, 428 bp, Artificial Sequence
  Probe  1434403_at
tgagaatgcgtgtccggttgcctctgtccctgtttgtgcgtccttcctcccagccggttcccaccctcctaagggttatgccagtggatctcacgcctctgtgcatcacctatctttggtttgtgaaggcagcagttaagggcacaaagacagccatggggacatttatgtaaatatgtttaattgccacactgcagctgaacggtgtgtagtattttcccagtcagctttgccatactgacgtcaatcatttgagagaaattattcagattttatttttatatccatggtaacagaacctaaccaaaaggtttcccgttcagaagtttccttagctctcntcccnagctattcgctcacattaacaagtcaaagtgcctgaagcataactcatttacctgtatactaaaacccctgttgtactgatt

SEQ ID NO: 231 - DNA, 403 bp, Artificial Sequence
  Probe  1455602_x_at
aaggacaatttgtatcaaccactttttgttatgcaaagaaaggttactcaatttgtagttcccatactactgaaaatcctggccactgagcttgnactctatgggattagatatataaggttttgttgaggattagaaattaaactgatccctgcagaaggggggtggggaaccatttaaattcaatacttacaaacttttgcttatagttttactgtacatttatttttaaagaaaaagataacaaaagccagaaaaatacaagttgtcccaacaaataatttcaaatctttgtcatgactgatattataaccttctacatagtttttttttaattaaagttatttctatatgccttgtattgtgtgatttgattaaatgcacaaacctttcgtaagctaaa

SEQ ID NO: 232 - DNA, 456 bp, Artificial Sequence
  Probe  1434574_at
ttttcctctccactctgacaacaggcaanagtgagggnccaacaatacagaggaagtaacgtgtcgnngctactcttccatgcccagtgccaggagcgacggtacactccagagtgctgtgccctgatgggcctttgagtggccagtgcagtggatcatcagccctgtctcccaagagctccagtcccttctccggagaggaagnnnntgggtccactggcagctcaggaggccatagcaggattgtcacgtgtgctcaacagtcctcctcggctttccagaagaggctggaacagggcccttgaagatgtttgtggacntcagagggccaaagacgacacaaagtgaaactggagacaccnccccccctnggcatcatgaaacnnccgatgtnactggctcatgcntgggagacaccagcatacaaantcttgacaccaacacctcagagggctg

SEQ ID NO: 233 - DNA, 553 bp, Artificial Sequence
  Probe  1434581_at
actgtgttcaagtcgtctggtattgggggaaagctgacagtgtgcgcttcataaacaacctttccctgggctttcctttttgtatctcaaggttctaccttaaccagaaccttttgaactggcagagaccaacgctctatacacatgccagctgtctaaaaggcactaaaataaatattcagtgctgggcaaatccgaacccnnctttcagaccagtgcttatttgtgtacaagtgagatttctccagagaagcaaatcagaaggaacaaaagcaagggactttgttgtttgtttctgttttttatttttatttttnagacagtgagtttttcctgtgtgatgtgtgtgctttctttgagtctgtatttcctttcagagagctctcttgtaaatgacaaactgtgaaatgggttgccnaaaaactgtntcctttgttagatgcttcagagggaactgtgtaaattctctctgcaccnttntcccatctgcttcctcctccctcccatgagagcaaggaccacatccaaggatgtaattaccatacgcttgcta

SEQ ID NO: 234 - DNA, 111 bp, Artificial Sequence
  Probe  1455643_s_at
gcagtcatctgcattcgagggctcagcagcgtataccttcacactaggtttcagggcctggggaaggaaggcacatgttacaatacagcctttgtctacgtaaaggaggca

SEQ ID NO: 235 - DNA, 494 bp, Artificial Sequence
  Probe  1434660_at
gtgtgtgtttctcataccatattggtatcatgatccacacagacataagcatccaagagaaggaggaagtgagagagggagagagagagtgtgtgtgtgtgtgtgtgtgtgtgtgtgtgtgtgtgtgtgtgtgtgtnnnnnnatacatacacatctgtgctgagtcagcacctcagtttgttaggatatggtgtttttcccagcactatgtgacttatgatctagccaccagaattcctacctcctcctagtcgtcaaattaattcttcggctcttcactcttctctgctctcacagaggaatcttaagcagatgaagacaatggaattaaagtgtccctaggagccacagttgtcacttggctggtagagcacatgcttggcatgcacgganccctgggttcagtatccagcacctcgtaagccaggtgtagctcgtgcactccaagggtagaagatcagacgggcaaggtgatcttgcatctatagactctt

SEQ ID NO: 236 - DNA, 529 bp, Artificial Sequence
  Probe  1434670_at
gcttagaactcactcatttgccagaggggtccagtctgctccccccccccacacacacacacactctctaaaaccacaactcaaaagaaaggtcaggaggtgcagacaaatcgcacacctcagacacagtgggcaaggaaggaaaagggtgttccctgtggctcatgctggcaggatgaggatgcttgggattctgagggagtttgcatgatcttgtcaccaaacgatccttttcaaaatacaagtgaggagatgtggaggcgtgcagcagagcagatgaggaggggggcggcagtggccagccttccctcctcggcaagatgtaagggacacggccatttgctatgctaaagaaacagcccttcaccggaaggttctcaccacatcctcccccttaccttcactacatccctttcagagagccaggccaccaccagggcccctggcccgcccacttacatntgccaaagtgaaatgttgcatgccggcgtggaagacaaaaccaaaccgcacaaatcccagtgtgtat

SEQ ID NO: 237 - DNA, 514 bp, Artificial Sequence
  Probe  1434745_at
gtctaagtaagccagctgacctctaagcccaaaggactcccccttgcttaccttagacagtccaaccttgggaatagctttaaaaaggggaattaatactgagacagtgaagtccccttaagccgacaagccctccataggtgcacaccgcacacataggcttctctcagtgcagactccaggtggtggtcaacagctgcggcatgcttatctatttgtacgaaatagtgtagggaagccccaggaggtgttatagcttccntataccatgtggcttttgatgnttctggattntcaacagcacaaaagagtcctcgagccggaacatgcgctactccgnttttaaacgtggctgggctgtgagacccgttcttggctctggttttacgaagccatcaatacatacacacccttgatggagctgcagatccgttccttttcctgcagcccacttgtgggggaaaagtgccttactgacttacagtagccactcagtttcaacaggtgcctatct

SEQ ID NO: 238 - DNA, 213 bp, Artificial Sequence
  Probe  1434773_a_at
aacaccatgagaaggacctcggaggctgagaacttaactgctgaagacacggacactcctnccctgctgtgtatagatggaagatatttatatattttttggttntcaatattaaatacagacactaagttatagtatatnctggacaaacccacttgtaaatacaccaacaaactcctgtaactttacctaagcagatataaatggctggtt

SEQ ID NO: 239 - DNA, 572 bp, Artificial Sequence
  Probe  1455832_a_at
tcacgtcgtgccaggctcaggagttgtgaaaggnttacaggaagtgggtctgcctttacatcgagcctgcntgntcattgcagaaatgagctctgctggctccttggccactggaaactacaccaaggcagcagtcgggatggctgaggagcactgtgaatttgtcattggcttcatttctggctcccgagtaagcatgaaaccagagtttcttcacttgaccccaggagttcagttagaaacaggaggggatcaccttggccagcagtacaatagtccccaagaagtgatcgggaaacgcggttctgannnnnntcattntnngnccggggaatacttncagcnnctnancnncctagaagcanctgagatntacaggaagncntgcctgggaggcatatntgagtaggcttnntgttnncagtgagacagaagacactgaaaatgggtggtntcccaaaanccnctggcttcagagaccagtgntcaggnccctgcacaggntctgtgaagagagantntgcagcattgtatcatggtctaacttgtaatcactgcattggtactgtaat

SEQ ID NO: 240 - DNA, 154 bp, Artificial Sequence
  Probe  1455841_s_at
tctgtgtcactccagtggaatttaattntntntnntnccttctgnccnagttagatttggttgagatccaagtaggganccntntggggtnccctcctgtgctgaccactactgtactggaggaggntctggggcttctgctgtaataaaggac

SEQ ID NO: 241 - DNA, 241 bp, Artificial Sequence
  Probe  1455898_x_at
ttcaccatcctatccctaaatcaccnnncccctnntccacnngtnactggatcccactntcaccctcgntncttacacatttcagacccttnagncnaacccttgcatagagtggtaganccatatctcagtgctcggtttcagtattcaactctccactccctgtgnccagttgggatttaaaaactcagattgctatggaaaggggctttgctgggaaaggcgactcctctaacttcac

SEQ ID NO: 242 - DNA, 417 bp, Artificial Sequence
  Probe  1455904_at
aagtctgtcacttgtttttgtacagattgtaatatagcttgtaaccagtgatgtgatgattctgccaaatgatacaaagtgatatcacctttaaaccgttccattttatttctgaggttactatactaacatgtaaaatttaactactctaagccagagaagtaagtcagtctttgttttcttagggctcctgtgacaagtggacatgcagtgactgcacctttgtttntgaggtgagaactgcaaatgcttaaccgggaacctactccagaatacatgatgatctcacacaacttgaactctctcactgattacttgatgatagtaaaagatctgatgttctgtgttttaacagttaccatttaagttaaaattgtagaaaagtgtttaacagctaccttctgttggttgttgcag

SEQ ID NO: 243 - DNA, 204 bp, Artificial Sequence
  Probe  1434859_at
atttatgtcctactgaaggtggcagttttatgagttcctcttgtaagttcctctttaaactcctgagttcctctttggcggtccatcagtgtgccaagattgcactatggacactcctgtgagggcattttcttgtttaataatcctgtgaggtggaagagcccagtccacaatgggcagtgacacccctattcacatagtctt

SEQ ID NO: 244 - DNA, 91 bp, Artificial Sequence
  Probe  1455956_x_at
tggaagatccggaccaagccaccacccctacagacgtgcgggatgttgacctgtgaggaagccattcgggcggcaagagagaggcgtgttc

SEQ ID NO: 245 - DNA, 270 bp, Artificial Sequence
  Probe  1456066_a_at
ggaggaagtgaaccattacagcaatgagattaacaaggcatgtatgcctcttggcttgcacagacagttccccgaaaacaacctgcagatgatggtgcagtcaggagccaaagggtcaaccgtgaacacgatgcaaatctcttgcctgctgggccaaattgaattggaaggtcggagacccccactgatggcgtctggnaagtcactgccctgctttgagccttacgagttcacccccagggctggtggctttgtcactggaaggtttct

SEQ ID NO: 246 - DNA, 99 bp, Artificial Sequence
  Probe  1456071_a_at
taatgagtaattccactgccttatttattacaaaacaaatgtntcatggcttttaatgtacaccataatttaattcacacaccaaattcagatcatgaa

SEQ ID NO: 247 - DNA, 557 bp, Artificial Sequence
  Probe  1456117_at
tcaaagcacatctcctaccaaaattgctcccaagatggagggtgagacctcagtgctaggttgacaggtgggcgtcgccatgcctggaccaattcctgcacagcacaggaaggctccactccatgaaggagtgcatttagaatggcatcctggtcctggatgcagattctcaagtaaaattgctgttaaatttcttcaagtccagagaagcaaatcttagtgcgggcagaccgagttagtgtttgatgcgactatctccatccttagtatggatgtggtcagttctgcacaatttggtttcaaactaacttttttttctttcctttttnaaaantaataataatgtctcagtatgtatcctatcccacactggccccacaccggtggtctccctgcctcagccaggctactgctgggacacgcacaggaaggagcatgagggggggttgggaagcagtcncctgcctgggaagtgggtctgtgttttctctaactgccacagtgctatttgtttaccatcaaattgtattggtgtttccagggcttgttggtacaaa

SEQ ID NO: 248 - DNA, 473 bp, Artificial Sequence
  Probe  1435339_at
ttatctcaggaatgggcccttctaccaagggacccatctgtcagcatcactctctgggtccccagctcagggaaccaccctccaccctagcgccagtttccccattcatatgcacaccaagtgttaatgaaatgtcctaaactagcctgtctgtaagacccagacccaagcttacctagtgctcttaaccccagatttccaacaaggactggtcctgagaggtcctcacagatctgctcttctcccagacgacagggccaaaagatggataccctggccttgtcacatctcccttagctcctcccaagtggaggaaccgcaaagaactgacagatggggtgccctggccggcacccccttgtgtataacgcctgtagacttgtatatgactcctttaatattgtaaagatgctgatgtacaattgttctgtgtttgtgtgaacacaccgcgttcccggttcatgccataaaga

SEQ ID NO: 249 - DNA, 576 bp, Artificial Sequence
  Probe  1435345_at
aaatctgactgttattttgggctcagctagttttctgtcctctggactcattccaagggaggagaagcnctcagagacagagtccactcctgaaggtctcagagtgaagacacnaagacccccaagacttttttttttttttttttttttgtgacagggtttttctgtgtagccctggctgtcctggaactcactttgtacaccaggctggacttgaactcagaaatccgcctgcctctgcctcccaagtgctgggattaaaggcgtgcaccaccnactgcctggctaccccaagactcttgactctgcttgcaaatttagagccagtgggctcagggacagggaggcctcagcctttggggtttcaagctggctggagcccagantcctgccctntgaggacccaactactttgcctccatctacctccnccttggtgattccctccctgcctcacatctggggcccattagcaccctctttcctggttggtggaaagtgcaagaagatgggaggcatacctggtgctcaacacncagtagnccttcaataaaagccacttgcattagcatttta

SEQ ID NO: 250 - DNA, 502 bp, Artificial Sequence
  Probe  1435544_at
agagttggaccgatgctgtgcgcctgggcctggagggctgccagcgcctctacccagtgctgcagcagtgtttggtgcgggctgcccgnncggagggntgccgccgccccaccctgattggaaaaaaccggagcgacgaggattcgaggactgctatcgcccttctctccctaaaggaccaagcgctgatggtcgcctgactccgcagagctgagaaacgaagagtggggcagttgggcgcacagaaccaaagcactggacagctctgttggaaaggtttaaaagnccgttggaaacacgatccagttggagagatttgaaancagccatactcccaanttggaaaggacttgggctggatttacctagtaaatgagataaggggctatgntnttgtaggagatggacccagtgcaggccagccattggctccaatacatnataaactgattcttttgagaggatggaccaggggactttgccttctcactactcagagtaa

SEQ ID NO: 251 - DNA, 351 bp, Artificial Sequence
  Probe  1456672_at
tgagaaaatgccctcccgagtgcaggccgtcctcctgatgactcaagctatgtcaagtagacacaaaactggccggcacacccatcttccctcactcttttttgtagctctacctgtgcactntaattcccactcgccctttgcagtgttccaagatcactggacctgtcagcccagaagcagaggaccctagtactgtctgcatcctctagtacatggagtatgctttgtcatgattactgagttctggctcctatcctttcctgtgagatgaaaccaaggtacacacttctttccctcaaggcagggcttttctgtacagccatagagggcctgcctgggactcatgct

SEQ ID NO: 252 - DNA, 467 bp, Artificial Sequence
  Probe  1435655_at
gtacttgctttctgcagttaaacacagtgggaacattacttttgatgagattgtcaacattgcccggcagatgagacaccggtctttggctagagaactttctggtaangagcacgctattttccctgagtggtggctgccactttgctgttgaaggttcaagatttggattttcccatggatccatattttttnccctngtgttcctgcaatggaacccntggnaattctttcccctgtgactgaggccaccattcttactgtaggaactatcaaggagatcctgggtactgcacagtctgtgggctgcaatgtggatgnccgccacccntcatgacatcatagatgacatcaacagtggtgcagtggagtgcccnnnnngtnannnannnnnannnaannnnnnnannannnnnnnntnggggnaaagctggacttttgttgctttacttttggtgttttactgg

SEQ ID NO: 253 - DNA, 75 bp, Artificial Sequence
  Probe  1456738_s_at
cctccttattcccaggttgaattgggacttacccattcagcnagatagaacctgctccttggttagctttttcaa

SEQ ID NO: 254 - DNA, 187 bp, Artificial Sequence
  Probe  1456865_x_at
tcattttgtttgtaaccggtggcttttgtttaattggcttgggttttagatattntcnaaanatnnntggcattcantatggaaccaaggctncccntggaantcagggccaagtgctgagattataatcgagcagcagatttcatgtttatttctntcctagatgtttttccctgtttcattgtct

SEQ ID NO: 255 - DNA, 342 bp, Artificial Sequence
  Probe  1435998_at
ttagcttagggcatgggcatttcatgaatctcagtctctacgtttgtcattgctcacacaatcacattgttgcacagatcctgattcagaccaagctgtgtttatgctcatagaagcctcagtgttatgtttccctggagctttggattgattctaccgtgtttatgttaaatgtgtgcattgatttttctctgctagtatatttctgtaaaaataatggtcttggaaaatgatggccaaaaatgatagtcttggaaaatggtggctttccattaacatgtattttaacagtcatgtattttcatttgtgttactttgttttggaggtgcacttatgccagt

SEQ ID NO: 256 - DNA, 479 bp, Artificial Sequence
  Probe  1436007_a_at
ctgtaaacccagctgtccaggtggggactgtaacataggttgggaactgggtttgtgcttaaatcttaatncacagagctagtgacntgagaatatatacttcgcatcacacagctagaggcttgcttggtaagccagaccaagaagatatgggagaancagagaataggcagtaccttgctaatgcagacggggctagaccatcctcaggtgtttgacatgctacctcgggtctgagattgaaaccatcttgaaaggataggccttagggtcacttaggaacttagcagttagttttaaactatagcagttaggacagaaaataaagaagcagaaaccaaatccccntgctgacgatcagcatgcctgtcctgctctgcagagagaggagttaagcctgggcctggaggacacgtttctccaaagctgaatacagttctcagatatttttactgcattttgttcttggtacagctgaa

SEQ ID NO: 257 - DNA, 413 bp, Artificial Sequence
  Probe  1457083_at
agacgggagatctttgtagcctggctgccctgggacaccctctataaaactggctggcctcaaacttatagagatccacctacccctgtctcaaatgctgtgattgaantgtgtacaataccactggccttaaacttttctttttagatttatcttatggatatgaatattttgccagcatggatgcatgtgtatcatatgcccatggagaccagaatcagaagccctggaattgcaattacagtttgtgaactagctgccatatgggtactaagaattgaactctagtcctctgcaagnatagcagttaagtgctcttnactgctgagccatctctccatntccagantactttataaatntcacacaactcacaaccaccagtaactccagctccaggaggtctgatgacc

SEQ ID NO: 258 - DNA, 349 bp, Artificial Sequence
  Probe  1436222_at
tgcattgtcttacggatgtttagataggtgtgttttggacaacactctgggttcttgtaatgatgttgatcaaatgtctgagctgaaaataacttgtagacaatttttaacactgaagaaccttgtggcaggcaactaagaatagtcgaatgatgtttagcatatttgtaagaggtattttcttttaattctagttaactggttgcatgcttgttcatttggctggcttgcttgggtaagaagccattgggggccttgaattccctccagtcatttgcagtatttactgcataacatttagcttttgaacatgcatgtgctaccatcagtactaaatggtgccttaaat

SEQ ID NO: 259 - DNA, 234 bp, Artificial Sequence
  Probe  1436887_x_at
acgcacctcattatggcctttccatctacttcnggaacttnaagggttttgccatagctggnccaantctggntctgtgtcactccagtggaatttaattntntntnntnccttctgnccnagttagatttggttgagatccaagtaggganccntntggggtnccctcctgtgctgaccactactgtactggaggaggntctggggcttctgctgtaataaaggaccacaata

SEQ ID NO: 260 - DNA, 427 bp, Artificial Sequence
  Probe  1436892_at
aattcctcgagttgctttgcatcctnttggancagggcttgnaaggnnatttgggtttntgtgagcttgcccagttgacccgaaaaataggactttttagaattgcattgccacttctaaaatcctagcaatacttagnataacacatcagtgagataaatttntactcnattattttacnaaccnnattgagttattcanacacnanccagtattctgtgaacaagttccagatgaaaaacacagaagttgtgaatgaagtngtanccattccctnccctncactatatttttcagggtgcattttaaatactattgattcaaacagtggtggcttgcgttttttnnaggtatatatttttggttagttttttggggggggttgttttgtttttttaatttgaggctcaattcttcatttatactt

SEQ ID NO: 261 - DNA, 154 bp, Artificial Sequence
  Probe  1437007_x_at
tcaaagaacagacagttcggtagaaacntcagntnnctggagaccctaggagtaaacgtgaantcagcagcanccnttctntcataggtagtggctttcctgtagctcccttcatntcctgtgggacgtgtgcgatggattaaatatcctcctg

SEQ ID NO: 262 - DNA, 41 bp, Artificial Sequence
  Probe  1437052_s_at
catatctcagtgctcggtttcagtattcaactctccactcc

SEQ ID NO: 263 - DNA, 121 bp, Artificial Sequence
  Probe  1437238_x_at
tctggctgagatgcttgaagaccttcatatttctcaagatnccactggtgaagaaggtgcatcaatgatntcatgatgagtcatattatatgttgcttgcatatctgtgccctcaggaagt

SEQ ID NO: 264 - DNA, 342 bp, Artificial Sequence
  Probe  1437592_x_at
acataggcttccctggtagtaccagaccctttgactccagagtagagaggaaagctggagaggtggggagacacggctagggctcagggcaangagaggctnccctggtctagaagccccttgncccttccngtgtgtattctcancctcaggatgantactcantcctgtttgaagacacntntnatgcagatggctanntcccctcccntcaatgtggcccagaggtacgtggtggcttgtaaggagcccaagaaaaagtgaatcgggctggtaggcttgaatctgccactgccaacctcatgggagaaggctacaaaggagttcctatgatcaaataaa

SEQ ID NO: 265 - DNA, 569 bp, Artificial Sequence
  Probe  1437630_at
tttcgtgtccaccaaagcagagatgatttgcaatgggatcctccctatcgaccagatgagagaccacgttccacccgtatcactagtttgccagaatatcccacggcgagttttggaagtgacatatggtattaacattataaagcctagagaagatgaagacccctaccgacctccaacctcagaagaactgttgacggcttatggatgcatgagaggattcatgacagcacatgggcagccagaccagcctcgatctgcacggtacatcctaaaggactatgttggaggtaaactactgtattgccaccctcctcctggaanagaccctntgncngttccagcaccaacaccagcagntcctagagagcaaagtaaaaggtggangaactgagacttcagccgggcaaagnccgaaaanccaaacagatcgaaaatgtantcgacaaaacttttttccatcaggagaacntcagagcactgaccaaaggtgtnccaggctgtcatgggttacaagcccgggcatggcctggtgactgcnggctgctgcgagcgctgagaatgtacct

SEQ ID NO: 266 - DNA, 170 bp, Artificial Sequence
  Probe  1437658_a_at
agtatgctgaactataatttccccatgaagaaatgttcacacgtcctacttcctgtcctagctccagagcctgaaaaggtgaacccactggggctggctgggggaaaagaggaaactttgttccagaaggaactgtctgagggatcatttttgtgcgtagaaagggaaga

SEQ ID NO: 267 - DNA, 588 bp, Artificial Sequence
  Probe  1438015_at
gatgctcagtggctgtatgataaccataaggatgagagttacttgcggcgtgttngtttaccctttggaaaagctgttgacatctcataaacgtctggttatgaaagatagtgcagtnnantgcaatctgntatggggccaagatcatgcttcctggtcttcttcgatatgaagatggcnattgaggtcaaccaggaganttgtggtcattactactaagggggaagctatctgcatggcaattgcattgatgactacagcagtgatttctacctgtgaccatggtatagtagccaaaataaagagggtgatcatggagagagacacttatcctcgcaagtggggtttaggtccaaaggcaagtcagaagaagatgatgatcaagcaggnccttctggacaagcatgncaaacccacagacaatacccctgccacatggaaacaggattacattgactatagtgattctggcaagaacacactggttaccgaagcagtacaancccctcagttagctgctgaagcagtaaatgtcataaaaaggaagcgggatagtgagagtgaaagtgatgagaccccaacagttcc

SEQ ID NO: 268 - DNA, 323 bp, Artificial Sequence
  Probe  1438016_at
catggcatggcctagtaactatctgcagctatttactaatgagcattttcaacaatgccatagatagtcctctcctactttgctttttaaaaagttggtaaaaattaaatctgctacataaaatggtatagaaccttctgtgagagaaaaaatgtaattgaactatgttcactgggggactcttatctccagtgttctatgaggactgctctattcaggtattaaccttgcctggtggtatctatgatatttaggctagtactctgtatttttgtgtacaacaggatttctctatatggctacttgtgtttcttatggaagcc

SEQ ID NO: 269 - DNA, 418 bp, Artificial Sequence
  Probe  1438038_at
atttaggttggaccgaacagcaaaggtaaactaacttgcattcccaagactgaccttgctaattagtgtgaggaataaggtcattaaaggtgacgtctgctaggatttgagtgcctctgctgctggactgtaaatgttttctgagtttncatgtaccttttgcatgccttagaagaagcnacatagtaaaaccagcatagtgttctgtaatgtagaagggtttttgagaaagggccctggcacagaggagaggtaagatggagcatatgggtctgtgtgcctggtaacatggcactctttggacaaatagcaaagtagtaatcaaggacttgtcaataaaatatacttgtgttagggggaaaaaaatggaagctgaatgaactaaattacaagtcccattgggtcagaaatcttgcat

SEQ ID NO: 270 - DNA, 564 bp, Artificial Sequence
  Probe  145910l_at
gagcccaaggataccgatcagaagcctgacacaccaaggccattgaggtaaactgctcccaatacccagtacaatgggaacatccaggtaccaaagccatccagatggctaaaggccctcagaaagaacatgntncaacaaacanttagggcnatatgacnaattcngagcacngctgccctaatacagcaaaccctgnatactctaacacaactgnagcacannaaaatgnccttaaatctaatcttntgangatgataaaggcctttgtatgggaanggaataaatncctgtaaagaantacagattacaatcagagaggtagaggtctttaaagacggaaaaaaaaaaaaaacgctgaaagatatacaattaactaggtggtggaaataaataaaactgttcaggacctgcaagcagaaatagaaacaatgaagaaacacaaagtgatgaattcctggagatggaaagcttatggcagagaatagtaacactagagtgatacaagtgccaccaacagaagacaggtgtggaaaagaaaaccacaagcattgatacatcagt

SEQ ID NO: 271 - DNA, 194 bp, Artificial Sequence
  Probe  1438155_x_at
acctgtactggtgctggattctgcaagagantccgacacacctcttganccnaccctgtaaccagagactgctcaactctgggactttattattttataactgagantcatctggancccgaccccgaccccgacccttccctcctgggttggatacatctgacggacaacaggggcggctaccaaagtggaat

SEQ ID NO: 272 - DNA, 472 bp, Artificial Sequence
  Probe  1438198_at
cagcattggtgagcctggtgcgcacctggagctacaaggaatttccaaggcgaccctctcagcctctttaccttcaagtcctgggctcctgagctctgcccccagcatgaaagctgctttcaaagcaacctgtgataattctgtaatgtttttgagtttgaccatttatgtacttgggctttgggctcctcacctgtgaattgtagatttctattttgcgggttttaacccaatacccttgtcactggctggcatcagcaagcttcggcctttttgcttccttctgtgttttgctctttggtggtccttccatagctaggacagacacggagaacctgtctttcagtcctgatggcacaaacagcacattgttgctgccaccgggacagctacggcagccagtggcatgcccgactctccgcccgatgtctctgcggatttcaagcttatgctgttgtgtcagtttcctgtc

SEQ ID NO: 273 - DNA, 181 bp, Artificial Sequence
  Probe  1438415_s_at
agctgggtggtttgtacattcaagtttgaggtgaaccctttggtgggagggggctgtncccantntgaaggcttgacaggnccacccctcacctagaacctntgggcctaggaacaggggctgctggcttggcccgnccanccgggtttntcagagggtctgtgggttgacactggttctt

SEQ ID NO: 274 - DNA, 425 bp, Artificial Sequence
  Probe  1459557_at
ttatctcaaggccactcaagttgaccattgagattaaccatcccagaagaacacacataaccagcagctgattggctcctgggataaggcaaactgtgtttccagctctgatgttggcatcctgcctttcctggcccgtgtctcacctttgatatgtcagtgacacttaaaaaaaaaaaaagattgaggcgtgcccttgactctgatcttcaggtggcaaagagagcccttgtttgaagggctcctcatggaggctggctgtgggaagaatgcaggaaggtagcaggtaaagccggaggtttaaaggggaaacactaccatgcctcctcgggatccctgccagccagccttgccctagacctagctttagaagggcatgctggacacctagaagccatgtcaggcggatgggatccaggcctcaa

SEQ ID NO: 275 - DNA, 457 bp, Artificial Sequence
  Probe  1438527_at
gggttcatagaacagtcagtcctaggaatccatgacactgcctaacccaggtttaattaatgccctaaccctttcccaggaaatttttgttgttattgttggaaatttttaaaggttcaattataagtagtttaaaaaaatttgagtatactgtagctatttcctgacaccactgagggcatcagatccctgtacagatggttgagagctctaccatgtggttgctgggaattgaactcaggacctctggaagagcagttagtgctcttaatcactgagccatctatctctccatctctataagcagtttttatgcattttgagataacagcagcatagcatgcgctgccttggagttgtagcagtcctgtgtctgcctcaggattattaggattacaggcctgagttgctaccatacctagatttagtagtttaagatgcttggtaatggccgggc

SEQ ID NO: 276 - DNA, 189 bp, Artificial Sequence
  Probe  1438630_x_at
ggccatggagaaagctgacttggctggtntggtacagagaagccagcttgtttacatgcttattccatgactgcttgccctaagcaagaaagtgcctttcaggatntattttttggaggtttattacgtatntctggttntcaattccaacagtttaatgaagatctaaataaaatgctaggttctacc

SEQ ID NO: 277 - DNA, 398 bp, Artificial Sequence
  Probe  1439027_at
gaagctgtcatgcactatacattgtgtacagttaaaagtatatatatatatatattcttactgagtgaacgcctcctctccccacgcctgtatgtcactagcatctaaggagaatgctcaaggcccagtgctgctgctgctgtggtttatatgggttttgttctgttttgtttttgtgtggtaaattgatatttaaaaacaacaaaaaccacgactactgtttacagactgaaaaaaaacaatcactgctttttatactactgagatcctaagtcaagactttgcaaagcaggaatcgggttcaagttacttctttgctgtggacggatantcctctgtagtatctccacatgatggagagtgcacaaacctaggtgtgctgccatcaattttgtata

SEQ ID NO: 278 - DNA, 486 bp, Artificial Sequence
  Probe  1439071_at
tgttaacgctctggcatcatgtctacgccagctcccgcgcaatccggcgccatcgcnntttgggcgagcatggccatggggagtagagaggtggaaaccgagctgacgcctgcctgaccctggcgatggtggcgcgcgcngggaaggactgattggagaatggcgccaaatgggtcacggatggcgagggggcacaggagctctcgatgtgacctggcgctgtggatgtgatgcacagcagcttgagtgggactgctccgctgtcctcatgagagaacgctttttctgaagatgggaggtggctaatgaaagaaggaattgtgttctcggtgatgcctggaagagatgcgtgacgttggaagatgattacgaaatacaggaaaggaatgaaatcnaaggatnttttttgtaagagtaacacttacagaaatattcattaatttgaagatttatataaggcctggtgacctccgttaattcctggga

SEQ ID NO: 279 - DNA, 82 bp, Artificial Sequence
  Probe  1439079_a_at
acttacatacgccacggccatgcgtgcacctacacatattcaaatatacacacataaagaagtcttatttaccagcagcagc

SEQ ID NO: 280 - DNA, 172 bp, Artificial Sequence
  Probe  1460257_a_at
gagacggaagtaatcatcaaggacattttcaaacaaggcaaaatctgcttcatccctcngtaccaattccagagcaatcacatggacatggtgaggttaacgtcanctgaagaaattgctttacttcccaagacatcttggaatattcatcagcctggtgagggagatgttc

SEQ ID NO: 281 - DNA, 94 bp, Artificial Sequence
  Probe  1439399_a_at
ttccttgcttaccacattgttaactgtaggagctgatacctggttcttccactgttaacttgtactctgtgtttgttctagcaataaatgctct

SEQ ID NO: 282 - DNA, 384 bp, Artificial Sequence
  Probe  1439857_at
aatactcttccattgcatctacaggcagtggaaggtcagcaaagggcatcgaatgtcctagaactgcagttctggtagttgtggactgccttgtgagtgctgagggcccattctgggtcgtccataagaacaaccgtttctcttaactgatgagctagctagctctccagtttccattgactatgttttacttttcaaaatgctaataaattagaaaataagtaaattcaccttcctcctacctcaaccttccaagaagctatgattacagacacctgtcctctgaacagaaccttgccaggttgacattttttgttaatgttgggtttttttgagacagagtttcactctatgtagcctacctagcttgtgtctcactgtgta

SEQ ID NO: 283 - DNA, 519 bp, Artificial Sequence
  Probe  1415733_a_at
gcaatgttccggatcgagggtctcgcgcccaagttggacccggagnagatgaagcggaagatgcgtgaggatgtggtctcctccattcggaacttcctcatctacgtggccctgctgcgagtcactccatatatcttaaagaagctggatagcatctgaggacgggcagtcaggtggatgcatggcatgaagacctggtgacggcttctcctcctgtctgcagctgaattggcccagaaaggtgtaaggctgattaaacggacatataaactacttgttgagaacaagtaggactttgagaactgacctccatagctgaaatataaagccacttgggaagtgcaaaaagatgtcttgtgttcatggtgcccttaggcccatgaactccacccctgtgagtgttgatggagtggttaataatgtcaggctnnnnnnngngnnntnncnnttnngtttaccacttctgttcaaagtgttatttgtgaagtctgtttggaagtcagcatacacccacaatga

SEQ ID NO: 284 - DNA, 414 bp, Artificial Sequence
  Probe   1440120_at
agcactgtgtgacatctgcctaatgaaaagtaatgcctgtgtatatagcagtttatggagtgttctaattgctatgtggtccaaggtaattctcttgtgagagccagtgcaggtggcatctagccaccagatacctattaggagttaagtagctgccatgtatcatgaagaccacagtgtgtatagcattgtagtagaacactcacttagtataaaagagatccaaatttagtccctagcaacaaagagagtccttccctatgctgctagcatccacttggcttcaattctgtaatctttgttacaggatttctggacaactaaatgacaagtagactagactagcctcagagagcacagatagcctgcctctatatcctgagtgtgggattaagaggtgtgcactagccgggc

SEQ ID NO: 285 - DNA, 357 bp, Artificial Sequence
  Probe  1440205_at
caactggcctgcaagctaggctatatagccatattcctgcttcagttccagggctggatacccaggtgtcctatgaatttgcttcctggagctgctgtaacgaagtgccaaaaactggatggactaaaacgaaattgttgtctgttgggtctgtctcatggtttaatcaagacatttgntaccgcgnccactgcaagngctgggncaaaagggtgctgcaggtctgtcttgtagcattctggactcacttatgcattcagtgtcttagacttcaaaggtacacattaagtcctctgcatgtttccaaaatttcctttacatgaaggctatattggatcagagcttacctggagcagt

SEQ ID NO: 286 - DNA, 351 bp, Artificial Sequence
  Probe  1440533_at
aacatggaacccttgtgtgcagctctttccctcagtctgatttcaaggctcacacatgttgtagcatcatctatgctttattttatctctgaaaaactccttgtataggtagatcagattgtcagttcctcacttggcaggtatctgagttgatttcatttgtagttattttgaatagtgctgctaaaatcattcttgcacagggttttgtgtaaatatgtattctgagaactctgaggaatgnccacntcccaggatcngtctgngtttacttttcaaagatgttgttgagtaatagtttaatttctatcttaacttctcattatgaaagtaacatttggccaggcatgg

SEQ ID NO: 287 - DNA, 454 bp, Artificial Sequence
  Probe  1440897_at
taaagacggcactccagcttacaggatctataagccgtgtttcctgaaaagataaatgatttcctaatgaatggatcctttaggccataaaaaatccatttattttcttttggatttgaatagaggggatttgcattttttccagtgcagccccatttaaattaaaatgcagagcatgaggctagcctacaatgcaactaaataatagtgggaggggccgctgtattcttcctaggagctcagagtttgctagctttgccataggaaaaaagcagacactggtaccaacacctactgttgcagaagagaataccacaaggccgttcgggacaccttcgaggcgtgcgagaagctgggcggttggttgtgatcatgtttcgtttattcaggaaactaggcaatgtgtttatctaagagggaatctgccatttcgaaaacggctgcagttctgaca

SEQ ID NO: 288 - DNA, 446 bp, Artificial Sequence
  Probe  1441319_at
acaggagtggtgatacaaacctctaataaccccagnacttggtgganagagaaaggctgtgagtttgaccccaatctacatggcaagttctaggcagccagtgagctacaaagtcaggccctgtcncaaaaacaagaaaaaaaggaagatgtttgtgccaaggatggtggtacacacttaatccnccacttaggangcagnnacaggnatatcagaagctggagggntacatggntntcnaaacannnnncgcaaaacctaaaattgtagggggntggcgctcagcacttgatgttcttccagaggaccttgattttggtctttntccccgccactacccccaccccacacccacacccacacccatggctgctcatcaccatntacagcctcccatccaggggacccagagcctttatcaaactcatacacccagatgcagacac

SEQ ID NO: 289 - DNA, 557 bp, Artificial Sequence
  Probe  1441415_at
taattgggaggctgtgcttgcctgtntaaccttctctcttttcttcactcactaaatttaaacaatgcctggctagtggaggcacatgccttgtttatttgagtatattaaacattcctatgtgtgttgacagactgttcagcgtctcctcatatttttaaagnacactttttaaatccttcttntccggnacagatgctctanttaagtttatatctgtgctatgtctnccttgctggacgggctctccgcctggggtcagnacctntactaccgctggctgcccatacatttgcctgagatnaaacactgactacacagtcccctctntgntgtaaatcctncggtcccttagagagaacatctacagatctggaattaccaagctagcgagcaaggcgggcttcgttcctgtttatttatcatagancctatagtaaagccttggttttcttcaggtgtatgaattttaaactcctggtaaacagatttatttcatttattggtttcagcacatttagaaaacttggaaggagtttgctcagtatgttctgttt

SEQ ID NO: 290 - DNA, 311 bp, Artificial Sequence
  Probe  1441802_at
atcaatgcccgtgtgggttacatggcccctgggctaatttgaaaggaaaagaaacaancaccagncanggagattntgttttgttttttttnntnnctnntttaagatngttttacnatgtgcttctagtatntngacatnaaaatntnttntatnanctnnncatgntngcatacgcctgtaatcctgatttggggatgctgaggtaggccagcctgggctacatgataagctccaagccaggttgagctagaagtaggagaccctntcttaacaaacaaaatattcattttaaaactaggtgtggtggc

SEQ ID NO: 291 - DNA, 293 bp, Artificial Sequence
  Probe  1441811_x_at
tgctcttgggtaatctgggtgctgctgctcctggcttntctcacccctgtatgtgtctacatctggaaaagatttttcacaaaggcggaaacagaggagaagaaactgcnggntncagctntnatctancctttcntcttnctccgggcntccctcctactgaancctgnaagaagaatcaggcaggactaagaagaccctcccccactagcagggccatggccactgcctggttctgcccagcaccacagcagctctcagcagcacttgcttgtctttccatccttcaccgt

SEQ ID NO: 292 - DNA, 119 bp, Artificial Sequence
  Probe  1441885_s_at
atatattcagtggtctaagtatagaacccaatatgaagcccacaccctccaaatncctattgtaacttcccagaagttcactgaggtatacaaaccttaggtctaataaattcactgtg

SEQ ID NO: 293 - DNA, 434 bp, Artificial Sequence
  Probe  1442315_at
gagtctccacaatccttgaagaaaccgcagagcccatttccccgaggaaaatgttttctctctggtgtttggctgttttttattttattttttattttaaaaaaagccctctttggcttttcccgtggtccataggcccagccatccagggctagcggtcctcttggccttctcctctgccccaggccctngtgatatttattgtagttaaaggcaatgagagtgggtgggagaagagggtctggagaggtgggaattgggtgggcaagggcacagagctgtggcngnggnggggnntcctatngttatatgtaaatatctgtacagcgggctgcccccctccccanngcattctctgtccactggtgtgcattgatttagacttctccacttagcggagactctgtgaaatcgtctgatggtctgtatggaag

SEQ ID NO: 294 - DNA, 484 bp, Artificial Sequence
  Probe  1442427_at
tctaaaactctcttatgctgcacgcctctctttgagttacaggatcaaattacttagttacagctggtatgaaacgttttgagaaatcagtttcagtaaacttgaaatataactgagtcttctttcagtaggagaccttgacttcaaattgagaggacttccaggatactgtgggagttatattttttttatgggaaaactatttctatcatgaagtaatgcattgttgttagacctaatattgtgagtgtctaatacttatgtttatataattgcacttccagtaaagttattgtggcaaccatttcacattgtcttttaaagaacatgcccttggcatagaaatactatttagttttaatggtagcctcattgatgttcagtacgtttttcactattaagacacttgacaaacaatgagatatttactttttatcccttgtaggcagaaagctgtaaaaataaacttcagctttcagtgttc

(19) United States
(12) Patent Application Publication
(10) Pub. No.: US 2009/0286689 A1
(43) Pub. Date: Nov. 19, 2009

(54) METHODS FOR ANALYZING DIFFERENTIAL GENE EXPRESSION ASSOCIATED WITH MYELOPROLIFERATIVE DISORDERS (MPD) CANCER DISEASE

(75) Inventors: Daniel BIRNBAUM, Marseille (FR); Helene Lelievre, Marseille (FR); Nathalie Cervera, Bonnieux (FR)

Correspondence Address: HARNESS, DICKEY & PIERCE, P.L.C., P.O. BOX 828, BLOOMFIELD HILLS, MI, 48303, US

(73) Assignee: INSERM (Institut National de la Sante et de la Recherche Medicale), Paris (FR)

(21) Appl. No.: 11/953,899

(22) Filed: Dec. 11, 2007

Related U.S. Application Data
(60) Provisional application No. 60/874,357, filed on Dec. 12, 2006.

(51) Int. Cl. C40B 30/00; C12Q 1/68; C40B 40/06

(52) U.S. Cl. 506/7; 435/6; 506/16

(57) ABSTRACT

The present application relates to gene analysis and, in particular, to gene expression profiling for identifying molecular signature of cancer disease, in particular the G1 phase of the cell cycle, such as myeloproliferative disorders (MPD) or breast cancer and studying cancer.

More particularly, the application is directed to a method for analyzing differential gene expression associated with cancer disease, in particular the G1 phase of the cell cycle, such as myeloproliferative disorders (MPD) or breast cancer comprising detection of upregulation and/or downregulation of a pool of polynucleotide sequences in a cell or tissue sample, said pool corresponding to all or part the polynucleotide sequences, subsequences or complements thereof, of the genes listed in Tables 1, 2 and 3.

### PRIORITY

This Application claims priority to U.S. Provisional Application No. 60/874,357 filed on 12 Dec. 2006; the entire contents of which are hereby incorporated herein by reference.

### TECHNICAL FIELD

This disclosure relates to gene analysis and, in particular, to gene expression profiling for identifying molecular signature of cancer disease, in particular the G1 phase of the cell cycle, such as myeloproliferative disorders (MPD) or breast cancer and studying cancer.

### BACKGROUND

Myeloproliferative disorders (MPD) are clonal proliferative diseases of the hematopoietic stem cells. After an initial phase they may progress to an acute syndrome. V617F mutation of the JAK2 kinase are found in polycythemia vera, essential thrombocythemia, and idiopathic myelofibrosis. BCR-ABL fusion occurs in chronic myeloid leukemia, and a variety of fusions involving PDGF and FGF receptors are found in other MPDs. Fusion kinases that result from a translocation are made of a constitutively activated kinase domain and an N-terminal region encoded by a partner gene. The activated kinase triggers sustained proliferation and survival of the hematopoietic cells but maturation is not affected.

Because of the side effects and risk of cancer therapy as well as resistance of certain patient to the treatment with certain drug, it would be desirable to offer new means to monitor cancer treatment and to be able to identify patients in need of such treatment.

### SUMMARY

We provide methods for analyzing differential gene expression associated with cancer disease, more particularly, the G1 phase of the cell cycle such as myeloproliferative disorders (MPD) or breast cancer, comprising detecting the upregulation and/or downregulation of a pool of polynucleotide sequences in a cell or tissue sample, the pool corresponding to all or part the polynucleotide sequences, subsequences or complement thereof, of the genes listed in Tables 1, 2 and 3.

More particularly, the methods are carried out on a tumor cell or tissue sample. It may be any sample that may be taken from a patient, such as for example serum, plasma, urine or a biopsy sample.

Upregulation and/or downregulation of a pool of polynucleotide sequences according to the method of the present invention identify a molecular signature of activated MPD kinase.

Table 2 displays 188 upregulated genes/EST and table 3 displays 48 genes/EST downregulated in activated kinase-expressing cells.

Table 1 represents the most significant (p-value inferior at 2·10⁻²) and most often represented (including at least 3 genes) biological processes. Many of the upregulated genes encode nucleolar proteins involved in “ribosome biogenesis” (GO:0007046, 6 genes, p=4.28·10⁻¹¹), “rRNA processing” (GO:0006364; 7 genes, p=3.07·10⁻¹¹), and “protein biosynthesis” (GO:0006412, 9 genes, p=3.60·10^{-0.5}).

Tables 1, 2 and 3 indicate the name of the gene (gene Symbol). We define the nucleotide sequences by the name of the gene or fragments thereof. Each polynucleotide sequence in Tables 2 and 3 may be considered as a marker of the corresponding gene. Each marker corresponds to a gene in the human genome, i.e., such marker is identifiable as all or a portion of a gene. Any RNA transcribed from a marker gene (mRNAs), any cDNA or cRNA produced therefrom, and any nucleic acid derived therefrom, such as synthetic nucleic acid having a sequence derived from the gene corresponding to the marker gene are also encompassed by the present invention.

A “pool of polynucleotide sequences” may comprise one or more sequences, preferably 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 350, 400, 450, 500 sequences.

The number of genes may vary in the range of from 1 to the total number of genes described in Tables 1, 2 or 3, for example, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 188 genes, depending of the number of genes in said tables.

The pool of polynucleotide sequences may comprise the polynucleotide sequences, subsequences or complement thereof, of the genes listed in Tables 2. More particularly, the methods relate to upregulated genes encoding nucleolar proteins (CIRH1A, LARP1, NOL1, NOL11, NOL5, NOL5A, NOLA1, NOLA2, NOLC1, MKI67IP, SFRS2, SURF6), ribosomal proteins (RPL3, RPL12, RPL41, RPS9, RRS1), small nuclear ribonucleoproteins and interactors (U3/MPHOSPH10, LSM2, RNU22, RNU3IP2), components of RNA polymerase I (POLR1A, POLR1B), II (POLR2H, TAF9) and III (POLR3E, POLR3H), DEAD-box (DDX18, DDX56) and WD repeat (WDR4, WDR43, WDR74, WDR77, GRWD1, PWP1) proteins, eukaryotic initiation and elongation factors (EIF1A, EIF3S1, EIF3S4, EEF1E1), and components of the exosome (EXOSC1, EXOSC2, EXOSC6).

The genes also relate to upregulated genes encoding proteins of the NOL5A-associated preribosomal ribonucleoprotein complex involved in pre-rRNA processing: NOL5A, PPAN, NOLC1, and BXDC2. The gene encoding EBNA1BP2 was upregulated; it encodes a protein that binds to nucleolar FGF3 and is regularly upregulated in tumors.

The genes further relate to the most upregulated sequence GAS5, a non-protein-coding multiple small nucleolar RNA (snoRNA).

Other significant processed genes includes “protein folding” (4 genes), “ubiquitin-dependent protein catabolism” (3 genes), “nuclear mRNA splicing, via spliceosome” (3 genes), and “regulation of cell cycle” (3 genes).

A second major category of upregulated genes encode CCND2 (cyclin D2) and CDC25A, two major regulators needed for G1 progression.

Moreover, the list of upregulated genes included MYC. Many genes upregulated by MYC and NMYC oncogenes were also upregulated in our experiments, including CCND2, CDC25A and others (DDX18, EBNA1BP2, EEF1E1, MAT2A, MKI67IP, NOL5A, NOLA1, PHB, SFRS2, SHMT1, SLC16A1, SURF6, SRM, RPL3, RPL12, RPL41, RPS9 and RRS1).

The pool of polynucleotide sequences may include all or part the polynucleotide sequences, subsequences or complement thereof, of the genes listed in Tables 3. More particularly, the pool relates to downregulated genes encoding proteins with known or potential inhibitory function such as PIAS3, an inhibitor of STAT3, one of the main substrates of MPD kinases, and regulator of CDC25A, Erbin, and PLZF/ZBTB16, a MYC repressor.

The detection of over or under expression of polynucleotide sequences may be carried out by FISH or IHC. The detection may be performed on DNA microarrays. The level of the RNA transcripts can be measured by any available technique such as quantitative PCR.

We further provide a polynucleotide library useful for the molecular characterization of a cancer comprising or corresponding to a pool of polynucleotide sequences either upregulated or downregulated in tissue, said pool corresponding to all or part of the polynucleotide sequences selected as defined above.

The polynucleotide library may be immobilized on a solid support, for example selected from the group comprising nylon membrane, nitrocellulose membrane, glass slide, glass beads, membranes on glass support or silicon chip, plastic support.

We further provides a method for analysing differential gene expression associated with cancer disease, comprising:
a) obtaining a polynucleotide sample from a patient, and
b) reacting the polynucleotide sample obtained in step (a) with a polynucleotide library as defined above, and
c) detecting the reaction product of step (b).

The polynucleotide sample may be labelled before reaction step (b). The label may be selected from the group consisting of at least one of radioactive, calorimetric, enzymatic, molecular amplification, bioluminescent or fluorescent labels. Preferably, the label is calorimetric, e.g., biotin or digoxygenin.

The method may further comprise:
a) obtaining a control polynucleotide sample,
b) reacting said control sample with the polynucleotide library, for example by hybridising the polynucleotide sample with the polynucleotide library,
c) detecting a control sample reaction product, and
d) comparing the amount of the polynucleotide sample reaction product to the amount of the control sample reaction product.

The polynucleotide sample may be cDNA, RNA or mRNA. The mRNA may be isolated from the polynucleotide sample and cDNA may be obtained by reverse transcription of said mRNA.

We further provide a method for determining the nature of a cancer from a cancer cell or tissue comprising the implementation of the method on nucleic acids from cells or tissues from a patient.

More particularly, the method is useful for profiling samples of various human MPDs. It may help define the initial steps of oncogenesis, for which G1 activation may be important. Cell quiescence may be defined by the absence of protein synthesis, whereas proliferation starts with nucleolar activity, ribosome biogenesis and rRNA processing. Second, MPDs can benefit from treatment targeting not only the activated kinases but also the PI3K/AKT/TOR signaling pathway and the G1 phase of the cell cycle, in synergy with anti-kinase drugs and/or in case of resistance. G1 targeting is frequent in many types of cancer. The signature can be used in transcriptome studies of any type of cancer to identify G1 activation, classify tumors, and use the appropriate drugs.

Therefore, we further provide a method of prognosis or diagnostic or prediction of tumours susceptible to a molecule acting on the G1 phase of the cell cycle such as rapamycine and its derivatives, and therefore we provide a method for monitoring the treatment of a patient with a cancer comprising the implementation of the method of analysis disclosed hereabove on nucleic acids from a patient to identify the corresponding signature.

We also provide a method of selection of patient susceptible to be treated with molecule acting on the G1 phase of the cell cycle such as rapamycine or its derivative, comprising the implementation of the method hereabove on nucleic acids from a patient in order to identify the corresponding signature.

This signature provides tools for classifying basal tumors for which the high proliferation results from the PI3K/TOR pathway. Therefore, we provide methods for determining whether a tumor has an activated proliferation according to PI3K/TOR and is sensitive to chemotherapy.

### BRIEF DESCRIPTION OF THE DRAWING

FIG. 1 shows gene expression profiling of Ba/F3 cell samples identifies a molecular signature of activated MPD kinases. A. Hierarchical clustering of 15 samples using the 294 genes identified as discriminator between Ba/F3 cells transfected by activated kinases (9 samples: BCR-ABL, 2 BCR-FGFR1, 2 CEP1-FGFR1, 2 FOP-FGFR1 and 2 V617F JAK2) to that of parental Ba/F3 cells (4 samples) and Ba/F3 expressing a kinase defective mutant of FOP-FGFR1 (2 samples). Each row represents a gene, each column a sample. The log₂-transformed expression level of each gene in a single sample is relative to its median abundance across all samples and is depicted according to the colour scale shown at the bottom. Red and green indicate expression levels respectively above and below the median. The magnitude of deviation from the median is represented by the color saturation. The dendrogram of samples (above matrix) represents overall similarities in gene expression profiles and is zoomed in B. Branches of the dendrograms are color-coded as follows: red for fusion kinase-expressing Ba/F3 cells and green for control cells. Some genes included in the signature (framed in red for the upregulated genes and in green for the down-regulated genes) are noted to the right of the data matrix and referenced by their abbreviation as used in EntrezGene. A discriminating score (DS) was calculated for each gene. DS=(M1−M2)/(S1+S2) where M1 and S1 respectively represent mean and SD of expression levels of the gene in subgroup 1, and M2 and S2 in subgroup 2. Confidence levels were estimated by 100 random permutations of samples. A “leave-one-out” procedure estimated the accuracy of prediction of the signatures and the validity of our supervised analysis. B. Top, dendrogram of samples. Down, correlation between the molecular grouping based on the combined expression of the 294 genes and the status of samples. C. Western blot analysis of cyclin D2 in Ba/F3 expressing activated MPD kinases. NP40-extracted proteins were separated by gel electrophoresis (SDS-PAGE), transferred onto membrane (Hybond-C Extra, GE Healthcare UK, Buckinghamshire, UK), and probed with rabbit polyclonal anti-cyclin D2 (M-20, Santa Cruz Biotechnology, Santa Cruz, Calif.). Cyclin D2 (top) is upregulated in Ba/F3 cells expressing MPD kinases compared to control Ba/F3 cells (untransfected, FOP-FGFR1 KD, MIGR and JAK2 WT). Total cell lysates were probed with mouse monoclonal anti-α-tubulin (B-5-1-2, Sigma-Aldrich, Saint-Quentin Fallavier, France) to compare the amount of protein in the lysates (bottom).

FIG. 2 provides the murine polynucleotide sequences from Ba/F3 cell line of the genes/EST listed in tables 1, 2 and 3 where the genes/EST are identified by their names and access numbers (Prob set).

### DETAILED DESCRIPTION

Myeloproliferative disorders (MPD) are clonal hematopoietic diseases characterized by the proliferation and expansion of one or several myeloid cell lineages in the bone marrow. During the chronic phase, the cells follow their normal differentiation pathway and become mature blood cells. During a second phase, an acute syndrome may occur. The conventional classification separates MPDs in clinical entities. These include chronic myeloid leukemia (CML), chronic neutrophilic leukemia, chronic eosinophilic leukemia, polycythemia vera (PV), essential thrombocythemia (ET) and idiopathic myelofibrosis (IMF). MPDs with different syndromes and molecular abnormalities are grouped in non-classical MPDs. Systemic mastocytosis is not classically included in MPDs but is a related disease.

We studied the gene expression profiles of murine Ba/F3 cells transfected by various oncogenic MPD kinases by using whole-genome Affymetrix 430 2.0 mouse oligonucleotide microarrays (www.Affymetrix.com). Ba/F3 cells were grown in RPMI medium supplemented with 10% fetal calf serum (FCS) and IL3. Expression of an MPD kinase bypasses the IL3 dependence of Ba/F3 cells. RNA was extracted by using Trizol (Trizol Reagent, Invitrogen Life Technologies, Carlsbad, Calif.) from frozen pellets of: i)—Ba/F3 cells, ii)—Ba/F3 transfected with different pCDNA expression vectors expressing a mutant, kinase-defective FOP-FGFR1 KD or an oncogenic MPD kinase (BCR-ABL, FOP-FGFR1, CEP1-FGFR1, BCR-FGFR1), and iii)—Ba/F3 transfected by the MIGR vector, either empty (MIGR) or with JAK2 (JAK2 WT, mutated V617F JAK2 or mutated V617F IND JAK2 i.e. able to grow independently of IL3). Before RNA extraction, cells were starved for 7 hours in RPMI plus 0.5% FCS. RNA integrity was controlled by microanalysis (Agilent Bioanalyzer, Palo Alto, Calif.). Preparation of cRNA, hybridizations, washes, detection and quantification were done as recommended by the supplier (Affymetrix). Data were analyzed by the RMA (Robust Multichip Average) method in R using Bioconductor and associated package. Before analysis, a filtering process removed from the dataset the genes with low and poorly measured expression as defined by an expression value inferior to 100 units in all samples, retaining 17.885 genes/ESTs. For paired samples, RNA was prepared independently from different cultures of cells. The correlation between paired samples ranged between 0.97 and 0.98.

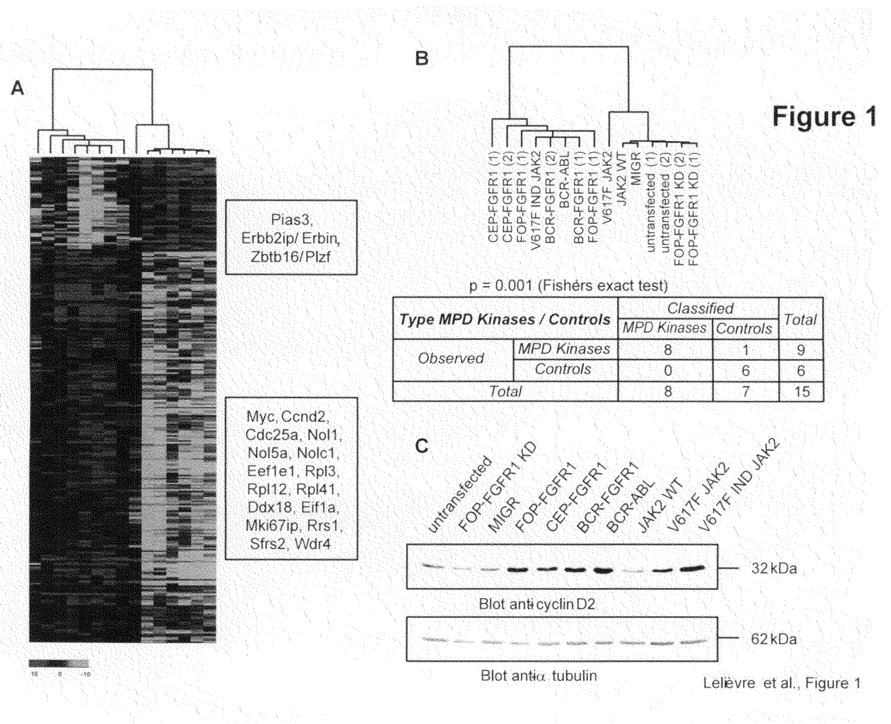

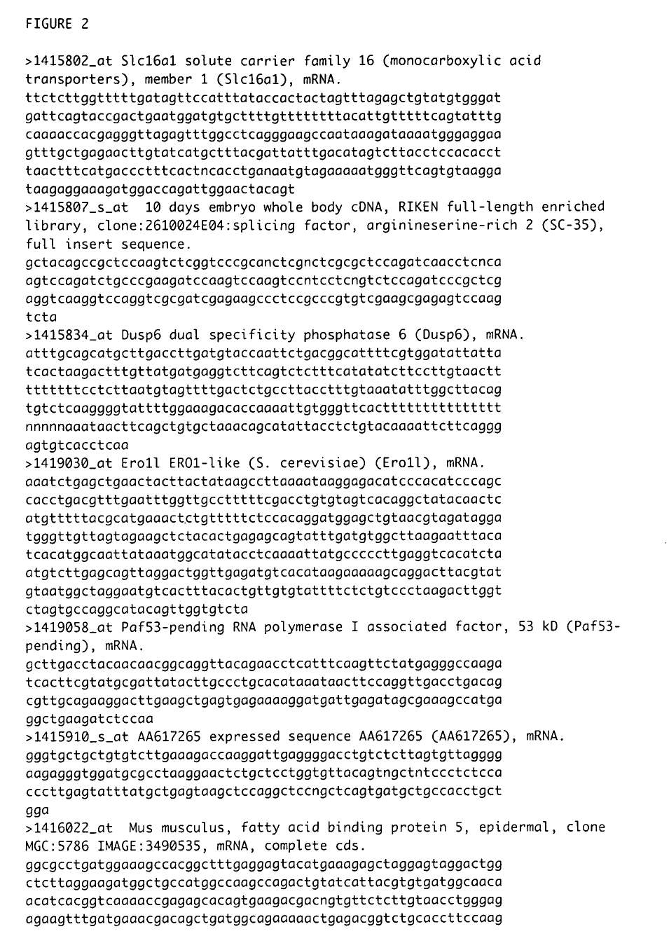

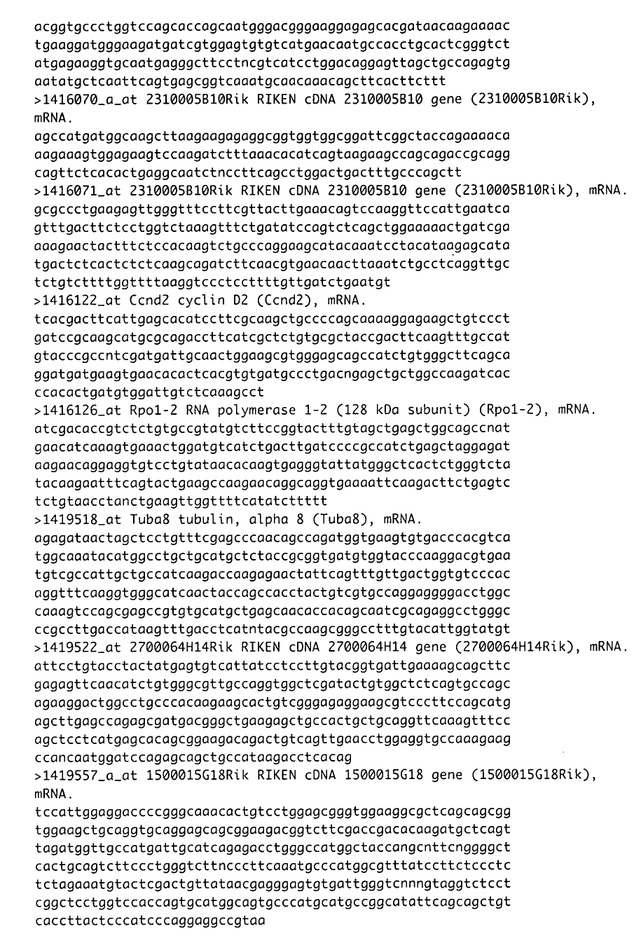

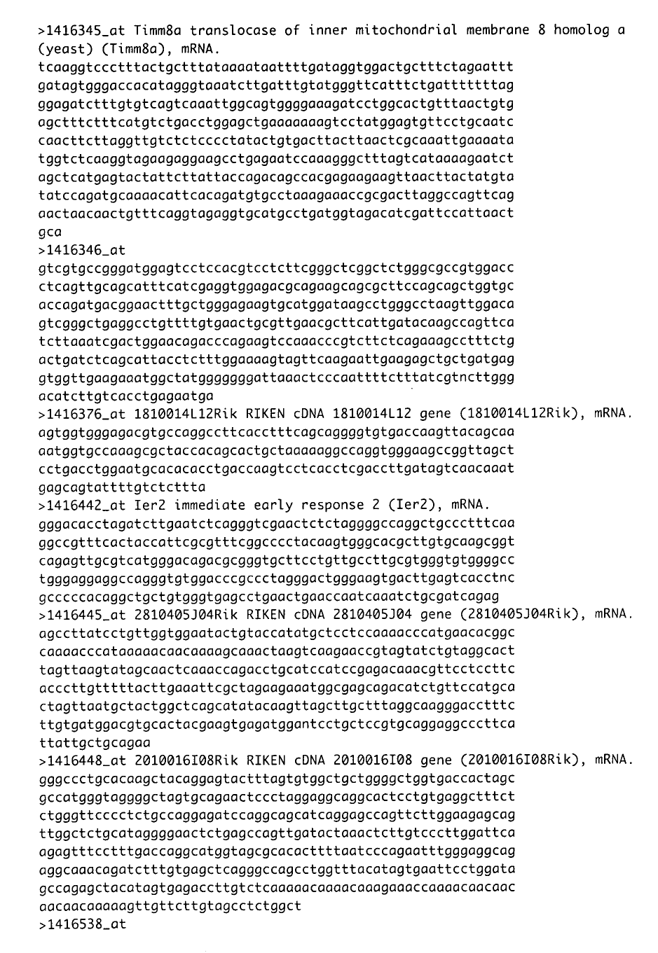

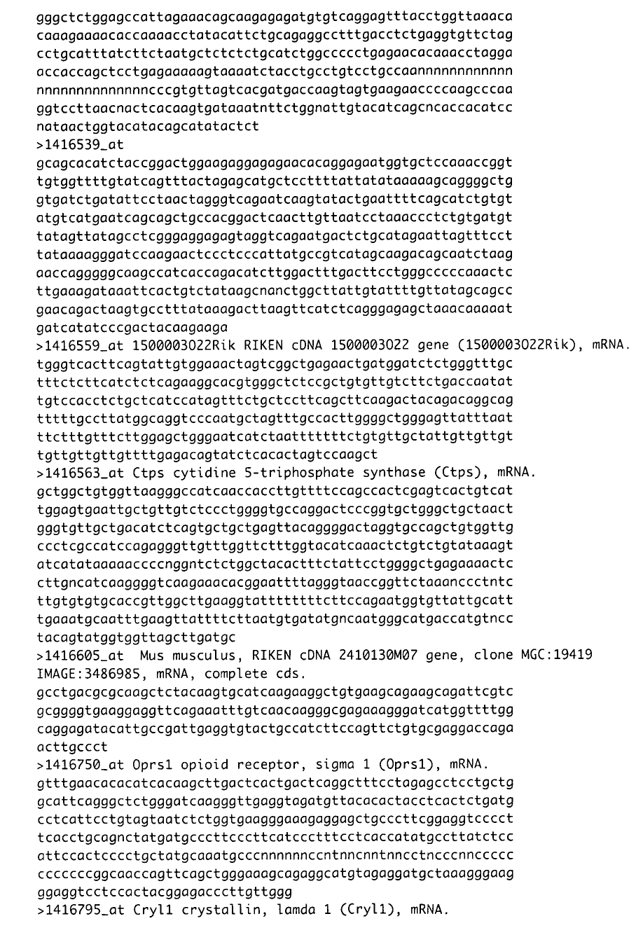

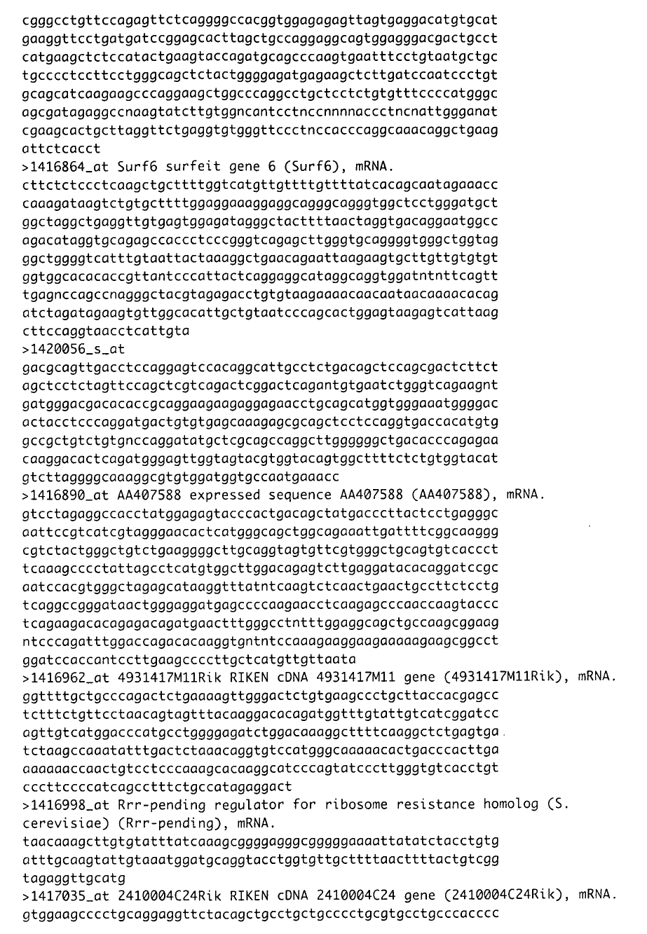

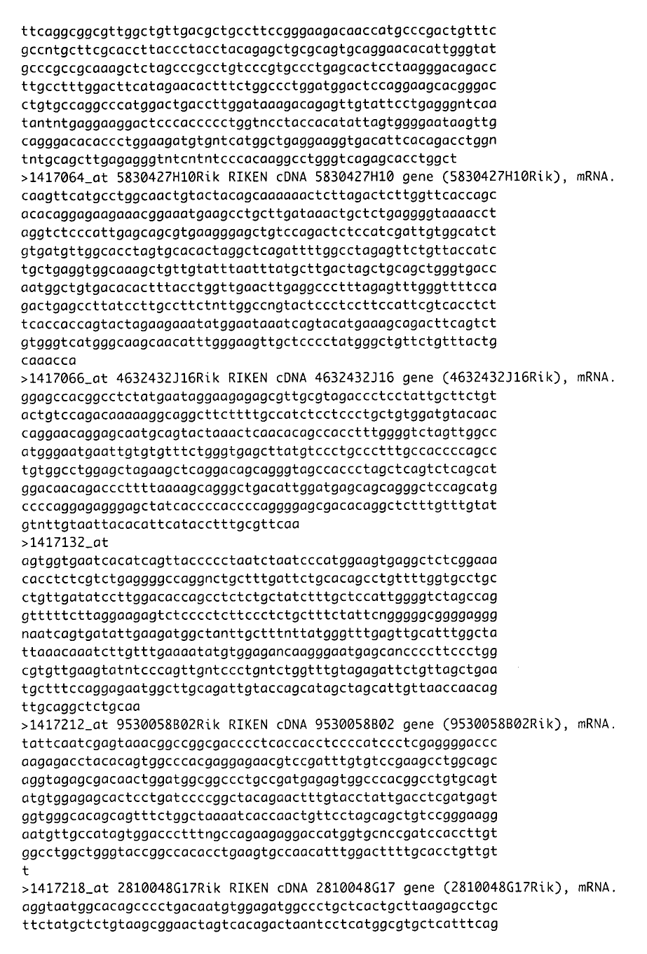

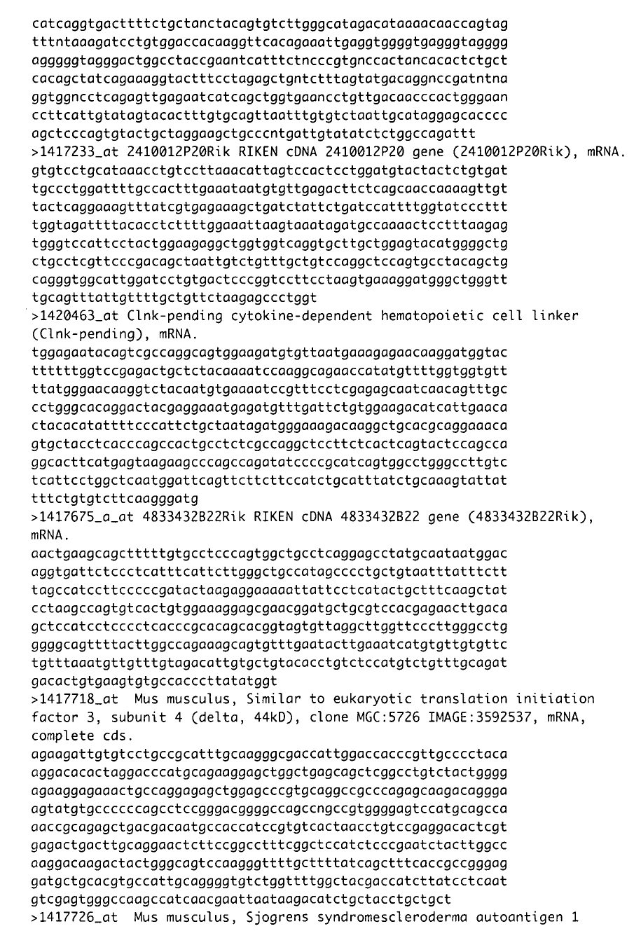

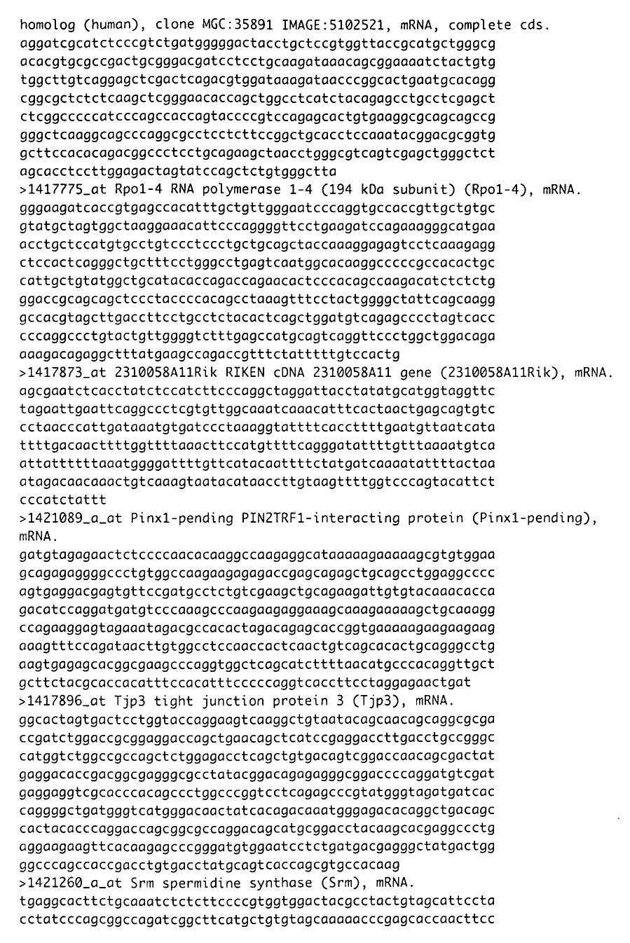

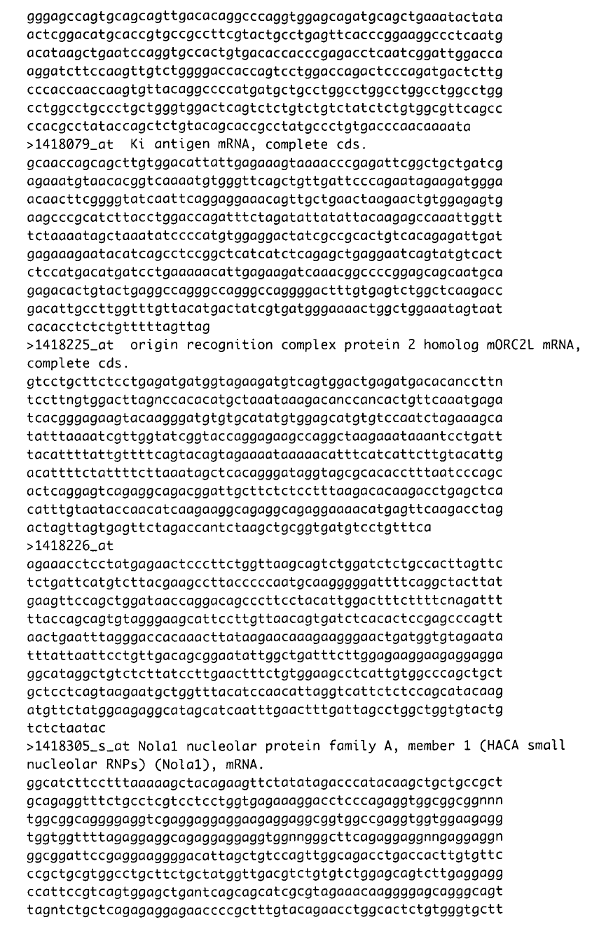

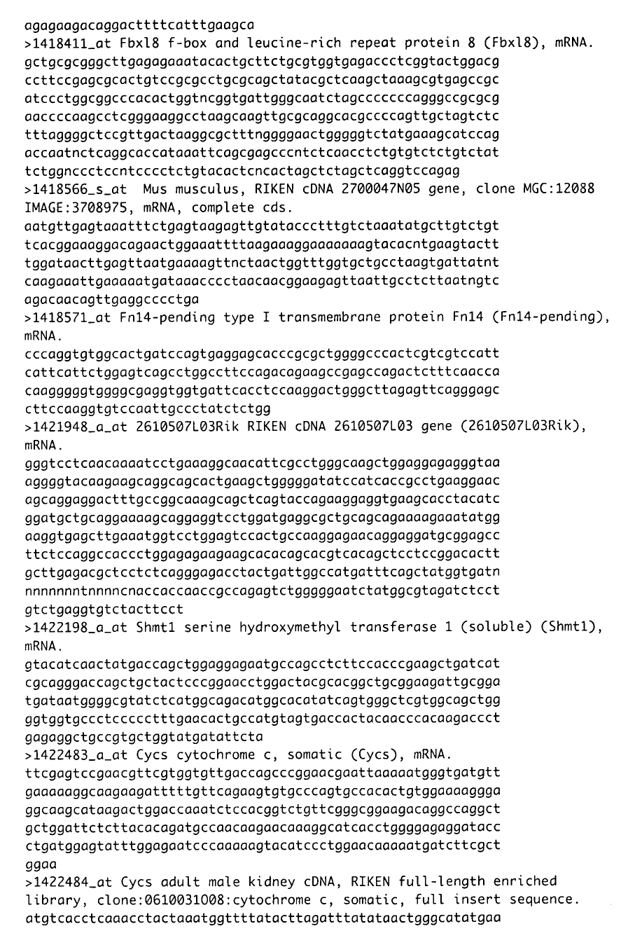

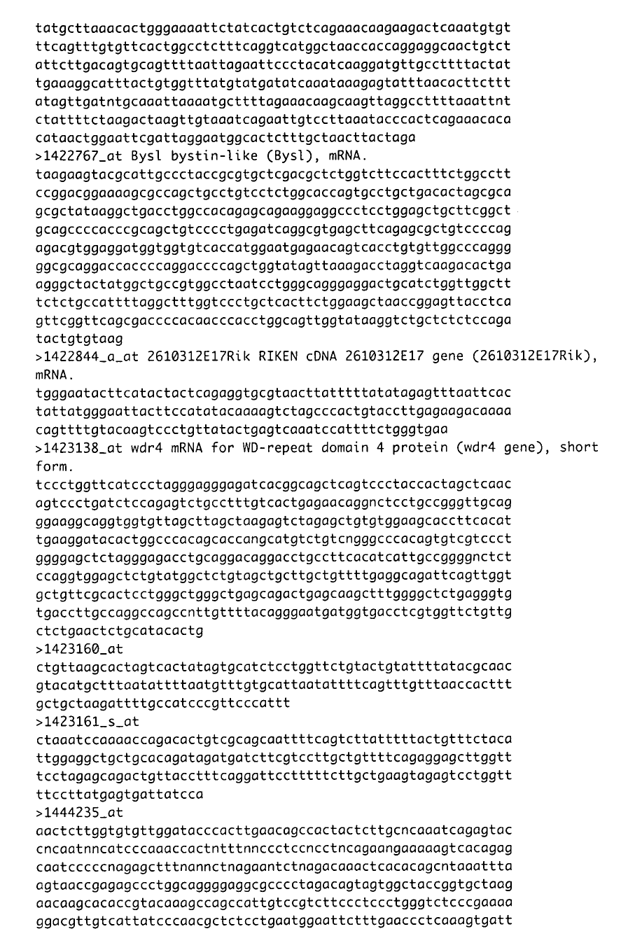

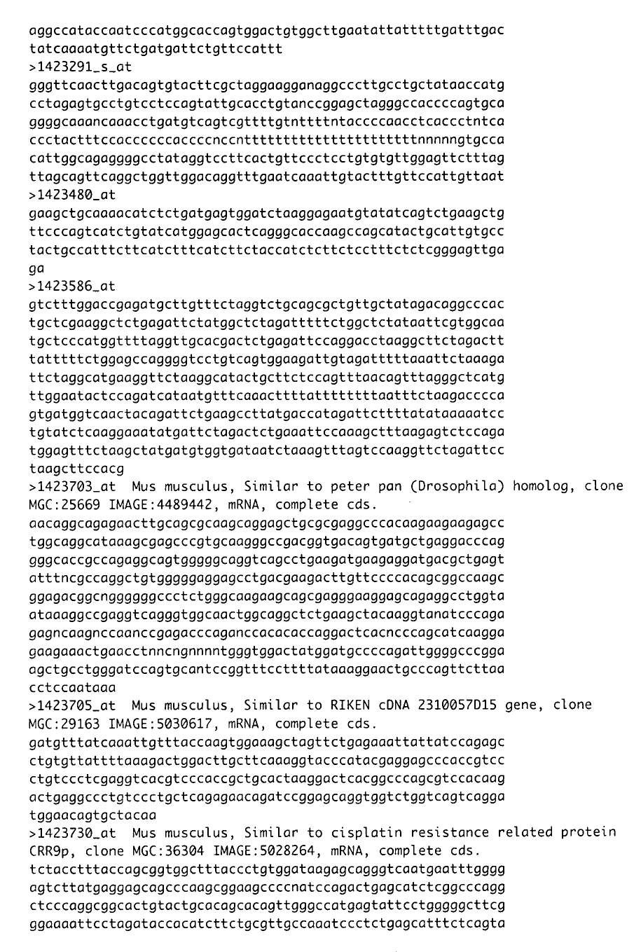

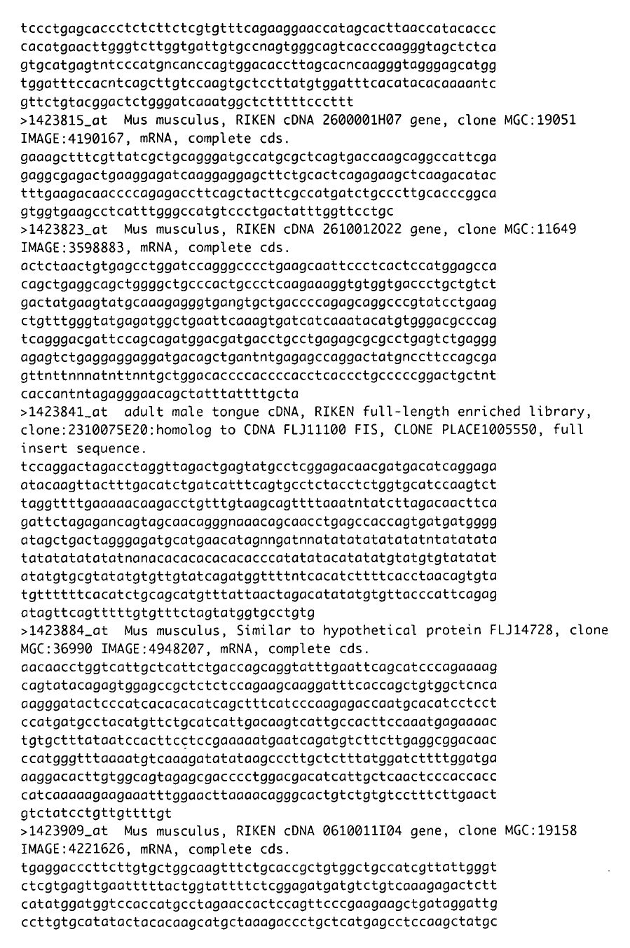

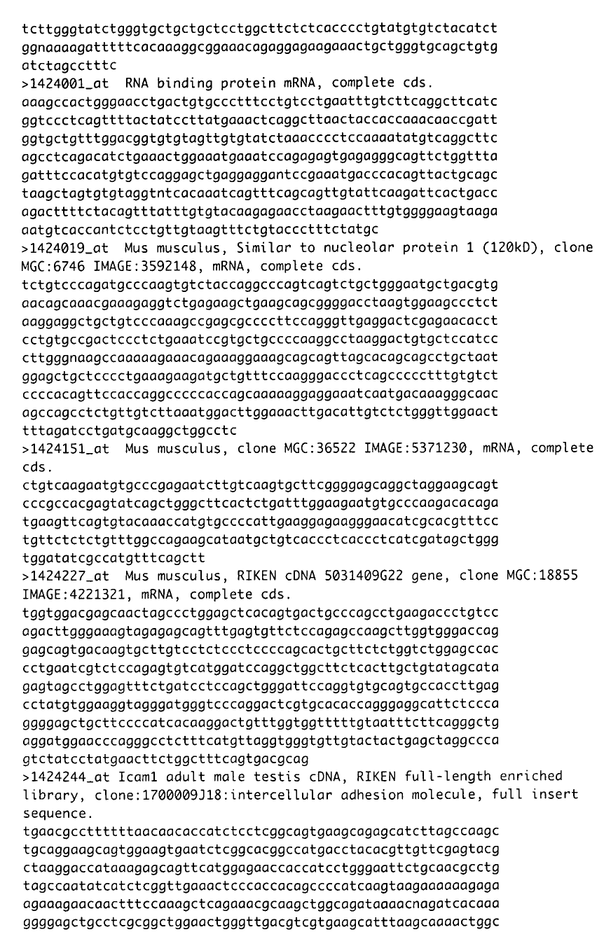

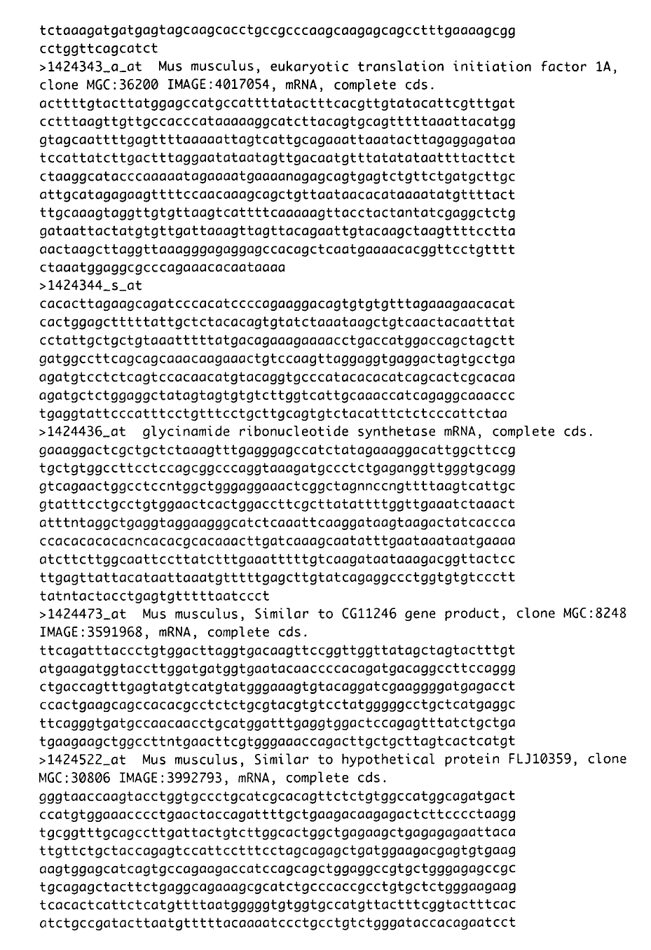

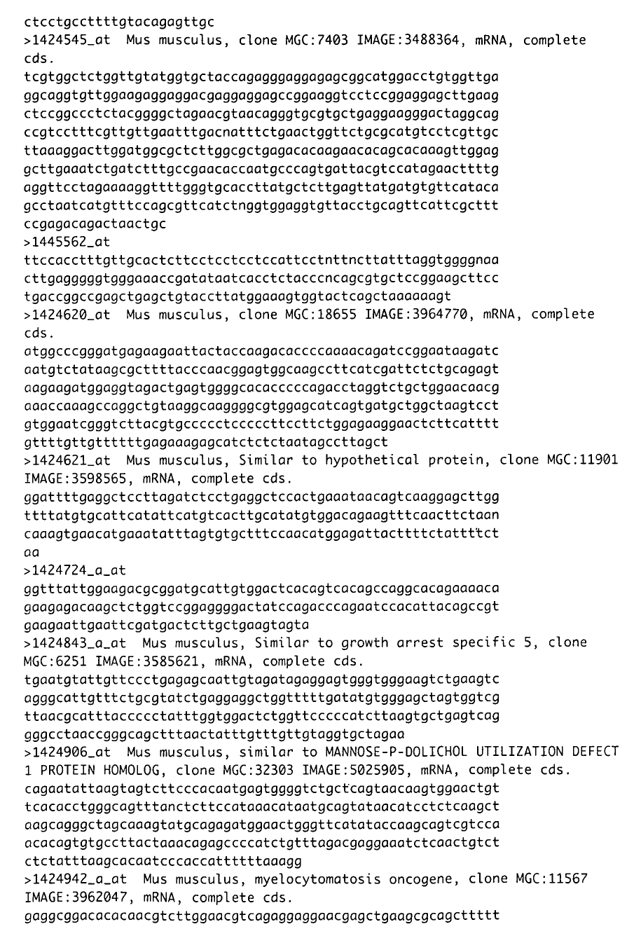

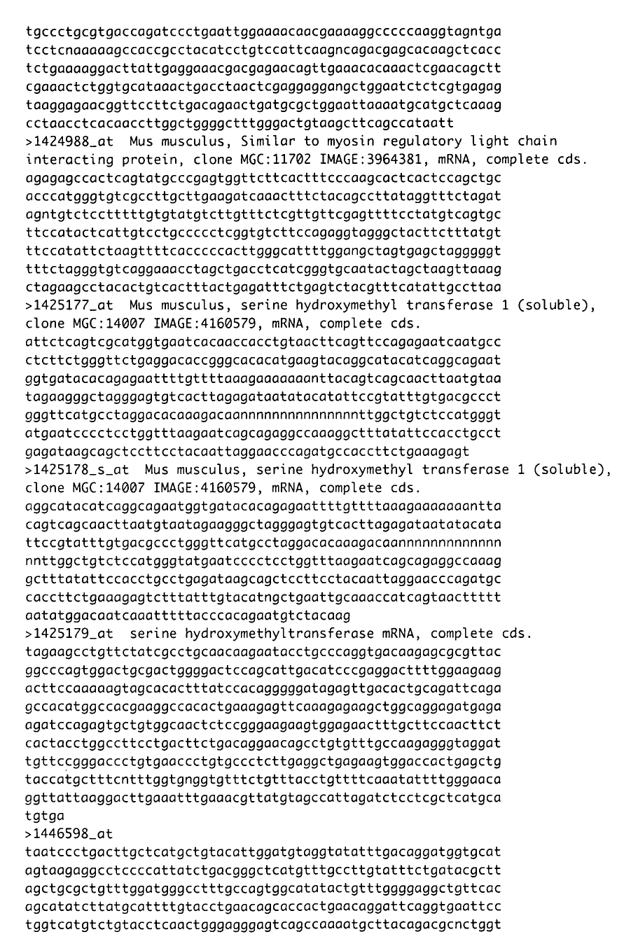

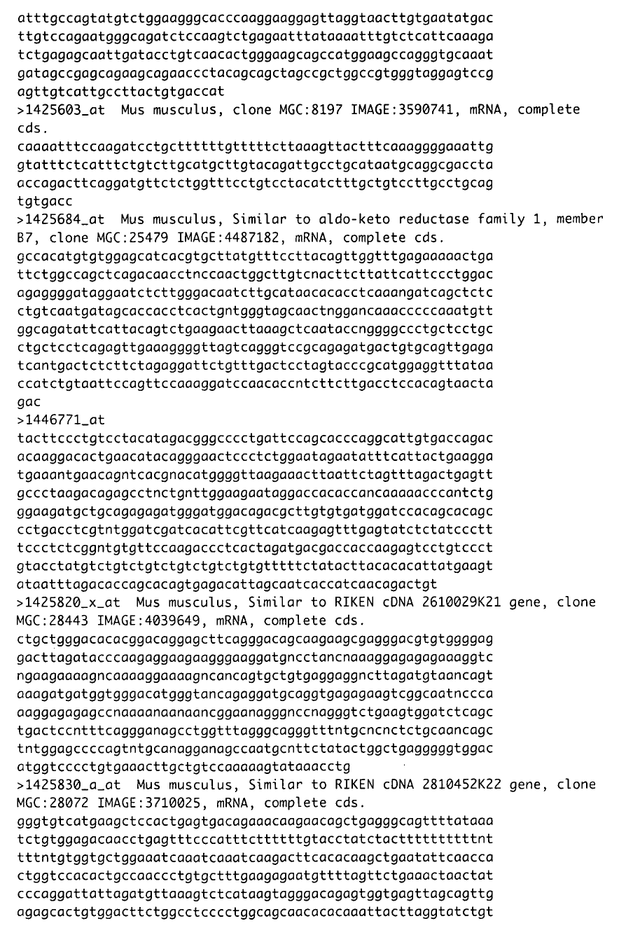

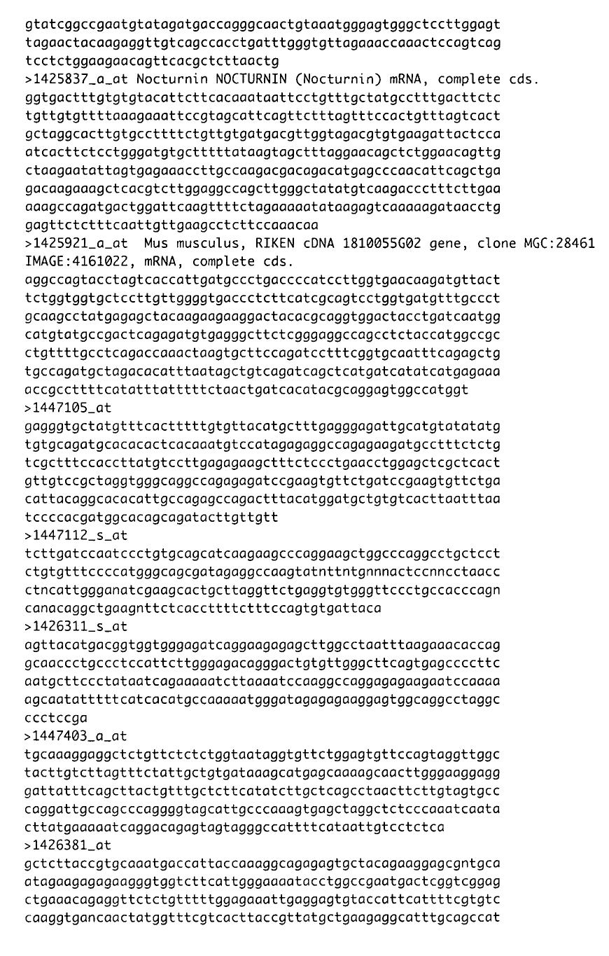

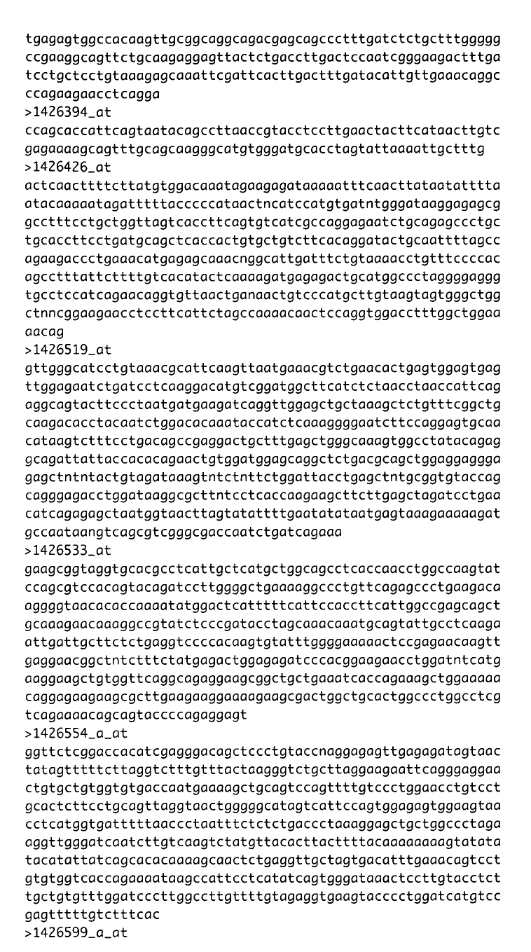

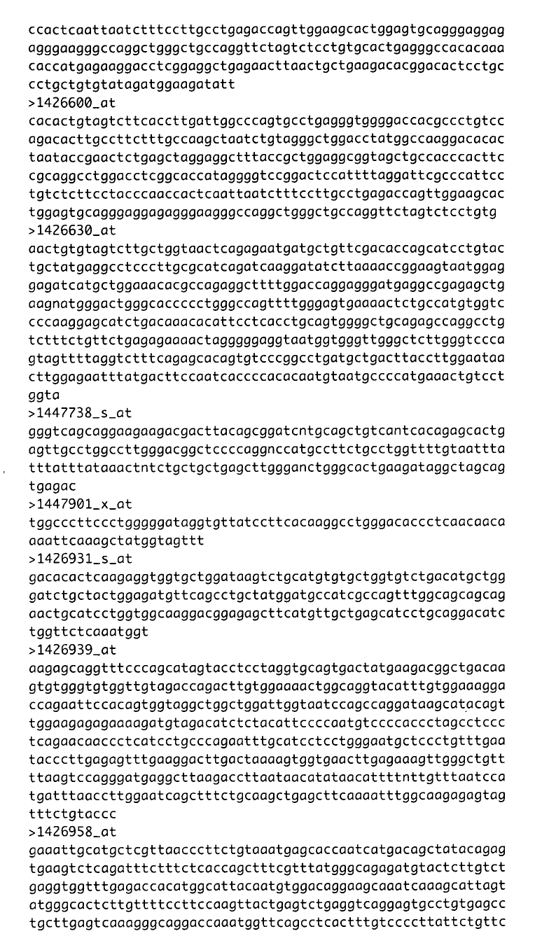

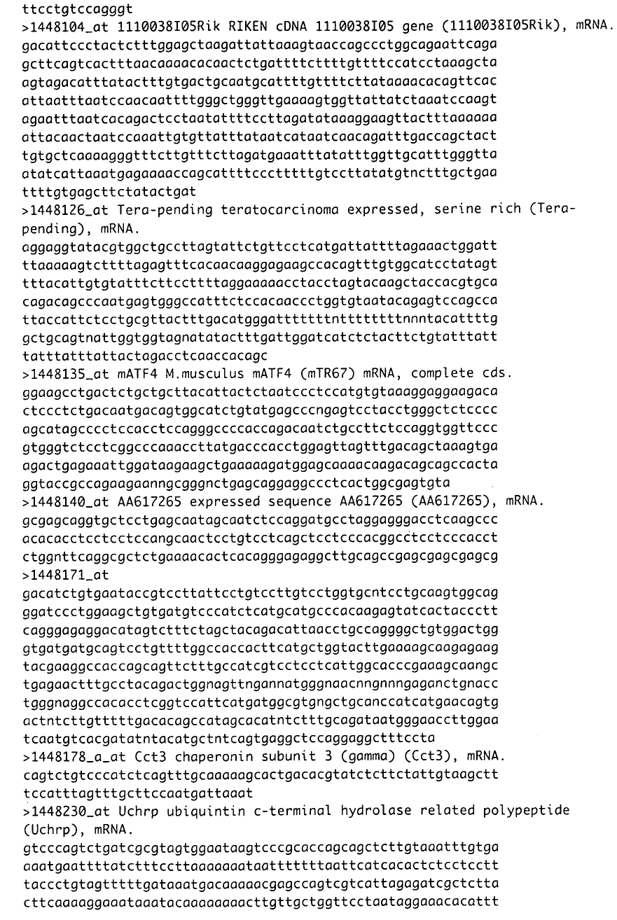

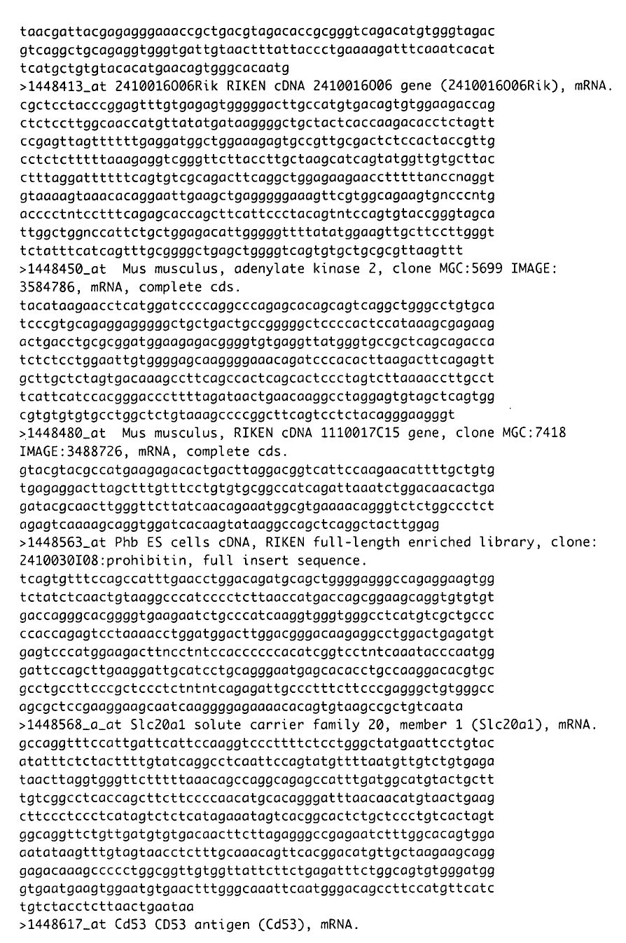

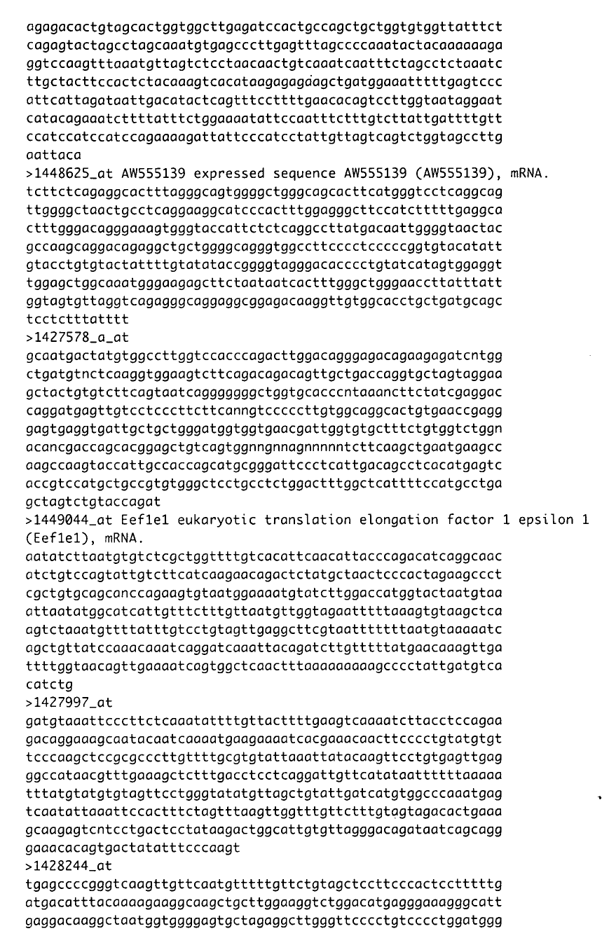

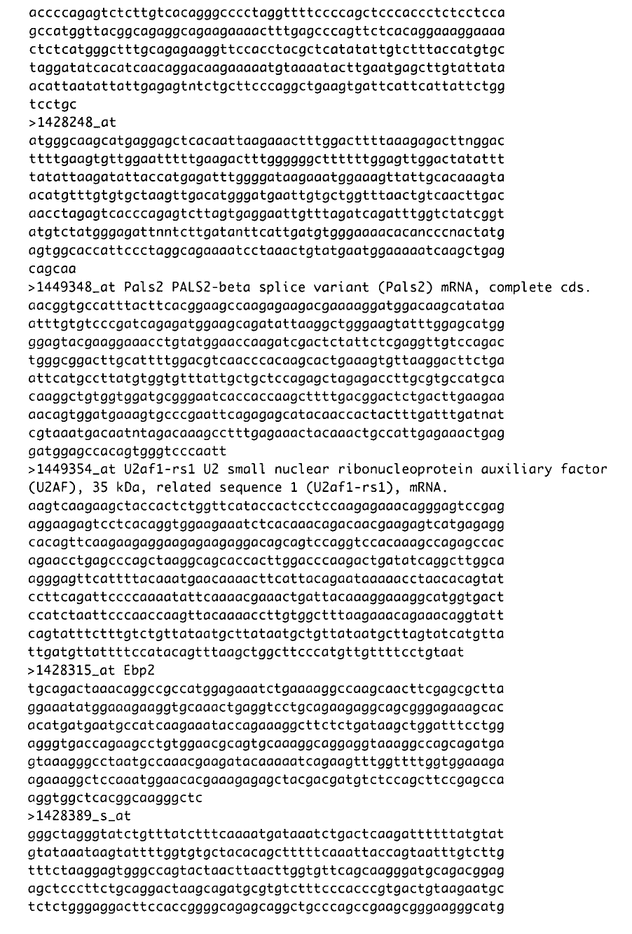

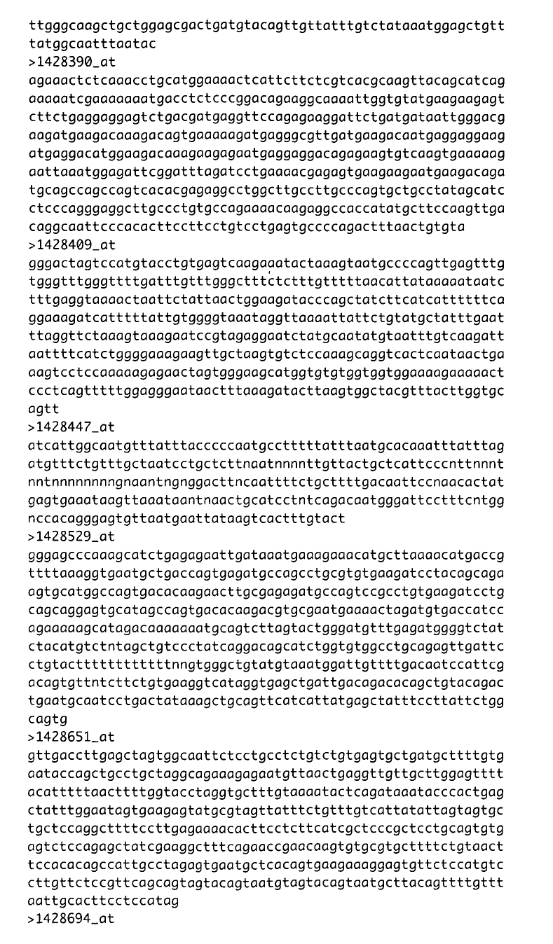

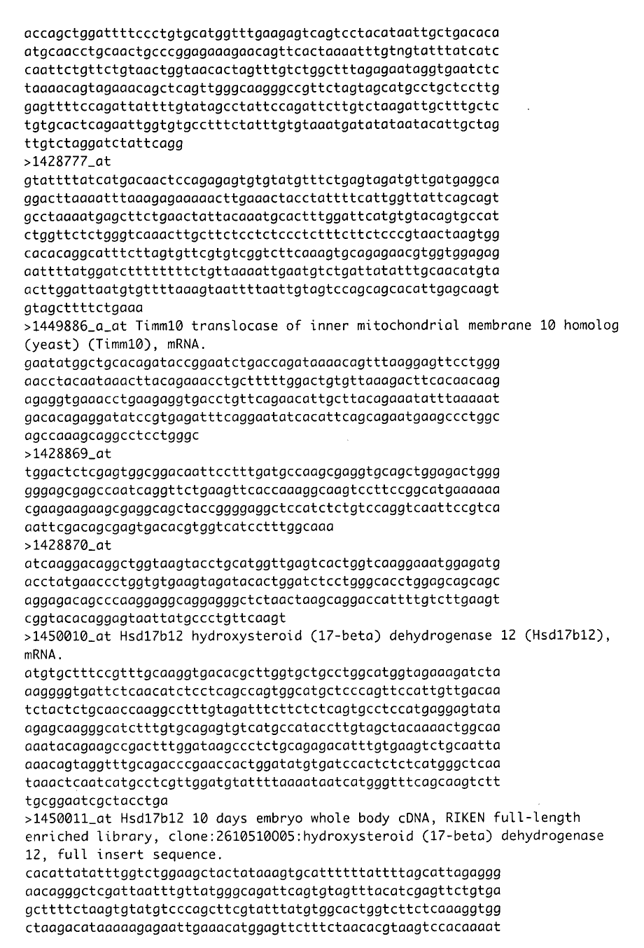

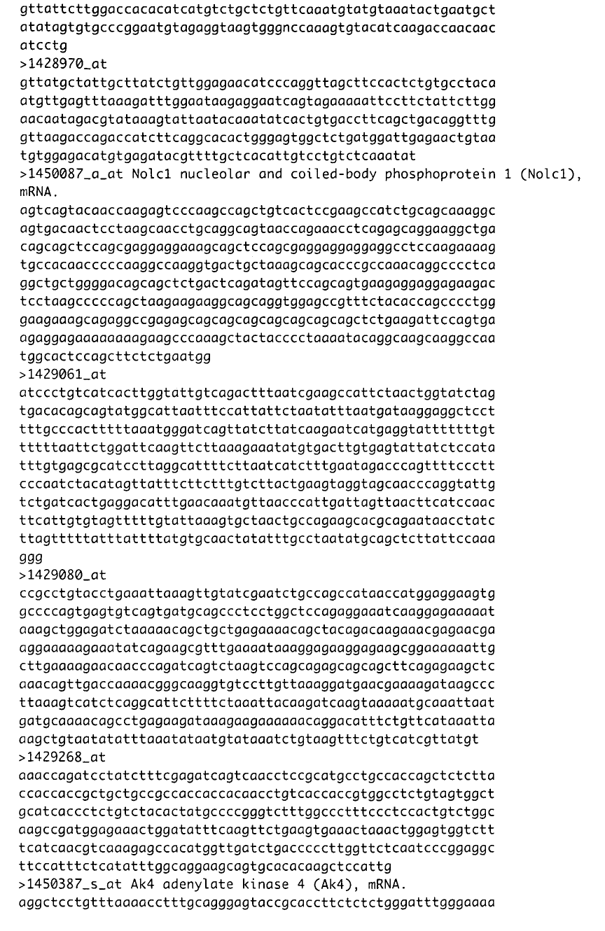

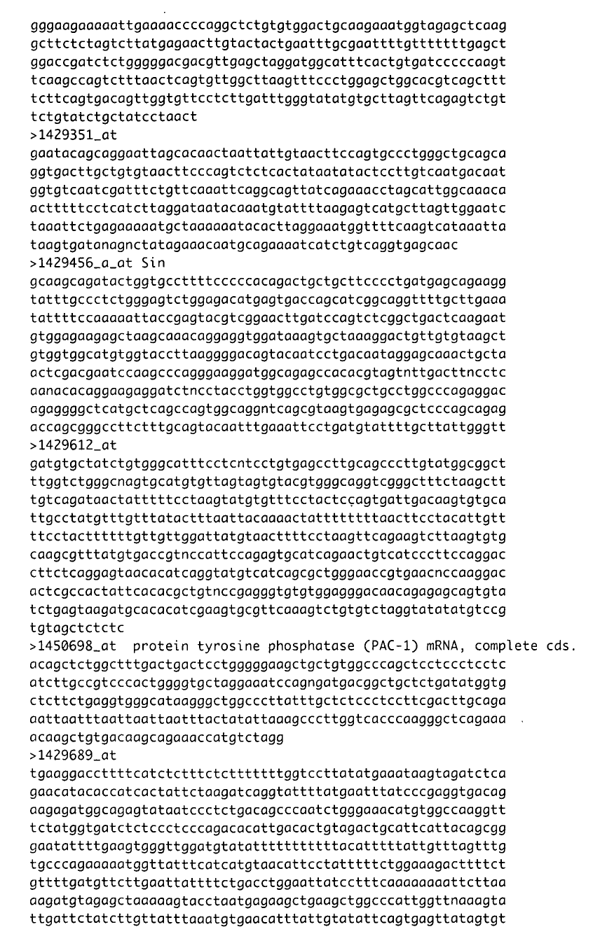

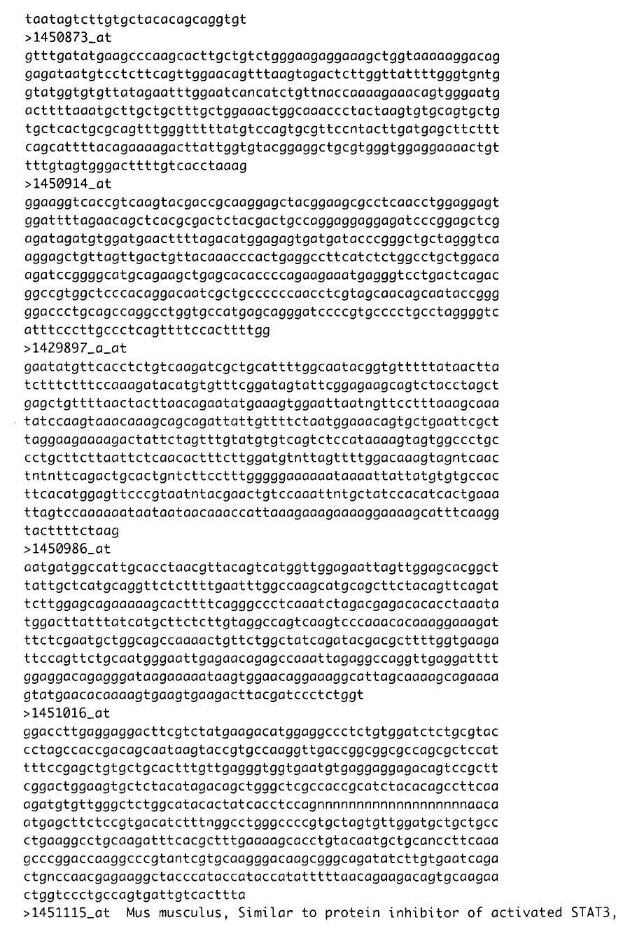

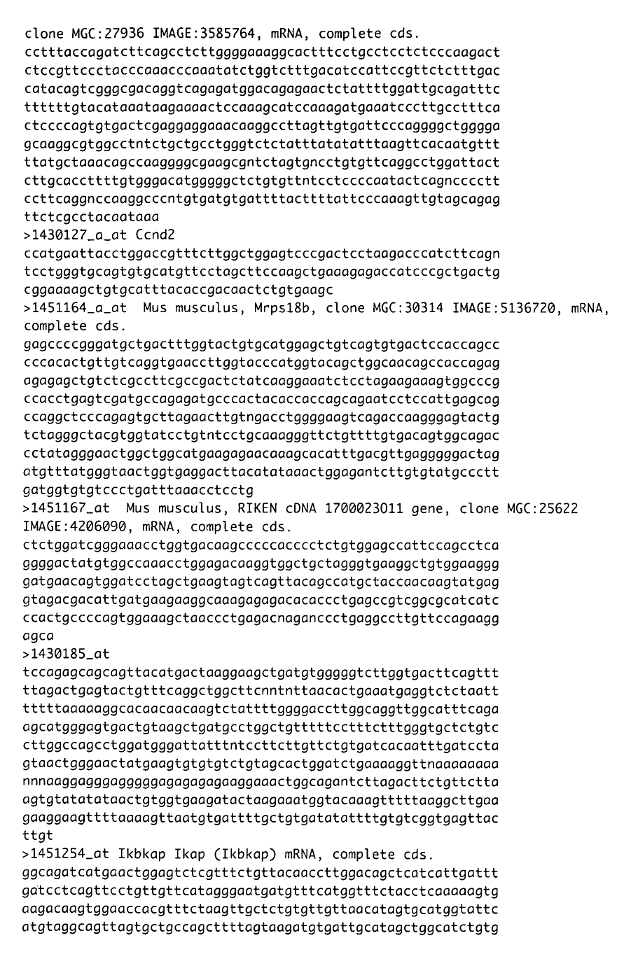

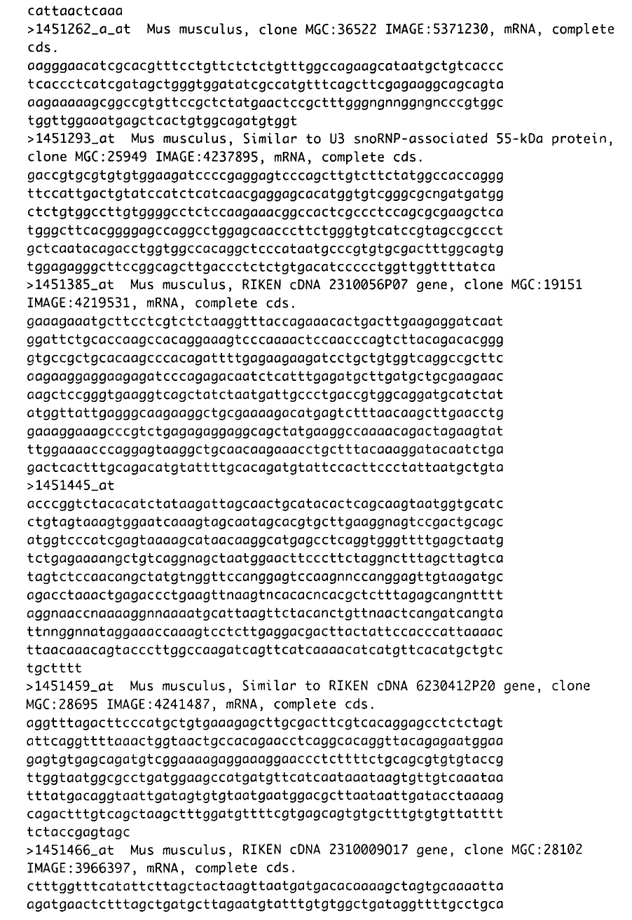

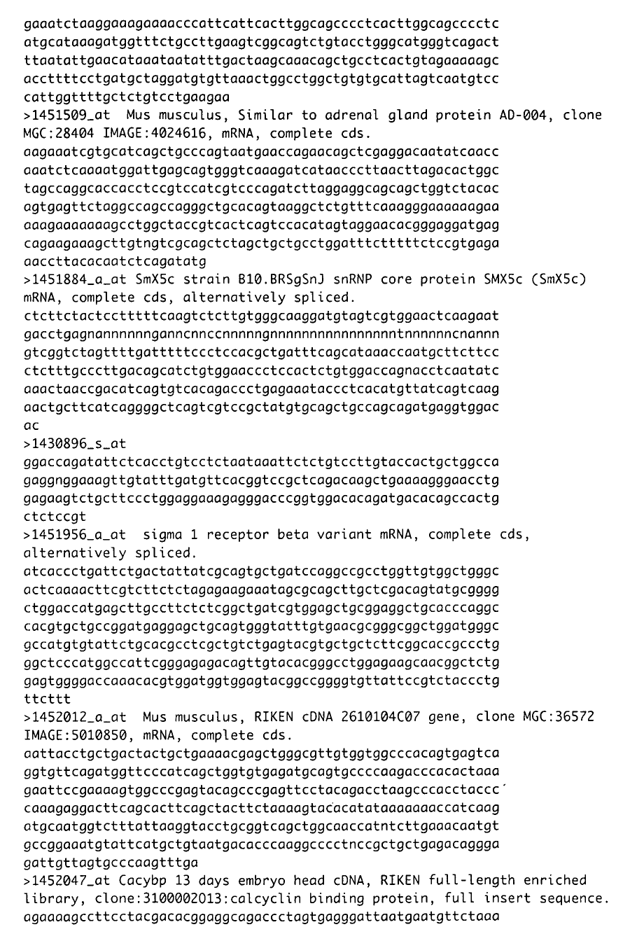

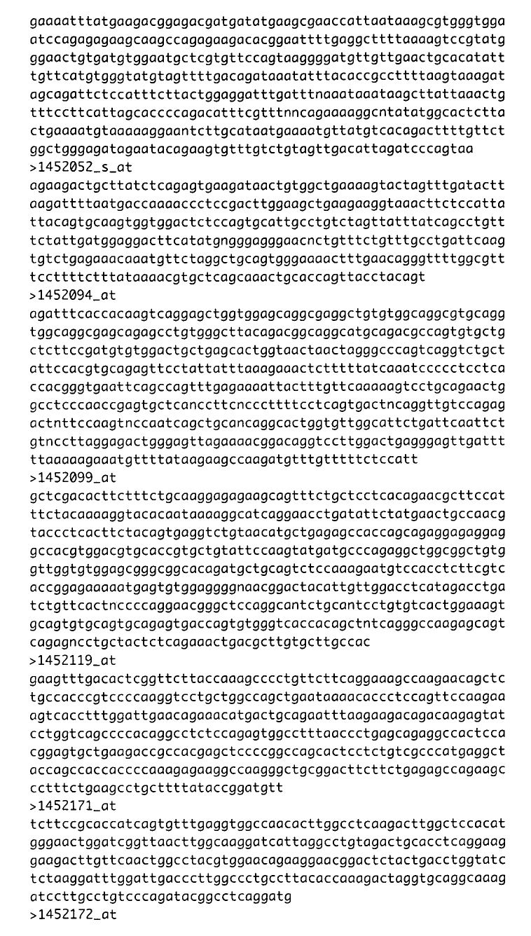

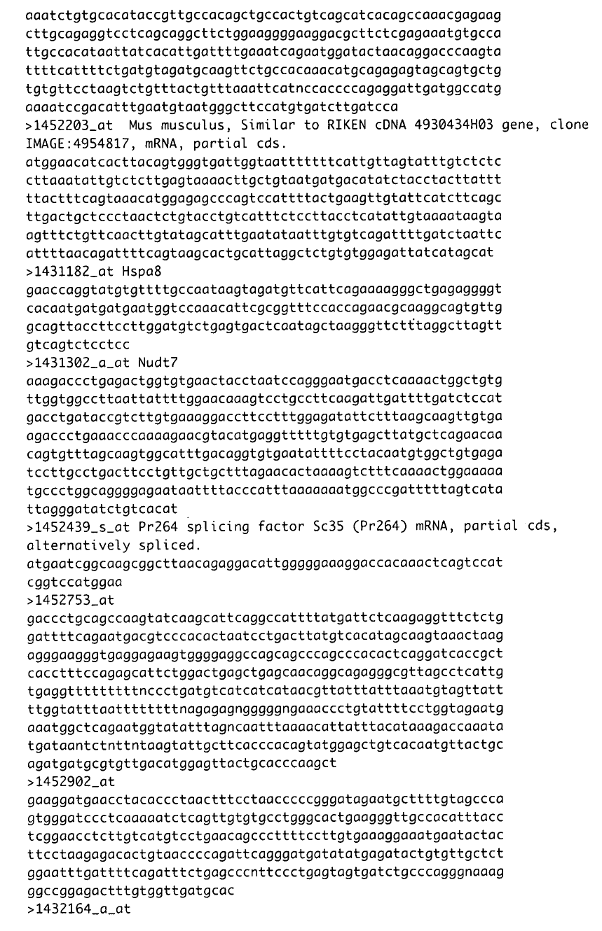

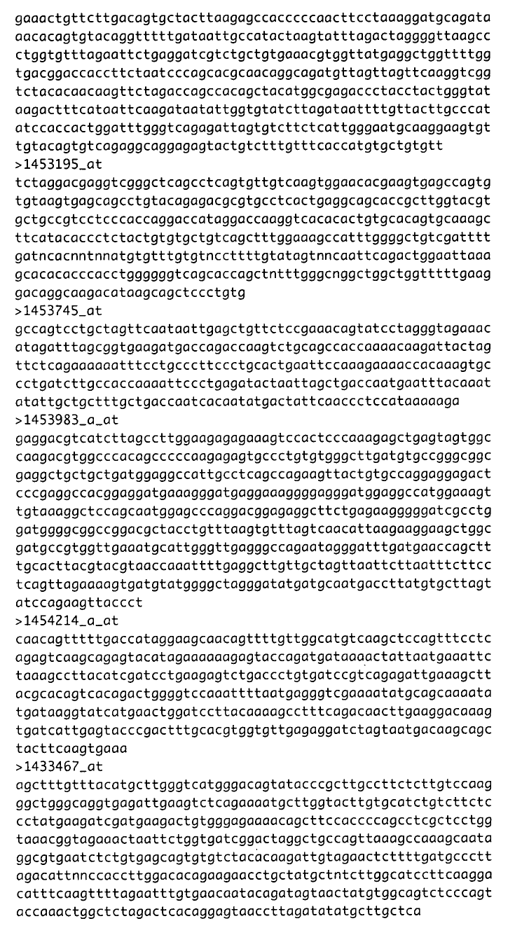

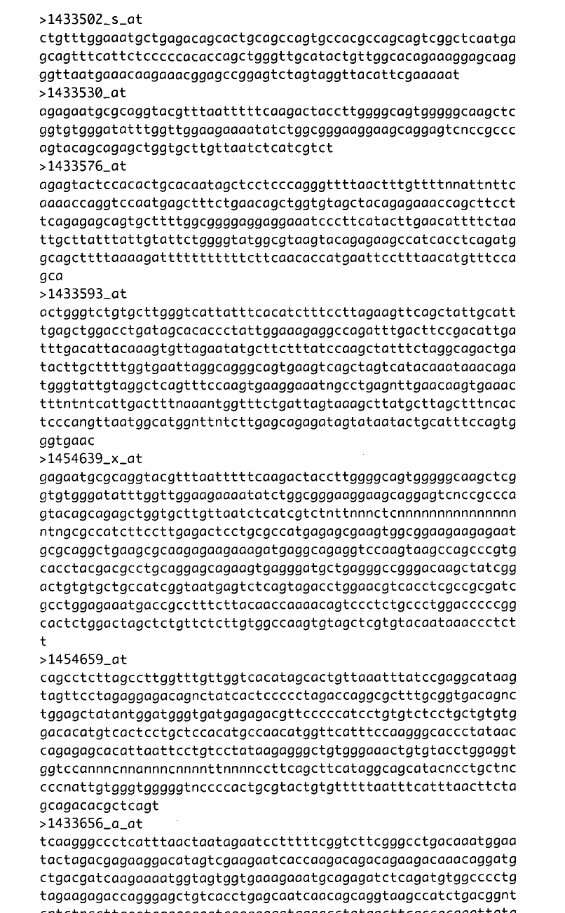

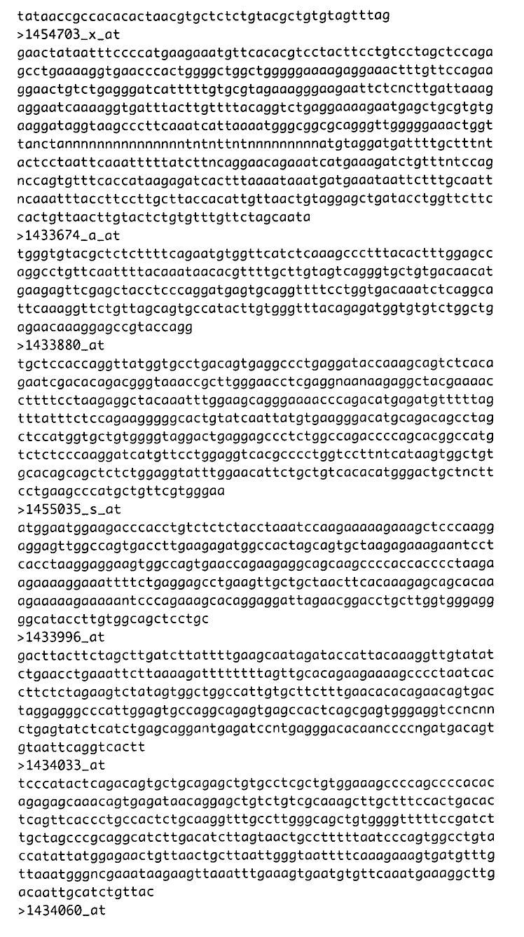

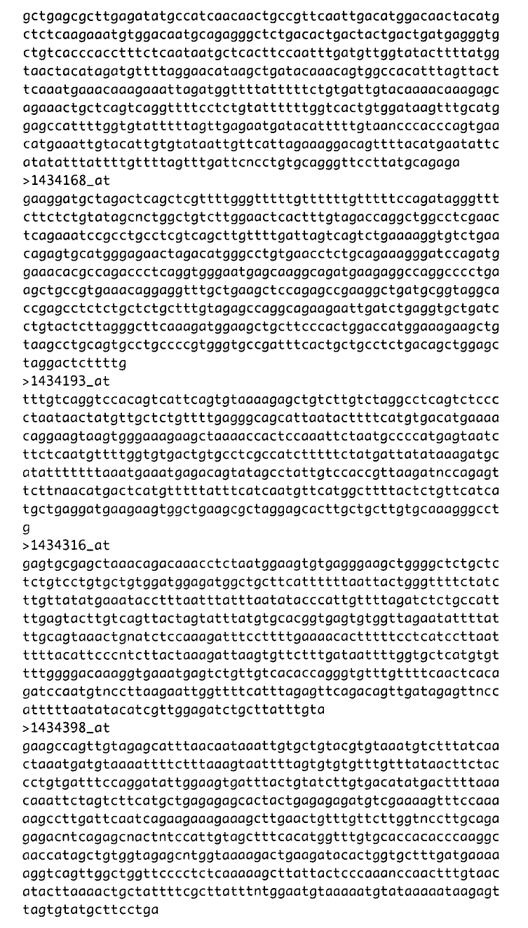

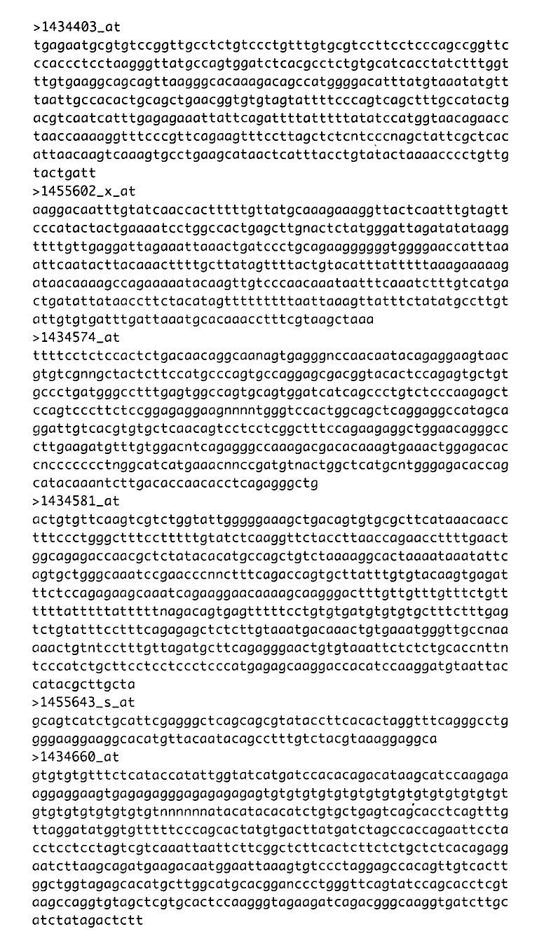

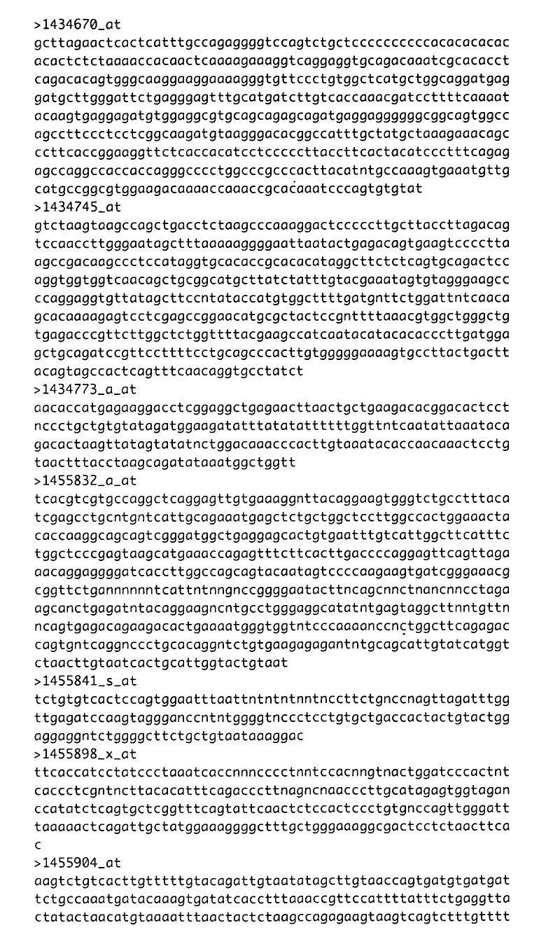

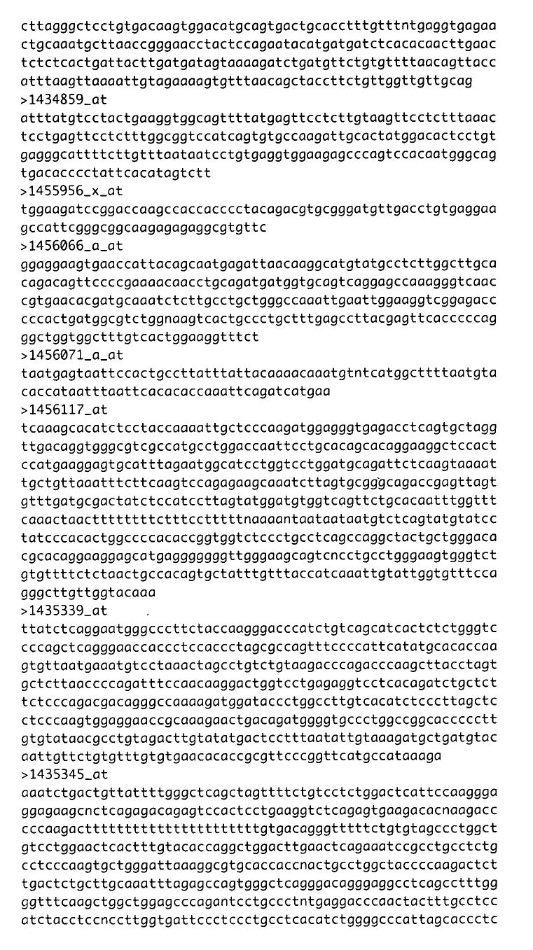

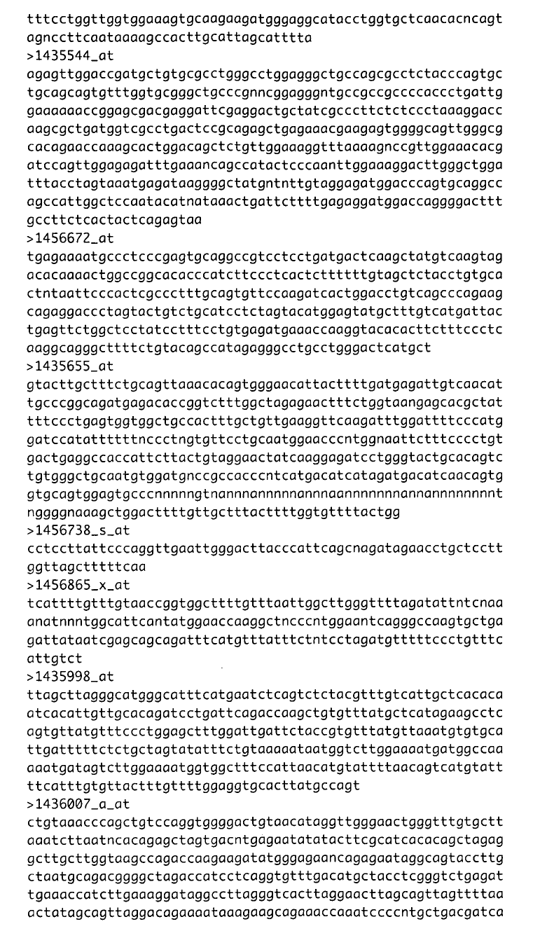

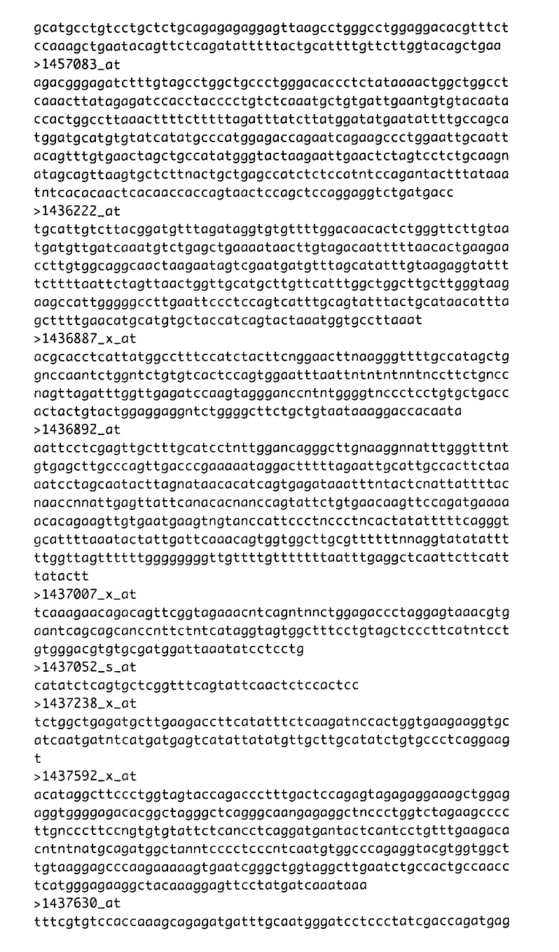

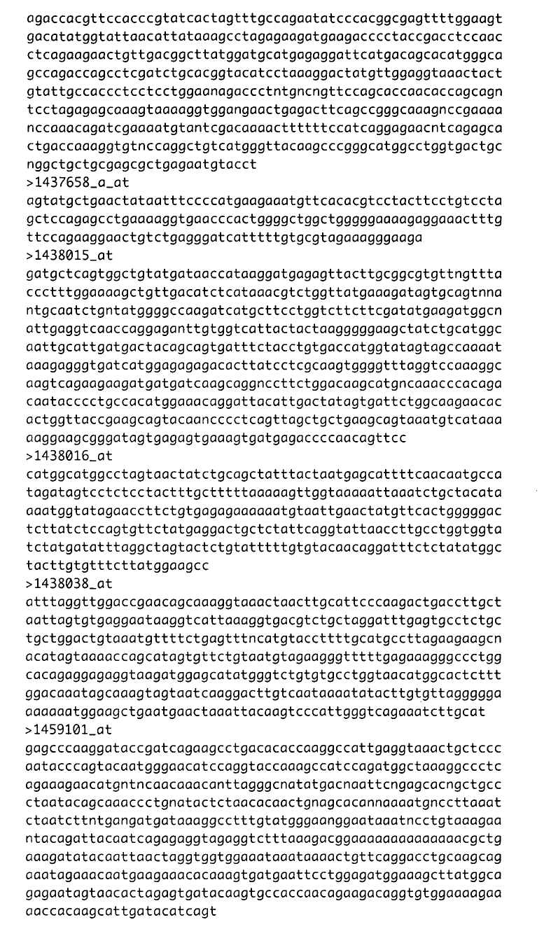

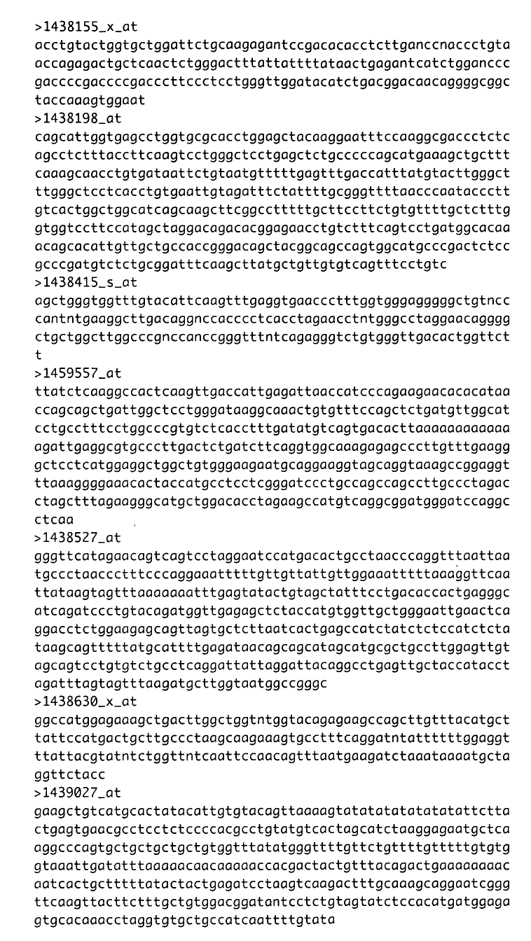

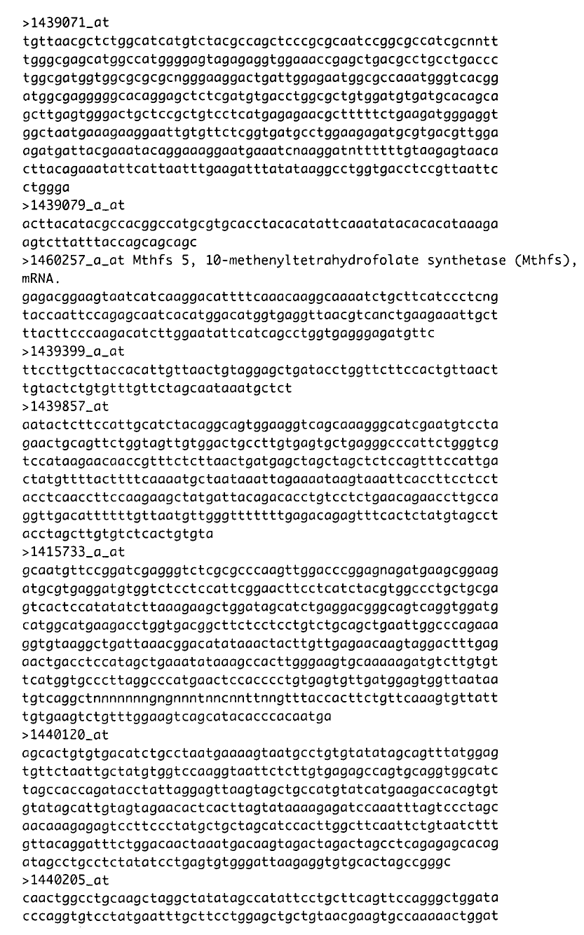

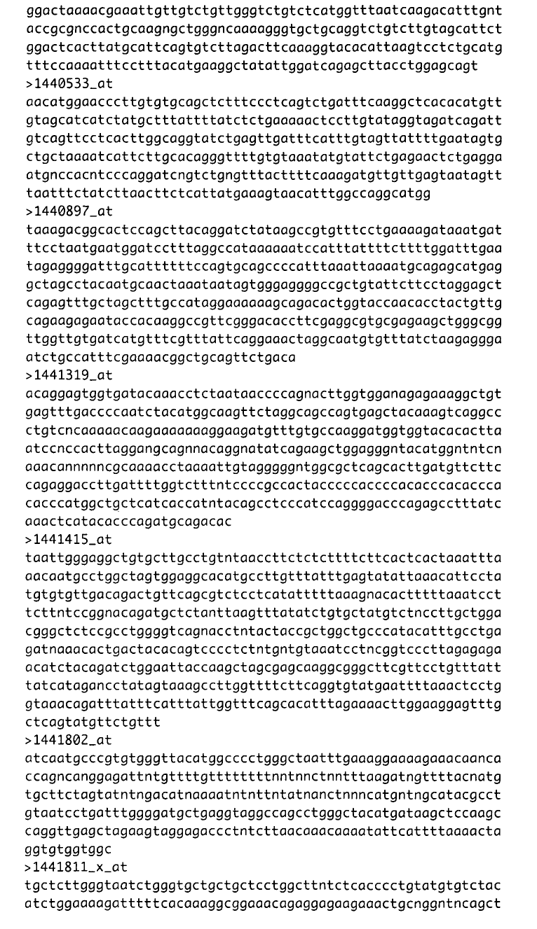

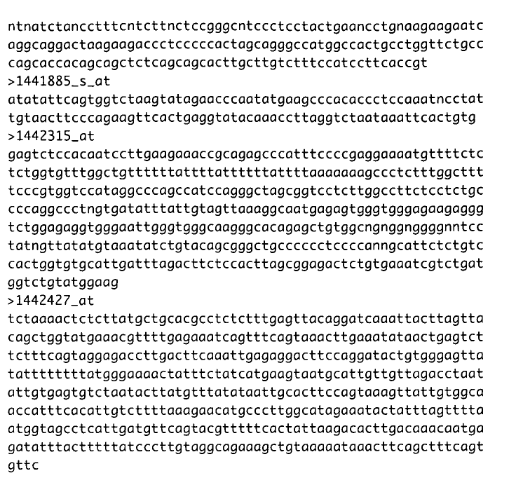

Gene expression profiles of Ba/F3 cells transfected by fusion or mutated kinases (9 samples: BCR-ABL, 2 BCR-FGFR1, 2 CEP1-FGFR1, 2 FOP-FGFR1, 2 V617F JAK2) were compared to that of control cells (6 samples) including parental Ba/F3 cells (4 samples) and Ba/F3 expressing a kinase-defective mutant of FOP-FGFR1 (2 samples). Supervised analysis, based on 17,885 filtered probe-sets, identified 294 differentially expressed probe sets (theoretical number of produced false positives=1.7) (FIGS. 1A, B), representing 228 genes and 8 ESTs, of which 188 were upregulated and 48 downregulated in activated kinase-expressing cells (Tables 2 and 3).

To translate the RNA expression profiles into functionality, discriminator genes/ESTs were interrogated by Onto-Express.⁷ Table 1 represents the most significant (p-value inferior at 3·10⁻²) and most often represented (including at least 3 genes) biological processes. Many of the upregulated genes encode nucleolar proteins involved in “ribosome biogenesis” (GO:0007046, 6 genes, p=4.28·10⁻¹¹), “rRNA processing” (GO:0006364; 7 genes, p=3.07·10⁻¹¹), and “protein biosynthesis” (GO:0006412, 9 genes, p=3.60·10⁻⁰⁵). Upregulated genes encode nucleolar proteins (CIRH1A, LARP1, NOL1, NOL11, NOL5, NOL5A, NOLA1, NOLA2, NOLC1, MKI67IP, SFRS2, SURF6), ribosomal proteins (RPL3, RPL12, RPL41, RPS9, RRS1), small nuclear ribonucleoproteins and interactors (U3/MPHOSPH10, LSM2, RNU22, RNU3IP2), components of RNA polymerase I (POLR1A, POLR1B), II (POLR2H, TAF9) and III (POLR3E, POLR3H), DEAD-box (DDX18, DDX56) and WD repeat (WDR4, WDR43, WDR74, WDR77, GRWD1, PWP1) proteins, eukaryotic initiation and elongation factors (EIF1A, EIF3S1, EIF3S4, EEF1E1), and components of the exosome (EXOSC1, EXOSC2, EXOSC6). Upregulated genes also encode proteins of the NOL5A-associated preribosomal ribonucleoprotein complex involved in pre-rRNA processing: NOL5A, PPAN, NOLC1, and BXDC2. The gene encoding EBNA1BP2 was upregulated; it encodes a protein that binds to nucleolar FGF3 and is regularly upregulated in tumors. The most upregulated sequence was GAS5, a non-protein-coding multiple small nucleolar RNA (snoRNA).

Other significant processes included “protein folding” (GO:0006457; 4 genes, p=6.72·10⁻⁰³), “ubiquitin-dependent protein catabolism” (GO:0006511, 3 genes, p=1.47·10⁻⁰²), “nuclear mRNA splicing, via spliceosome” (GO:0000398, 3 genes, p=1.65·10⁻⁰²), and “regulation of cell cycle” (GO:0000074, 3 genes, p=1.65·10⁻⁰²). The second major category of upregulated genes encode CCND2 (cyclin D2) and CDC25A, two major regulators needed for G1 progression. CCND2 RNA was found upregulated by BCR-ABL in previous gene expression studies. Cyclin D2 is necessary for BCR-ABL-induced activity. Inhibition of V617F JAK2 correlates with decreased expression of cyclin D2. Other G1 cyclins may play a role in the oncogenic activity of fusion kinases but cyclin D2 seems to be a rate-limiting element. We used Western blot analysis to validate the differential expression of cyclin D2. The amount of cyclin D2 protein was increased in Ba/F3 cells expressing activated kinases as compared to controls (FIG. 1C), in agreement with mRNA expression results.

MYC directly or indirectly regulates the G1 phase of the cell cycle. The list of upregulated genes included MYC. Many genes upregulated by MYC and NMYC oncogenes were also upregulated in our experiments, including CCND2, CDC25A and others (DDX18, EBNA1BP2, EEF1E1, MAT2A, MKI67IP, NOL5A, NOLA1, PHB, SFRS2, SHMT1, SLC16A1, SURF6, SRM, RPL3, RPL12, RPL41, RPS9 and RRS1). This similarity suggests that MYC proteins and MPD kinases have similar oncogenic effects, whose main target would be the CDKN2-RB protein pathway during the G1 phase of the cell cycle. Once induced, MYC may in turn act on the transcription of G1/S regulators and genes involved in protein synthesis. MPD fusion kinases are thought to target the hematopoietic stem cell. Activation of MYC is in perfect agreement with what we know of stem cell proliferation. A similar program was also turned on by IL3 stimulation (not shown).

Downregulated genes were more difficult to classify with Onto-Express, but several encode proteins with known or potential inhibitory function such as PIAS3, an inhibitor of STAT3, one of the main substrates of MPD kinases, and regulator of CDC25A, Erbin, and PLZF/ZBTB16, a MYC repressor.

We tested the validity of our classification by the “leave-one-out” cross-validation method. Iteratively, one of the 15 samples was removed, and a multigene predictor was generated from the remaining samples: 93% of samples were correctly assigned by the predictors with a sensitivity of 89% and a specificity of 100%.

Thus, in Ba/F3 cells, MPD fusion kinases induce both G1 activators and protein synthesis components, thus starting the cell proliferation machinery. This effect may be mediated by the PI3 kinase-AKT-TOR pathway, which controls and coordinates both protein synthesis and early phases of the cell cycle. Prominent downstream targets of the AKT pathway are cyclins D1, D2 and MYC.

The subject matter of the references set forth below are hereby incorporated by reference in their entirety:
1. Percy M J, McMullin M F. The V617F JAK2 mutation and the myeloproliferative disorders. Hematol Oncol 2005; 23: 91-93.
2. Delaval B, Letard S, Lelievre H, Chevrier V, Daviet L, Dubreuil P, et al. Oncogenic tyrosine kinase of malignant hemopathy targets the centrosome. Cancer Res 2005; 65: 7231-7240.
3. De Keersmaecker K, Cools J. Chronic myeloproliferative disorders: a tyrosine kinase tale. Leukemia 2006; 20: 200-205.
4. Guasch G, Ollendorff V, Borg J P, Birnbaum D, Pebusque M J. 8p12 stem cell myeloproliferative disorder: the FOP-fibroblast growth factor receptor 1 fusion protein of the t(6;8) translocation induces cell survival mediated by mitogen-activated protein kinase and phosphatidylinositol 3-kinase/Akt/mTOR pathways. Mol Cell Biol 2001; 21: 8129-8142.
5. Staerk J, Kallin A, Demoulin J B, Vainchenker W, Constantinescu S N. JAK1 and Tyk2 activation by the homologous polycythemia vera JAK2 V617F mutation: cross-talk with IGF1 receptor. J Biol Chem 2005; 280: 41893-41899.
6. James C, Ugo V, Le Couedic J P, Staerk J, Delhommeau F, Lacout C, et al. A unique clonal JAK2 mutation leading to constitutive signalling causes polycythaemia vera. Nature 2005; 434: 1144-1148.
7. Lamond A, Mann M. Nucleolar Proteome Database v 2.0. 2005 [cited september 06]; Available from: www.lamondlab.com/NoPDB.
8. Butler J S. The yin and yang of the exosome. Trends Cell Biol 2002; 12: 90-96.
9. Hayano T, Yanagida M, Yamauchi Y, Shinkawa T, Isobe T, Takahashi N. Proteomic analysis of human Nop56p-associated pre-ribosomal ribonucleoprotein complexes. Possible link between Nop56p and the nucleolar protein treacle responsible for Treacher Collins syndrome. J Biol Chem 2003; 278: 34309-34319.
10. Reimers K, Antoine M, Zapatka M, Blecken V, Dickson C, Kiefer P. NoBP, a nuclear fibroblast growth factor 3 binding protein, is cell cycle regulated and promotes cell growth. Mol Cell Biol 2001; 21: 4996-5007.
11. Pilarsky C, Wenzig M, Specht T, Saeger H D, Grutzmann R. Identification and validation of commonly overexpressed genes in solid tumors by comparison of microarray data. Neoplasia 2004; 6: 744-750.
12. Smith C M, Steitz J A. Classification of gas5 as a multi-small-nucleolar-RNA (snoRNA) host gene and a member of the 5′-terminal oligopyrimidine gene family reveals common features of snoRNA host genes. Mol Cell Biol 1998; 18: 6897-6909.
13. Parada Y, Banerji L, Glassford J, Lea N C, Collado M, Rivas C, et al. BCR-ABL and interleukin 3 promote haematopoietic cell proliferation and survival through modulation of cyclin D2 and p27Kip1 expression. J Biol Chem 2001; 276: 23572-23580.
14. Deininger M W, Vieira S, Mendiola R, Schultheis B, Goldman J M, Melo J V. BCR-ABL tyrosine kinase activity regulates the expression of multiple genes implicated in the pathogenesis of chronic myeloid leukemia. Cancer Res 2000; 60: 2049-2055.
15. Jena N, Deng M, Sicinska E, Sicinski P, Daley G Q. Critical role for cyclin D2 in BCR/ABL-induced proliferation of hematopoietic cells. Cancer Res 2002; 62: 535-541.
16. Walz C, Crowley B J, Hudon H E, Gramlich J L, Neuberg D S, Podar K, et al. Activated JAK2 with the V617F point mutation promotes G1/S-phase transition. J Biol Chem 2006.
17. Nasi S, Ciarapica R, Jucker R, Rosati J, Soucek L. Making decisions through Myc. FEBS Lett 2001; 490: 153-162.
18. Bouchard C, Thieke K, Maier A, Saffrich R, Hanley-Hyde J, Ansorge W, et al. Direct induction of cyclin D2 by Myc contributes to cell cycle progression and sequestration of p27. Embo J 1999; 18: 5321-5333.
19. Perez-Roger I, Kim S H, Griffiths B, Sewing A, Land H. Cyclins D1 and D2 mediate myc-induced proliferation via sequestration of p27(Kip1) and p21(Cip1). Embo J 1999; 18: 5310-5320.
20. Coller H A, Grandori C, Tamayo P, Colbert T, Lander E S, Eisenman R N, et al. Expression analysis with oligonucleotide microarrays reveals that MYC regulates genes involved in growth, cell cycle, signaling, and adhesion. Proc Natl Acad Sci USA 2000; 97: 3260-3265.
21. Zeller K I, Jegga A G, Aronow B J, O'Donnell K A, Dang C V. An integrated database of genes responsive to the Myc oncogenic transcription factor: identification of direct genomic targets. Genome Biol 2003; 4: R69.
22. Schlosser I, Holzel M, Hoffmann R, Burtscher H, Kohlhuber F, Schuhmacher M, et al. Dissection of transcriptional programmes in response to serum and c-Myc in a human B-cell line. Oncogene 2005; 24: 520-524.
23. Boon K, Caron H N, van Asperen R, Valentijn L, Hermus M C, van Sluis P, et al. N-myc enhances the expression of a large set of genes functioning in ribosome biogenesis and protein synthesis. Embo J 2001; 20: 1383-1393.
24. Steiner P, Rudolph B, Muller D, Eilers M. The functions of Myc in cell cycle progression and apoptosis. Prog Cell Cycle Res 1996; 2: 73-82.
25. Moore K A, Lemischka I R. Stem cells and their niches. Science 2006; 311: 1880-1885.
26. Barre B, Vigneron A, Coqueret O. The STAT3 transcription factor is a target for the Myc and riboblastoma proteins on the Cdc25A promoter. J Biol Chem 2005; 280: 15673-15681.
27. McConnell M J, Chevallier N, Berkofsky-Fessler W, Giltnane J M, Malani R B, Staudt L M, et al. Growth suppression by acute promyelocytic leukemia-associated protein PLZF is mediated by repression of c-myc expression. Mol Cell Biol 2003; 23: 9375-9388.
28. Golub T R, Slonim D K, Tamayo P, Huard C, Gaasenbeek M, Mesirov J P, et al. Molecular classification of cancer: class discovery and class prediction by gene expression monitoring. Science 1999; 286: 531-537.
29. Ruggero D, Sonenberg N. The Akt of translational control. Oncogene 2005; 24: 7426-7434.
30. Gera J F, Mellinghoff I K, Shi Y, Rettig M B, Tran C, Hsu J H, et al. AKT activity determines sensitivity to mammalian target of rapamycin (mTOR) inhibitors by regulating cyclin D1 and c-myc expression. J Biol Chem 2004; 279: 2737-2746.
31. Malumbres M, Barbacid M. To cycle or not to cycle: a critical decision in cancer. Nat Rev Cancer 2001; 1: 222-231.

TABLE 1
Selection of discriminant genes classified by Onto-Express

| | | | | | Discriminating |
|---|---|---|---|---|---|
| GO ID | Biological process | P-value | Gene Symbol | Probe set | Score |
| GO:0006364 | rRNA processing | 3.07E−11 | Nola1 | 1418305_s_at | 2.10 |
| | | | Exosc2 | 1426630_at | 1.96 |
| | | | Rnu3ip2 | 1451293_at | 1.46 |
| | | | Mphosph10 | 1429080_at | 1.42 |
| | | | Ddx56 | 1423815_at | 1.41 |
| | | | Ebna1bp2 | 1428315_at | 1.31 |
| | | | Exosc1 | 1452012_a_at | 1.31 |
| GO:0007046 | ribosome biogenesis | 4.82E−11 | Rpl12 | 1435655_at | 1.73 |
| | | | 1110017C15Rik | 1448480_at | 1.63 |
| | | | Rrs1 | 1456865_x_at | 1.63 |
| | | | Nol5a | 1426533_at | 1.59 |
| | | | Gtpbp4 | 1450873_at | 1.40 |
| | | | 2610012O22Rik | 1423823_at | 1.30 |
| GO:0006412 | protein biosynthesis | 3.60E−05 | Rpl41 | 1454639_x_at | 1.90 |
| | | | Mrps18b | 1451164_a_at | 1.82 |
| | | | Rpl3 | 1438527_at | 1.79 |
| | | | Rps9 | 1426958_at | 1.69 |
| | | | Eif3s1 | 1426394_at | 1.53 |
| | | | Nola2 | 1416605_at | 1.51 |
| | | | Itgb4bp | 1427578_a_at | 1.47 |
| | | | Eef1e1 | 1449044_at | 1.45 |
| | | | Eif3s4 | 1417718_at | 1.38 |
| GO:0006457 | protein folding | 6.72E−03 | Dnajc11 | 1433880_at | 1.38 |
| | | | Cct3 | 1448178_a_at | 1.33 |
| | | | Ero1l | 1419030_at | 1.32 |
| | | | Hyou1 | 1423291_s_at | 1.31 |
| GO:0006511 | ubiquitin-dependent | 1.47E−02 | Usp39 | 1437007_x_at | 1.51 |
| | protein catabolism | | Siah2 | 1448171_at | 1.30 |
| | | | Usp10 | 1448230_at | 1.29 |
| GO:0000398 | nuclear mRNA splicing, | 1.65E−02 | Mki67ip | 1424001_at | 1.88 |
| | via spliceosome | | Pprc1 | 1426381_at | 1.86 |
| | | | Sfrs2 | 1415807_s_at | 1.46 |
| GO:0000074 | regulation of cell cycle | 1.65E−02 | Axl | 1423586_at | 1.65 |
| | | | Ccnd2 | 1430127_a_at | 1.51 |
| | | | Cdc25a | 1417132_at | 1.43 |

TABLE 2
188 upregulated genes/EST associated with their biological process ordered by discriminating score

| | | | | | Onto- |
|---|---|---|---|---|---|
| | | Discriminating | | | Express P [symbol] |
| Probe set | Gene Symbol | Score | GO ID | Onto-Express Biological Process | value |
| 1455904_at | Gas5 | 3.32 | null | unknownP | 0.33 |
| 1435998_at | na | 3.03 | null | unknownP | 0.33 |
| 1416345_at | Timm8a | 2.57 | GO:0006626 | protein-mitochondrial targeting | 0.00 |
| 1455643_s_at | AW550801 | 2.32 | null | unknownP | 0.33 |
| 1429061_at | 1810063B05Rik | 2.29 | null | unknownP | 0.33 |
| 1425177_at | Shmt1 | 2.28 | GO:0006563 | L-serine metabolism | 0.00 |
| 1423138_at | Wdr4 | 2.25 | GO:0008033 | tRNA processing | 0.02 |
| 1416376_at | 1810014L12Rik | 2.21 | null | unknownP | 0.33 |
| 1456117_at | 2600005C20Rik | 2.14 | null | unknownP | 0.33 |
| 1428529_at | 2810026P18Rik | 2.12 | null | unknownP | 0.33 |
| 1418305_s_at | Nola1 | 2.10 | GO:0006364 | rRNA processing | 0.00 |
| 1451509_at | Taf9 | 2.10 | GO:0006352 | transcription initiation | 0.00 |
| 1454214_a_at | 2410019A14Rik | 2.10 | null | unknownP | 0.33 |
| 1425820_x_at | Gpatc4 | 2.08 | null | unknownP | 0.33 |
| 1433467_at | Slc7a6 | 2.03 | GO:0006810 | transport | 0.20 |
| 1441415_at | Spred2 | 2.02 | GO:0000188 | inactivation of MAPK | 0.00 |
| 1426630_at | Exosc2 | 1.96 | GO:0006364 | rRNA processing | 0.00 |
| 1437592_x_at | 1700023O11Rik | 1.93 | null | unknownP | 0.33 |
| 1424620_at | D13Wsu177e | 1.92 | null | unknownP | 0.33 |
| 1455832_a_at | Umps | 1.91 | GO:0006221 | pyrimidine nucleotide biosynthesis | 0.00 |
| 1421260_a_at | Srm | 1.91 | GO:0008295 | spermidine biosynthesis | 0.00 |
| 1428869_at | Nolc1 | 1.90 | GO:0007000 | nucleolus organization and biogenesis | 0.00 |
| 1454639_x_at | Rpl41 | 1.90 | GO:0006412 | protein biosynthesis | 0.00 |
| 1424001_at | Mki67ip | 1.88 | GO:0000398 | nuclear mRNA splicing, via spliceosome | 0.02 |
| 1435544_at | Exosc6 | 1.87 | GO:0000004 | biological process unknown | 0.02 |
| 1425830_a_at | 2810452K22Rik | 1.87 | null | unknownP | 0.33 |
| 1426381_at | Pprc1 | 1.86 | GO:0000398 | nuclear mRNA splicing, via spliceosome | 0.02 |
| 1438015_at | BC068171 | 1.84 | null | unknownP | 0.33 |
| 1451164_a_at | Mrps18b | 1.82 | GO:0006412 | protein biosynthesis | 0.00 |
| 1416890_at | Wdr74 | 1.81 | null | unknownP | 0.33 |
| 1415834_at | Dusp6 | 1.81 | GO:0006470 | protein amino acid dephosphorylation | 0.02 |
| 1438527_at | Rpl3 | 1.79 | GO:0006412 | protein biosynthesis | 0.00 |
| 1424151_at | MGI: 2385237 | 1.79 | GO:0000004 | biological process unknown | 0.02 |
| 1426554_a_at | Pgam1 | 1.79 | GO:0006096 | glycolysis | 0.04 |
| 1455841_s_at | Grwd1 | 1.77 | null | unknownP | 0.33 |
| 1417873_at | Pwp1 | 1.75 | null | unknownP | 0.33 |
| 1452099_at | AA408296 | 1.75 | null | unknownP | 0.33 |
| 1422484_at | Cycs | 1.74 | GO:0008635 | caspase activation via cytochrome c | 0.00 |
| 1416070_a_at | Ddx18 | 1.74 | null | unknownP | 0.33 |
| 1435655_at | Rpl12 | 1.73 | GO:0007046 | ribosome biogenesis | 0.00 |
| 1428390_at | Wdr43 | 1.72 | null | unknownP | 0.33 |
| 1434773_a_at | Slc2a1 | 1.71 | GO:0008643 | carbohydrate transport | 0.00 |
| 1429268_at | 2610318N02Rik | 1.70 | null | unknownP | 0.33 |
| 1426958_at | Rps9 | 1.69 | GO:0006412 | protein biosynthesis | 0.00 |
| 1437658_a_at | Rnu22 | 1.69 | null | unknownP | 0.33 |
| 1419518_at | Tuba8 | 1.69 | GO:0051258 | protein polymerization | 0.01 |
| 1423703_at | Ppan | 1.68 | GO:0001560 | regulation of cell growth by extracellular stimulus | 0.00 |
| 1426931_s_at | D19Bwg1357e | 1.67 | null | unknownP | 0.33 |
| 1418225_at | Orc2l | 1.67 | GO:0006260 | DNA replication | 0.03 |
| 1415802_at | Slc16a1 | 1.66 | GO:0015711 | organic anion transport | 0.00 |
| 1423586_at | Axl | 1.65 | GO:0000074 | regulation of cell cycle | 0.02 |
| 1450387_s_at | Ak3 | 1.65 | GO:0006139 | nucleobase, nucleoside, nucleotide and nucleic acid metabolism | 0.00 |
| 1423884_at | Cirh1a | 1.65 | null | unknownP | 0.33 |
| 1429897_a_at | D16Ertd472e | 1.64 | null | unknownP | 0.33 |
| 1416445_at | 2810405J04Rik | 1.63 | null | unknownP | 0.33 |
| 1448480_at | 1110017C15Rik | 1.63 | GO:0007046 | ribosome biogenesis | 0.00 |
| 1428970_at | Mak3 | 1.63 | null | unknownP | 0.33 |
| 1449886_a_at | Timm9 | 1.63 | GO:0006626 | protein-mitochondrial targeting | 0.00 |
| 1456865_x_at | Rrs1 | 1.63 | GO:0007046 | ribosome biogenesis | 0.00 |
| 1452902_at | 2610209N15Rik | 1.62 | GO:0008152 | metabolism | 0.05 |
| 1423161_s_at | Spred1 | 1.60 | GO:0000188 | inactivation of MAPK | 0.00 |
| 1448140_at | Ciapin1 | 1.60 | GO:0030097 | hemopoiesis | 0.02 |
| 1426533_at | Nol5a | 1.59 | GO:0007046 | ribosome biogenesis | 0.00 |
| 1427997_at | 1110007M04Rik | 1.58 | null | unknownP | 0.33 |
| 1448450_at | Ak2 | 1.58 | GO:0006139 | nucleobase, nucleoside, nucleotide and nucleic acid metabolism | 0.00 |
| 1446771_at | na | 1.57 | null | unknownP | 0.33 |
| 1452047_at | Cacybp | 1.55 | GO:0006512 | ubiquitin cycle | 0.12 |
| 1417064_at | Jagn1 | 1.55 | null | unknownP | 0.33 |
| 1424344_s_at | Eif1a | 1.54 | null | unknownP | 0.33 |
| 1424473_at | Polr2h | 1.54 | GO:0006350 | transcription | 0.14 |
| 1433996_at | Suv39h2 | 1.54 | GO:0006333 | chromatin assembly or disassembly | 0.02 |
| 1426394_at | Eif3s1 | 1.53 | GO:0006412 | protein biosynthesis | 0.00 |
| 1428694_at | 5033413D16Rik | 1.53 | null | unknownP | 0.33 |
| 1434033_at | Tle1 | 1.53 | GO:0007222 | frizzled signaling pathway | 0.01 |
| 1425921_a_at | 1810055G02Rik | 1.52 | null | unknownP | 0.33 |
| 1440120_at | Gnb2l1 | 1.52 | GO:0007205 | protein kinase C activation | 0.01 |
| 1452094_at | P4ha1 | 1.52 | GO:0018401 | peptidyl-proline hydroxylation to 4-hydroxy-L-proline | 0.00 |
| 1439071_at | 5430416N02Rik | 1.51 | null | unknownP | 0.33 |
| 1437007_x_at | Usp39 | 1.51 | GO:0006511 | ubiquitin-dependent protein catabolism | 0.01 |
| 1416605_at | Nola2 | 1.51 | GO:0006412 | protein biosynthesis | 0.00 |
| 1433576_at | Mat2a | 1.51 | GO:0006556 | S-adenosylmethionine biosynthesis | 0.00 |
| 1430127_a_at | Ccnd2 | 1.51 | GO:0000074 | regulation of cell cycle | 0.02 |
| 1417675_a_at | Mdn1 | 1.51 | null | unknownP | 0.33 |
| 1429612_at | Eml4 | 1.50 | GO:0000004 | biological process unknown | 0.02 |
| 1450011_at | Hsd17b12 | 1.50 | GO:0006694 | steroid biosynthesis | 0.04 |
| 1451254_at | Ikbkap | 1.50 | GO:0000004 | biological process unknown | 0.02 |
| 1448563_at | Phb | 1.50 | GO:0006259 | DNA metabolism | 0.04 |
| 1432164_a_at | Gcsh | 1.49 | GO:0006546 | glycine catabolism | 0.00 |
| 1423730_at | C130052I12Rik | 1.49 | null | unknownP | 0.33 |
| 1420056_s_at | Ptdsr | 1.49 | GO:0006915 | apoptosis | 0.04 |
| 1450914_at | Ppp1r14b | 1.48 | null | unknownP | 0.33 |
| 1427578_a_at | Itgb4bp | 1.47 | GO:0006412 | protein biosynthesis | 0.00 |
| 1453745_at | 2700038G22Rik | 1.47 | null | unknownP | 0.33 |
| 1415807_s_at | Sfrs2 | 1.46 | GO:0000398 | nuclear mRNA splicing, via spliceosome | 0.02 |
| 1447403_a_at | Zmynd19 | 1.46 | null | unknownP | 0.33 |
| 1451293_at | Rnu3ip2 | 1.46 | GO:0006364 | rRNA processing | 0.00 |
| 1434574_at | 9430008C03Rik | 1.46 | null | unknownP | 0.33 |
| 1424545_at | BC003965 | 1.45 | null | unknownP | 0.33 |
| 1449044_at | Eef1e1 | 1.45 | GO:0006412 | protein biosynthesis | 0.00 |
| 1435339_at | Kctd15 | 1.45 | null | unknownP | 0.33 |
| 1453195_at | Sdccag3 | 1.44 | null | unknownP | 0.33 |
| 1417132_at | Cdc25a | 1.43 | GO:0000087 | M phase of mitotic cell cycle | 0.00 |
| 1434398_at | 9430034D17Rik | 1.43 | GO:0006355 | regulation of transcription, DNA-dependent | 0.05 |
| 1453983_a_at | 2810013M15Rik | 1.43 | null | unknownP | 0.33 |
| 1417233_at | Chchd4 | 1.43 | GO:0000004 | biological process unknown | 0.02 |
| 1448135_at | Atf4 | 1.43 | GO:0006094 | gluconeogenesis | 0.01 |
| 1424942_a_at | Myc | 1.42 | GO:0008633 | activation of pro-apoptotic gene products | 0.00 |
| 1424019_at | Nol1 | 1.42 | GO:0000004 | biological process unknown | 0.02 |
| 1416962_at | Rcc1 | 1.42 | null | unknownP | 0.33 |
| 1429080_at | Mphosph10 | 1.42 | GO:0006364 | rRNA processing | 0.00 |
| 1422844_a_at | Wdr77 | 1.41 | null | unknownP | 0.33 |
| 1419058_at | Praf1 | 1.41 | null | unknownP | 0.33 |
| 1423815_at | Ddx56 | 1.41 | GO:0006364 | rRNA processing | 0.00 |
| 1416864_at | Surf6 | 1.41 | null | unknownP | 0.33 |
| 1416559_at | 1500003O22Rik | 1.40 | null | unknownP | 0.33 |
| 1434316_at | Chsy1 | 1.40 | GO:0030206 | chondroitin sulfate biosynthesis | 0.00 |
| 1449348_at | Mpp6 | 1.40 | null | unknownP | 0.33 |
| 1450873_at | Gtpbp4 | 1.40 | GO:0007046 | ribosome biogenesis | 0.00 |
| 1451459_at | Ahctf1 | 1.40 | null | unknownP | 0.33 |
| 1433530_at | 2210411K19Rik | 1.40 | null | unknownP | 0.33 |
| 1418571_at | Tnfrsf12a | 1.39 | GO:0006931 | substrate-bound cell migration, cell attachment to substrate | 0.00 |
| 1437238_x_at | Nmd3 | 1.39 | null | unknownP | 0.33 |
| 1428248_at | Nfx1 | 1.39 | GO:0045347 | negative regulation of MHC class II biosynthesis | 0.00 |
| 1448617_at | Cd53 | 1.39 | null | unknownP | 0.33 |
| 1424227_at | Polr3h | 1.39 | GO:0006101 | citrate metabolism | 0.00 |
| 1433656_a_at | Gnl3 | 1.39 | null | unknownP | 0.33 |
| 1426939_at | 2310007F12Rik | 1.39 | null | unknownP | 0.33 |
| 1418079_at | Psme3 | 1.39 | null | unknownP | 0.33 |
| 1448126_at | MGI: 1929091 | 1.38 | GO:0000004 | biological process unknown | 0.02 |
| 1415733_a_at | 1110019J04Rik | 1.38 | null | unknownP | 0.33 |
| 1433880_at | Dnajc11 | 1.38 | GO:0006457 | protein folding | 0.01 |
| 1417718_at | Eif3s4 | 1.38 | GO:0006412 | protein biosynthesis | 0.00 |
| 1416563_at | Ctps | 1.38 | GO:0006221 | pyrimidine nucleotide biosynthesis | 0.00 |
| 1426311_s_at | Zdhhc5 | 1.37 | null | unknownP | 0.33 |
| 1416750_at | Oprs1 | 1.37 | null | unknownP | 0.33 |
| 1448568_a_at | Slc20a1 | 1.36 | GO:0006817 | phosphate transport | 0.04 |
| 1452753_at | Foxk2 | 1.36 | null | unknownP | 0.33 |
| 1420463_at | MGI: 1351468 | 1.36 | GO:0007169 | transmembrane receptor protein tyrosine kinase signaling pathway | 0.07 |
| 1425837_a_at | Ccrn4l | 1.36 | GO:0048511 | rhythmic process | 0.01 |
| 1434168_at | Peo1 | 1.36 | GO:0006268 | DNA unwinding | 0.00 |
| 1416448_at | Itpa | 1.36 | GO:0009117 | nucleotide metabolism | 0.02 |
| 1434660_at | Alkbh | 1.36 | GO:0000004 | biological process unknown | 0.02 |
| 1417035_at | Sac3d1 | 1.35 | null | unknownP | 0.33 |
| 1423480_at | Nol11 | 1.35 | null | unknownP | 0.33 |
| 1424436_at | Gart | 1.35 | GO:0009113 | purine base biosynthesis | 0.00 |
| 1450698_at | Dusp2 | 1.35 | GO:0006470 | protein amino acid dephosphorylation | 0.02 |
| 1454659_at | Dctd | 1.35 | GO:0006220 | pyrimidine nucleotide metabolism | 0.00 |
| 1438198_at | Bri3bp | 1.35 | null | unknownP | 0.33 |
| 1431182_at | Hspa8 | 1.34 | GO:0051085 | chaperone cofactor dependent protein folding | 0.00 |
| 1426426_at | Rbm13 | 1.34 | null | unknownP | 0.33 |
| 1416126_at | Rpo1-2 | 1.34 | GO:0006350 | transcription | 0.14 |
| 1424522_at | Heatr1 | 1.34 | null | unknownP | 0.33 |
| 1428244_at | Larp1 | 1.33 | null | unknownP | 0.33 |
| 1429456_a_at | Polr3e | 1.33 | GO:0006350 | transcription | 0.14 |
| 1456066_a_at | Rpo1-4 | 1.33 | GO:0006350 | transcription | 0.14 |
| 1448178_a_at | Cct3 | 1.33 | GO:0006457 | protein folding | 0.01 |
| 1451385_at | 2310056P07Rik | 1.33 | null | unknownP | 0.33 |
| 1440205_at | na | 1.32 | null | unknownP | 0.33 |
| 1418566_s_at | Nudcd2 | 1.32 | null | unknownP | 0.33 |
| 1419030_at | Ero1l | 1.32 | GO:0006457 | protein folding | 0.01 |
| 1452203_at | 5830411E10Rik | 1.32 | GO:0006260 | DNA replication | 0.03 |
| 1437052_s_at | Slc2a3 | 1.32 | GO:0008643 | carbohydrate transport | 0.00 |
| 1417726_at | Sssca1 | 1.32 | null | unknownP | 0.33 |
| 1422767_at | Bysl | 1.31 | GO:0007155 | cell adhesion | 0.34 |
| 1423291_s_at | Hyou1 | 1.31 | GO:0006457 | protein folding | 0.01 |
| 1452172_at | 2810421I24Rik | 1.31 | null | unknownP | 0.33 |
| 1437630_at | D16Bwg1547e | 1.31 | null | unknownP | 0.33 |
| 1424244_at | Rwdd4a | 1.31 | null | unknownP | 0.33 |
| 1417212_at | 9530058B02Rik | 1.31 | null | unknownP | 0.33 |
| 1428315_at | Ebna1bp2 | 1.31 | GO:0006364 | rRNA processing | 0.00 |
| 1436007_a_at | Thumpd1 | 1.31 | GO:0000004 | biological process unknown | 0.02 |
| 1439027_at | C330023M02Rik | 1.31 | null | unknownP | 0.33 |
| 1452012_a_at | Exosc1 | 1.31 | GO:0006364 | rRNA processing | 0.00 |
| 1450986_at | Nol5 | 1.31 | GO:0006608 | snRNP protein-nucleus import | 0.00 |
| 1423705_at | 2310057D15Rik | 1.30 | GO:0008152 | metabolism | 0.05 |
| 1456738_s_at | Brp16 | 1.30 | GO:0000004 | biological process unknown | 0.02 |
| 1451884_a_at | Lsm2 | 1.30 | null | unknownP | 0.33 |
| 1421089_a_at | 2610028A01Rik | 1.30 | null | unknownP | 0.33 |
| 1451016_at | Ifrd2 | 1.30 | GO:0030154 | cell differentiation | 0.10 |
| 1423841_at | Bxdc2 | 1.30 | null | unknownP | 0.33 |
| 1416022_at | Fabp5 | 1.30 | GO:0006656 | phosphatidylcholine biosynthesis | 0.00 |
| 1423823_at | 2610012O22Rik | 1.30 | GO:0007046 | ribosome biogenesis | 0.00 |
| 1448171_at | Siah2 | 1.30 | GO:000651 1 | ubiquitin-dependent protein catabolism | 0.01 |
| 1448413_at | 2410016O06Rik | 1.29 | GO:0042254 | ribosome biogenesis and assembly | 0.00 |
| 1456672_at | AA408556 | 1.29 | null | unknownP | 0.33 |
| 1416442_at | Ier2 | 1.29 | GO:0000004 | biological process unknown | 0.02 |
| 1457083_at | Prpf31 | 1.29 | GO:0000351 | assembly of spliceosomal tri-snRNP U4/U6.U5 | 0.00 |
| 1448230_at | Usp10 | 1.29 | GO:0006511 | ubiquitin-dependent protein catabolism | 0.01 |

[symbol]

indicates data missing or illegible when filed

TABLE 3
48 genes/EST downregulated in fusion kinase-expressing Ba/F3 cells.
Probe sets are ordered by increasing discriminating s

[symbol]

| | | | | | Onto- |
|---|---|---|---|---|---|
| | | Discriminating | | | Express P [symbol] |
| Probe Set | Gene Symbol | Score | GO ID | Onto-Express Biological Process | value |
| 1438038_at | 4930402H24Rik | −2.301840428 | null | unknownP | 2.04E−01 |
| 1459101_at | C78760 | −1.937260526 | null | unknownP | 2.04E−01 |
| 1430185_at | 5830460E08Rik | −1.899962271 | null | unknownP | 2.04E−01 |
| 1459557_at | Zbtb16 | −1.889802945 | GO:0035136 | forelimb morphogenesis | 8.32E−05 |
| 1425603_at | 0610011I04Rik | −1.885885791 | null | unknownP | 2.04E−01 |
| 1418411_at | Fbxl8 | −1.884189604 | GO:0006512 | ubiquitin cycle | 3.08E−03 |
| 1447901_x_at | Sfi1 | −1.797938879 | null | unknownP | 2.04E−01 |
| 1451115_at | Pias3 | −1.778033499 | GO:0006512 | ubiquitin cycle | 3.08E−03 |
| 1416538_at | Ysg2 | −1.728551325 | null | unknownP | 2.04E−01 |
| 1441885_s_at | na | −1.666293232 | null | unknownP | 2.04E−01 |
| 1417896_at | Tjp3 | −1.580284643 | null | unknownP | 2.04E−01 |
| 1439857_at | Usp32 | −1.555573141 | null | unknownP | 2.04E−01 |
| 1449354_at | U2af1-rs1 | −1.548084764 | null | unknownP | 2.04E−01 |
| 1441319_at | Rbm5 | −1.54504959 | GO:0000004 | biological process unknown | 9.65E−02 |
| 1430896_s_at | Nudt7 | −1.534925926 | GO:0015938 | coenzyme A catabolism | 2.08E−05 |
| 1446598_at | Prkca | −1.532847936 | null | unknownP | 2.04E−01 |
| 1447105_at | na | −1.518779997 | null | unknownP | 2.04E−01 |
| 1424906_at | Pqlc3 | −1.511859109 | nuil | unknownP | 2.04E−01 |
| 1442427_at | 9630026M06Rik | −1.507828387 | null | unknownP | 2.04E−01 |
| 1448104_at | Aldh6a1 | −1.506711706 | GO:0008152 | metabolism | 1.28E−02 |
| 1419557_a_at | Tmem9 | −1.497471971 | GO:0006810 | transport | 2.31E−01 |
| 1429351_at | Klhl24 | −1.483148711 | nuil | unknownP | 2.04E−01 |
| 1434193_at | Zmym6 | −1.457894796 | null | unknownP | 2.04E−01 |
| 1434581_at | na | −1.455433574 | null | unknownP | 2.04E−01 |
| 1417218_at | 2810048G17Rik | −1.43831543 | null | unknownP | 2.04E−01 |
| 1440897_at | na | −1.438296402 | null | unknownP | 2.04E−01 |
| 1447112_s_at | Cryl1 | −1.438004747 | GO:0006631 | fatty acid metabolism | 4.77E−03 |
| 1434060_at | Herc1 | −1.434063158 | null | unknownP | 2.04E−01 |
| 1417066_at | Cabc1 | −1.431967873 | GO:0006457 | protein folding | 2.98E−02 |
| 1442315_at | AI426778 | −1.428334819 | null | unknownP | 2.04E−01 |
| 1438155_x_at | Pigo | −1.42673405 | GO:0009117 | nucleotide metabolism | 2.23E−03 |
| 1433593_at | Ypel5 | −1.42626537 | GO:0000004 | biological process unknown | 9.65E−02 |
| 1455602_x_at | C430010P07Rik | −1.42303244 | null | unknownP | 2.04E−01 |
| 1460257_a_at | Mthfs | −1.422588883 | G0:0008152 | metabolism | 1.28E−02 |
| 1447738_s_at | Ankrd13d | −1.421336365 | null | unknownP | 2.04E−01 |
| 1439079_a_at | Erbb2ip | −1.412999179 | GO:0006605 | protein targeting | 1.91E−02 |
| 1429689_at | 4932433N03Rik | −1.412810128 | null | unknownP | 2.04E−01 |
| 1421948_a_at | 2610507L03Rik | −1.397036044 | null | unknownP | 2.04E−01 |
| 1438415_s_at | Yipf2 | −1.390341353 | null | unknownP | 2.04E−01 |
| 1425684_at | 2310005E10Rik | −1.378218217 | null | unknownP | 2.04E−01 |
| 1435345_at | 2600006K01Rik | −1.375904328 | null | unknownP | 2.04E−01 |
| 1434670_at | Kif5a | −1.361096401 | GO:0007018 | microtubule-based movement | 5.89E−03 |
| 1424988_at | Mylip | −1.359416183 | GO:0006512 | ubiquitin cycle | 3.08E−03 |
| 1428447_at | Tmem14a | −1.356743685 | null | unknownP | 2.04E−01 |
| 1424621_at | AA792894 | −1.355659364 | null | unknownP | 2.04E−01 |
| 1448625_at | Golga2 | −1.352622502 | null | unknownP | 2.04E−01 |
| 1444235_at | na | −1.351686534 | null | unknownP | 2.04E−01 |
| 1440533_at | Bfar | −1.347482782 | GO:0006916 | anti-apoptosis | 5.98E−03 |

[symbol]

indicates data missing or illegible when filed

1) A method for analyzing differential gene expression associated with cancer disease, in particular the G1 phase of the cell cycle, such as myeloproliferative disorders (MPD) or breast cancer comprising detection of upregulation and/or downregulation of a pool of polynucleotide sequences in a cell or tissue sample, said pool corresponding to all or part the polynucleotide sequences, subsequences or complements thereof, of the genes listed in Tables 1, 2 and 3.

2) The method according to claim 1 wherein the predefined polynucleotide sequences correspond to all or part of the 188 upregulated genes/EST of Table 2.

3) The method according to claim 1 wherein the predefined polynucleotide sequences correspond to all or part of the 48 downregulated genes/EST of Table 3.

4) The method according to claim 1 wherein the detection of the upregulation of a pool of polynucleotide sequences is performed on a pool of polynucleotide sequences selected from at least one of the genes encoding nucleolar proteins (CIRH1A, LARP1, NOL1, NOL11, NOL5, NOL5A, NOLA1, NOLA2, NOLC1, MKI67IP, SFRS2, SURF6), ribosomal proteins (RPL3, RPL12, RPL41, RPS9, RRS1), small nuclear ribonucleoproteins and interactors (U3/MPHOSPH10, LSM2, RNU22, RNU3IP2), components of RNA polymerase I (POLR1A, POLR1B), II (POLR2H, TAF9) and III (POLR3E, POLR3H), DEAD-box (DDX18, DDX56) and WD repeat (WDR4, WDR43, WDR74, WDR77, GRWD1, PWP1) proteins, eukaryotic initiation and elongation factors (EIF1A, EIF3S1, EIF3S4, EEF1E1), and components of the exosome (EXOSC1, EXOSC2, EXOSC6).

5) The method according to claims 1 or 4, wherein the detection of the upregulation of a pool of polynucleotide sequences is performed on a pool of polynucleotide sequences selected from at least one of the genes encoding proteins of the NOL5A-associated preribosomal ribonucleoprotein complex involved in pre-rRNA processing: NOL5A, PPAN, NOLC1, and BXDC2.

6) The method according to any of claims 1 or 4, wherein the detection of the upregulation of a pool of polynucleotide sequences is performed on a pool of polynucleotide sequences comprising the gene encoding EBNA1BP2.

7) The method according to any of claims 1 or 4, wherein the detection of the upregulation of a pool of polynucleotide sequences is performed on a pool of polynucleotide sequences comprising the gene encoding GAS5, a non-protein-coding multiple small nucleolar RNA (snoRNA).

8) The method according to any of claims 1 or 4, wherein the detection of the upregulation of a pool of polynucleotide sequences is performed on a pool of polynucleotide sequences selected from at least one of the genes encoding protein folding, ubiquitin-dependent protein catabolism, nuclear mRNA splicing via spliceosome and regulation of cell cycle.

9) The method according to any of claims 1 or 4, wherein the detection of the upregulation of a pool of polynucleotide sequences is performed on a pool of polynucleotide sequences selected from the genes encoding CCND2 (cyclin D2) and CDC25A.

10) The method according to any of claims 1 or 4, wherein the detection of the upregulation of a pool of polynucleotide sequences is performed on a pool of polynucleotide sequences comprising at least one of the MYC genes, such as CCND2, CDC25A, DDX18, EBNA1BP2, EEF1E1, MAT2A, MKI67IP, NOL5A, NOLA1, PHB, SFRS2, SHMT1, SLC16A1, SURF6, SRM, RPL3, RPL12, RPL41, RPS9 and RRS1.

11) The method according to claim 1 wherein the detection of the downregulation of a pool of polynucleotide sequences is performed on a pool of polynucleotide sequences comprising the gene encoding PIAS3.

12) A method according to claim 1, wherein said detection is performed on nucleic acids from a tissue sample.

13) A method according to claim 1, wherein said detection is performed on nucleic acids from a tumor cell line.

14) A method according to of claim 1, wherein said detection is performed on DNA microarrays.

15) A polynucleotide library that molecularly characterizes a cancer comprising or corresponding to a pool of polynucleotide sequences either upregulated or down-regulated, said pool corresponding to all or part of the polynucleotide sequences selected from the genes defined in claim 1.

16) A polynucleotide library according to claim 15 immobilized on a solid support.

17) A polynucleotide library according to claim 16 wherein the support is selected from the group comprising at least one of nylon membrane, nitrocellulose membrane, glass slide, glass beads, membranes on glass support or silicon chip, plastic support.

18) A method of prognosis or diagnostic of cancer or for monitoring the treatment of a patient with a cancer comprising the implementation of the method according to claim 1 on nucleic acids from a patient.

19) A method for analysing differential gene expression associated with cancer disease, comprising:
a) obtaining a polynucleotide sample from a patient,
b) reacting said polynucleotide sample obtained in step (a) with a polynucleotide library as defined in claim 15, and
c) detecting the reaction product of step (b).

20) The method according to claim 19 further comprising:
a) obtaining a control polynucleotide sample,
b) reacting said control sample with said polynucleotide library, for example by hybridising the polynucleotide sample with the polynucleotide library,
c) detecting a control sample reaction product, and
d) comparing the amount of said polynucleotide sample reaction product to the amount of said control sample reaction product.

21) A method of prognosis or diagnostic or prediction of tumours susceptible to molecule acting on the G1 phase of the cell cycle, comprising the implementation of the method according to claim 1 on nucleic acids from a patient in order to identify the corresponding signature.

22) The method according to claim 21, wherein the molecule acting on the G1 phase of the cell cycle is rapamycine or its derivatives.

23) A method for monitoring the treatment of a patient with a cancer comprising the implementation of a method according to claim 1 on nucleic acids from a patient.

24) A method of selecting a patient susceptible to be treated with rapamycine and its derivatives or molecule acting on the G1 phase of the cell cycle, comprising the implementation of the method according to claim 1 on nucleic acids from the patient to identify the corresponding signature.

25) The method according to claim 24, wherein the molecule acting on the G1 phase of the cell cycle is rapamycine or its derivatives.
